(19) United States
(12) Patent Application Publication
(10) Pub. No.: US 2007/0225256 A1
(43) Pub. Date: Sep. 27, 2007

(54) COMPOUND

(76) Inventors: Matthew Leese, Slough (GB); Atul Purohit, Slough (GB); Michael John Reed, Slough (GB); Fabrice Jourdan, Slough (GB); Barry Victor Lloyd Potter, Slough (GB); Christian Bubert, Slough (GB)

Correspondence Address: FROMMER LAWRENCE & HAUG, 745 FIFTH AVENUE- 10TH FL., NEW YORK, NY, 10151, US

(21) Appl. No.: 11/689,936

(22) Filed: Mar. 22, 2007

Related U.S. Application Data
(63) Continuation-in-part of application No. PCT/GB05/03641, filed on Sep. 21, 2005.

(30) Foreign Application Priority Data
Sep. 22, 2004 (GB) 0421106.6

(51) Int. Cl. A61K 31/675; A61K 31/58; C07J 43/00; C07J 41/00

(52) U.S. Cl. 514/79; 514/176; 540/5; 540/108

(57) ABSTRACT

The present invention provides a compound comprising a steroidal ring system and an optional group R1 selected from any one of —OH, a sulphamate group, a phosphonate group, a thiophosphonate group, a sulphonate group or a sulphonamide group; wherein the A ring of the steroidal ring system is optionally substituted at position 2 or 4 with a group R⁴ which may be a suitable subtituent, wherein the D ring of the steroidal ring system is substituted by a group R² of the formula -L-R³, wherein L is an optional linker group and R³ is selected from groups which are or which comprise one of (i) —SO₂R⁵, wherein R⁵ is H, a hydrocarbyl group or a bond or group attached to the D ring; (ii) —NO₂; (iii) —SOR⁶, wherein R⁶ is H or a hydrocarbyl group; (iv) —R⁷, wherein R⁷ is a halogen; (v) -alkyl; (vi) —C(═O)R³, wherein R³ is H or hydrocarbyl; (vii) —C≡CR⁹, wherein R⁹ is H or hydrocarbyl; (viii) —OC(═O)NR¹⁰R¹¹ wherein R¹⁰ and R¹¹ are independently selected from H and hydrocarbyl; (ix), (x), (xi), (xii) and (xiii) are formulae wherein when R³ is -alkyl, R⁴ is present as a hydrocarbon group, when R³ is —NO₂R⁴ is present and/or R¹ is present as a sulphamate group, and when R³ is —C(═O)R³R⁴ is present and R¹ is present as a sulphamate group.

### FIELD OF INVENTION

The present invention relates to a compound.

In particular the present invention relates to a compound and to a pharmaceutical composition comprising the compound. The present invention also relates to the use of the compound or composition in therapy applications.

### BACKGROUND TO THE INVENTION

Evidence suggests that oestrogens are the major mitogens involved in promoting the growth of tumours in endocrine-dependent tissues, such as the breast and endometrium. Although plasma oestrogen concentrations are similar in women with or without breast cancer, breast tumour oestrone and oestradiol levels are significantly higher than in normal breast tissue or blood. In situ synthesis of oestrogen is thought to make an important contribution to the high levels of oestrogens in tumours and therefore inhibitors, in particular specific inhibitors, of oestrogen biosynthesis are of potential value for the treatment of endocrine-dependent tumours.

Over the past two decades, there has been considerable interest in the development of inhibitors of the aromatase pathway—which converts the androgen precursor androstenedione to oestrone. However, there is now evidence that the oestrone sulphatase (E1-STS) pathway, i.e. the hydrolysis of oestrone sulphate to oestrone (E1S to E1), and aromatase (i.e. conversion of androstenedione to oestrone) account for the production of oestrogens in breast tumours.

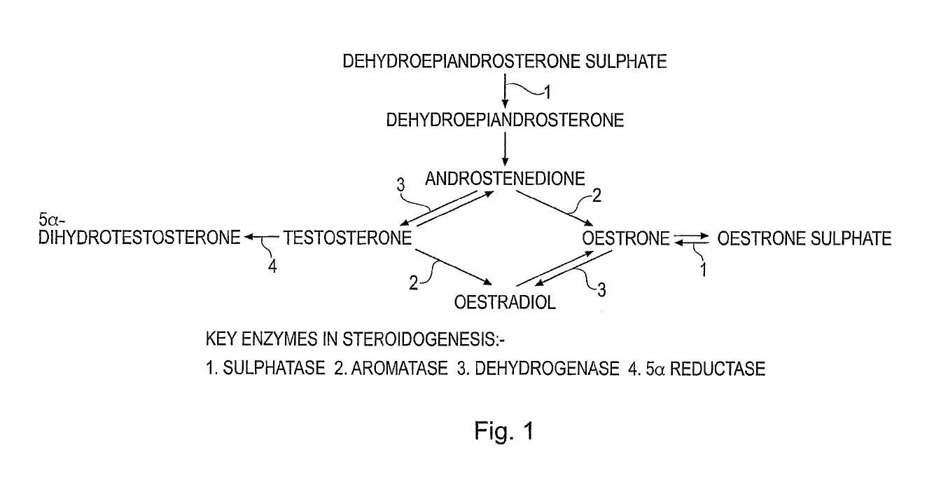

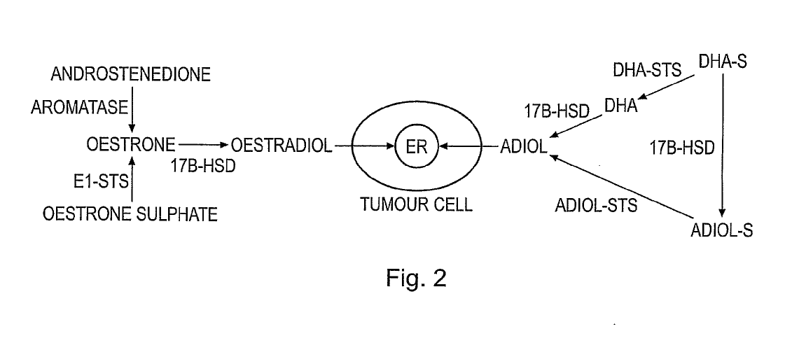

FIGS. 1 and 2 are schematic diagrams showing some of the enzymes involved in the in situ synthesis of oestrone from oestrone sulphate, oestradiol and androstenedione.

In FIG. 2, which schematically shows the origin of oestrogenic steroids in postmenopausal women, “ER” denotes Oestrogen Receptor, “DHA-S” denotes Dehydroepiandrosterone-Sulphate, “Adiol” denotes Androstenediol, “E1-STS” denotes Oestrone Sulphatase, “DHA-STS” denotes DHA-sulphatase, “Adiol-STS” denotes Adiol Sulphatase, and “17B-HSD” denotes Oestradiol 17B-hydroxysteroid dehydrogenase.

As can be seen, the main two enzymes that are involved in the peripheral synthesis of oestrogens are the aromatase enzyme and the enzyme oestrone sulphatase.

In short, the aromatase enzyme converts androstenedione, which is secreted in large amounts by the adrenal cortex, to oestrone. Recent reports have suggested that some flavones could inhibit aromatase activity.

Much of the oestrone so formed, however, is converted to oestrone sulphate (E1S) and there is now a considerable body of evidence showing that E1S in plasma and tissue acts as a reservoir for the formation of oestrone by the action of oestrone sulphatase.

In this regard, it is now believed that the oestrone sulphatase (E1-STS) pathway—i.e. the hydrolysis of oestrone sulphate to oestrone (E1S to E1) is a major source of oestrogen in breast tumours. This theory is supported by a modest reduction of plasma oestrogen concentration in postmenopausal women with breast cancer treated by aromatase inhibitors, such as aminoglutethimide and 4-hydroxyandrostenedione and also by the fact that plasma E1S concentration in these aromatase inhibitor-treated patients remains relatively high. The long half-life of E1S in blood (10-12 h) compared with the unconjugated oestrogens (20 min) and high levels of steroid sulphatase activity in liver and, normal and malignant breast tissues, also lend support to this theory.

Thus, oestrogen formation in malignant breast and endometrial tissues via the sulphatase pathway makes a major contribution to the high concentration of oestrogens which are present in these tumours.

PCT/GB92/01587 teaches novel steroid sulphatase inhibitors and pharmaceutical compositions containing them for use in the treatment of oestrone dependent tumours, especially breast cancer. These steroid sulphatase inhibitors are sulphamate esters, such as N,N-dimethyl oestrone-3-sulphamate and, preferably, oestrone-3-sulphamate (otherwise known as “EMATE”). EMATE has the following structure:

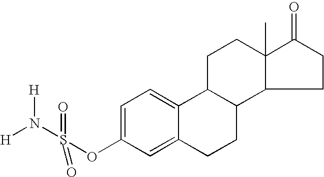

It is known that EMATE is a potent E1-STS inhibitor as it displays more than 99% inhibition of E1-STS activity in intact MCF-7 cells at 0.1 nM. EMATE also inhibits the E1-STS enzyme in a time- and concentration-dependent manner, indicating that it acts as an active site-directed inactivator. Although EMATE was originally designed for the inhibition of E1-STS, it also inhibits dehydroepiandrosterone sulphatase (DHA-STS), which is an enzyme that is believed to have a pivotal role in regulating the biosynthesis of the oestrogenic steroid androstenediol. Also, there is now evidence to suggest that androstenediol may be of even greater importance as a promoter of breast tumour growth. EMATE is also active in vivo as almost complete inhibition of rat liver E1-STS (99%) and DHA-STS (99%) activities resulted when it is administered either orally or subcutaneously. In addition, EMATE has been shown to have a memory enhancing effect in rats. Studies in mice have suggested an association between DHA-STS activity and the regulation of part of the immune response. It is thought that this may also occur in humans. The bridging O-atom of the sulphamate moiety in EMATE is important for inhibitory activity. Thus, when the 3-O-atom is replaced by other heteroatoms as in oestrone-3-N-sulphamate and oestrone-3-S-sulphamate, these analogues are weaker non-time-dependent inactivators.

In addition to oestrone, the other major steroid with oestrogenic properties which is produced by postmenopausal women is androstenediol (see FIG. 2).

Androstenediol, although an androgen, can bind to the oestrogen receptor (ER) and can stimulate the growth of ER positive breast cancer cells and the growth of carcinogen-induced mammary tumours in the rat. Importantly, in postmenopausal women 90% of the androstenediol produced originates from the androgen dehydroepiandrosterone sulphate (DHA-S) which is secreted in large amounts by the adrenal cortex. DHA-S is converted to DHA by DHA sulphatase, which may be the same as, or different from, the enzyme, oestrone sulphatase, which is responsible for the hydrolysis of E1S.

During the last 10-15 years considerable research has also been carried out to develop potent aromatase inhibitors, some of which are now marketed. However, in three recent reports of postmenopausal women with breast cancer who received aromatase inhibitor therapy, plasma E1S concentrations remained between 400-1000 pg/ml.

In summation therefore in situ synthesis of oestrogen is thought to make an important contribution to the high levels of oestrogens in tumours and therefore specific inhibitors of oestrogen biosynthesis are of potential value for the treatment of endocrine-dependent tumours.

Moreover, even though oestrogen formation in malignant breast and endometrial tissues via the sulphatase pathway makes a major contribution to the high concentration of oestrogens, there are still other enzymatic pathways that contribute to in vivo synthesis of oestrogen.

### SUMMARY ASPECTS OF THE PRESENT INVENTION

The present invention is based on the surprising finding that steroidal compounds carrying a specific group on the D ring could be used as effective steroid sulphatase (STS) inhibitors; cell cycling modulators; apoptosis modulators; cell growth modulators; glucose uptake prevention and/or suppression agents; tumour angiogenesis prevention agents or inhibitors; microtubules disruptors; and/or apoptosis inducers.

The compounds of the present invention may comprise other substituents. These other substituents may, for example, further increase the activity of the compounds of the present invention and/or increase stability (ex vivo and/or in vivo).

### DETAILED ASPECTS OF THE PRESENT INVENTION

According to one aspect of the present invention, there is provided a compound comprising a steroidal ring system and an optional group R¹ selected from any one of —OH, a sulphamate group, a phosphonate group, a thiophosphonate group, a sulphonate group or a sulphonamide group; wherein the A ring of the steroidal ring system is optionally substituted at position 2 or 4 with a group R⁴ which may be a suitable subtituent wherein the D ring of the steroidal ring system is substituted by a group R² of the formula -L-R³, wherein L is an optional linker group and R³ is selected from groups which are or which comprise one of (i) —SO₂R⁵, wherein R⁵ is H, a hydrocarbyl group or a bond or group attached to the D ring (ii) —NO₂ (iii) —SOR⁶, wherein R⁶ is H or a hydrocarbyl group (iv) —R⁷, wherein R⁷ is a halogen (v) -alkyl (vi) —C(═O)R⁶, wherein R⁸ is H or hydrocarbyl (vii) —C≡CR⁹, wherein R⁹ is H or hydrocarbyl (viii) —OC(═O)NR¹⁰R¹¹, wherein R¹⁰ and R¹¹ are independently selected from H and hydrocarbyl 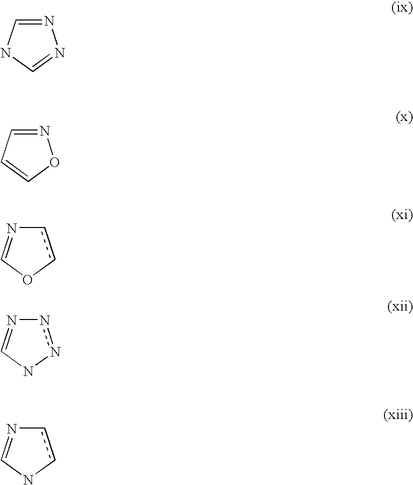
wherein when R³ is -alkyl, R⁴ is present as a hydrocarbon group, when R³ is —NO₂R⁴ is present and/or R¹ is present as a sulphamate group, and when R³ is —C(═O)R⁸R⁴ is present and R¹ is present as a sulphamate group.

According to one aspect of the present invention, there is provided a pharmaceutical composition comprising (a) a compound as defined herein and (b) a pharmaceutically acceptable carrier, diluent, excipient or adjuvant.

According to one aspect of the present invention, there is provided a (i) compound as defined herein, or (ii) composition as defined herein, for use in medicine.

According to one aspect of the present invention, there is provided use of (i) a compound as defined herein, or (ii) a composition as defined herein, in the manufacture of a medicament to prevent and/or inhibit tumour growth.

According to one aspect of the present invention, there is provided use of (i) a compound as defined herein, or (ii) a composition as defined herein, in the manufacture of a medicament for use in the therapy of a condition or disease associated with one or more of steroid sulphatase (STS) activity; cell cycling; apoptosis; cell growth; glucose uptake by a tumour; tumour angiogenesis; microtubules formation; and apoptosis.

According to one aspect of the present invention, there is provided use of (i) a compound as defined herein, or (ii) a composition as defined herein, in the manufacture of a medicament for use in the therapy of a condition or disease associated with one or more of adverse steroid sulphatase (STS) activity; cell cycling; apoptosis; cell growth; glucose uptake by a tumour; tumour angiogenesis; microtubules formation; and apoptosis.

According to one aspect of the present invention, there is provided use of (i) a compound as defined herein, or (ii) a composition as defined herein, in the manufacture of a medicament for one or more of inhibiting steroid sulphatase (STS) activity; modulating cell cycling; modulating apoptosis; modulating cell growth; preventing and/or suppressing glucose uptake by a tumour; preventing and/or inhibiting tumour angiogenesis; disrupting microtubules; and inducing apoptosis.

According to one aspect of the present invention, there is provided use of (i) a compound as defined herein, or (ii) a composition as defined herein, in the manufacture of a medicament for inhibiting steroid sulphatase (STS) activity.

According to one aspect of the present invention, there is provided use of (i) a compound as defined herein, or (ii) a composition as defined herein, in the manufacture of a medicament for modulating cell growth.

According to one aspect of the present invention, there is provided a method of treatment comprising administering to a subject in need of treatment (i) a compound as defined herein, or (ii) a composition as defined herein.

According to one aspect of the present invention, there is provided a method of treatment comprising administering to a subject in need of treatment (i) a compound as defined herein, or (ii) a composition as defined herein, in order to inhibit steroid sulphatase (STS) activity; modulate cell cycling; modulate apoptosis; modulate cell growth; prevent and/or suppress glucose uptake by a tumour; prevent and/or inhibit tumour angiogenesis; disrupt microtubules; and/or induce apoptosis.

According to one aspect of the present invention, there is provided a method comprising (a) performing an assay for one or more of steroid sulphatase (STS) inhibition; cell cycling modulation; apoptosis modulation; cell growth modulation; prevention and/or suppression of glucose uptake by a tumour; tumour angiogenesis prevention and/or inhibition; microtubules disruption; and apoptosis induction, with one or more candidate compounds defined herein; (b) determining whether one or more of said candidate compounds is/are capable of one or more of steroid sulphatase (STS) inhibition; cell cycling modulation; apoptosis modulation; cell growth modulation; prevention and/or suppression of glucose uptake by a tumour; tumour angiogenesis prevention and/or inhibition; microtubules disruption; and apoptosis induction; and (c) selecting one or more of said candidate compounds that is/are capable of one or more of steroid sulphatase (STS) inhibition; cell cycling modulation; apoptosis modulation; cell growth modulation; prevention and/or suppression of glucose uptake by a tumour; tumour angiogenesis prevention and/or inhibition; microtubules disruption; and apoptosis induction.

In any one of the methods of the present invention, one or more additional steps may be present. For example, the method may also include the step of modifying the identified candidate compound (such as by chemical and/or enzymatic techniques) and the optional additional step of testing that modified compound for one or more of steroid sulphatase (STS) inhibition; cell cycling modulation; apoptosis modulation; cell growth modulation; prevention and/or suppression of glucose uptake by a tumour; tumour angiogenesis prevention and/or inhibition; microtubules disruption; and apoptosis induction. By way of further example, the method may also include the step of determining the structure (such as by use of crystallographic techniques) of the identified candidate compound and then performing computer modelling studies—such as to further increase its action. Thus, the present invention also encompasses a computer having a dataset (such as the crystallographic co-ordinates) for said identified candidate compound. The present invention also encompasses that identified candidate compound when presented on a computer screen for the analysis thereof—such as enzyme and/or protein binding studies.

According to one aspect of the present invention, there is provided a compound identified by the method of the present invention.

The present invention also encompasses the novel compounds of the present invention (such as those presented herein), as well as processes for making same (such as the processes presented herein) as well as novel intermediates (such as those presented herein) for use in those processes.

Broad Aspects

According to one broad aspect of the present invention, there is provided use of a compound in the manufacture of a medicament for use in the therapy of a condition or disease associated with one or more of cell cycling; apoptosis; cell growth; glucose uptake by a tumour; tumour angiogenesis; microtubules formation; and apoptosis; wherein the compound comprises a steroidal ring system and an optional group R¹ selected from any one of —OH, a sulphamate group, a phosphonate group, a thiophosphonate group, a sulphonate group or a sulphonamide group; wherein the A ring of the steroidal ring system is optionally substituted at position 2 or 4 with a group R⁴ which may be a suitable subtituent wherein the D ring of the steroidal ring system is substituted by a group R² of the formula -L-R³, wherein L is an optional linker group and R³ is selected from groups which are or which comprise one of (i) —SO₂R⁵, wherein R⁵ is H, a hydrocarbyl group or a bond or group attached to the D ring (ii) —NO₂ (iii) —SOR⁶, wherein R⁵ is H or a hydrocarbyl group (iv) —R⁷, wherein R⁷ is a halogen (v) -alkyl (vi) —C(═O)R⁸, wherein R⁸ is H or hydrocarbyl (vii) —C≡CR⁹, wherein R⁹ is H or hydrocarbyl (viii) —OC(═O)NR¹⁰R¹¹, wherein R¹⁰ and R¹¹ are independently selected from H and hydrocarbyl

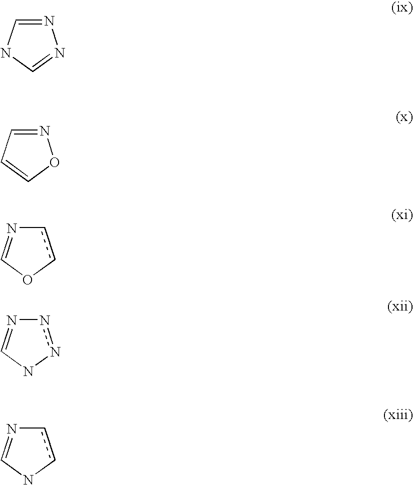

According to another broad aspect of the present invention, there is provided use of a compound in the manufacture of a medicament for use in the therapy of a condition or disease associated with one or more of adverse cell cycling; apoptosis; cell growth; glucose uptake by a tumour; tumour angiogenesis; microtubules formation; and apoptosis; wherein the compound comprises a steroidal ring system and an optional group R¹ selected from any one of —OH, a sulphamate group, a phosphonate group, a thiophosphonate group, a sulphonate group or a sulphonamide group; wherein the A ring of the steroidal ring system is optionally substituted at position 2 or 4 with a group R⁴ which may be a suitable subtituent wherein the D ring of the steroidal ring system is substituted by a group R² of the formula -L-R³, wherein L is an optional linker group and R³ is selected from groups which are or which comprise one of (i) —SO₂R⁵, wherein R⁵ is H, a hydrocarbyl group or a bond or group attached to the D ring (ii) —NO₂ (iii) —SOR⁶, wherein R⁶ is H or a hydrocarbyl group (iv) —R⁷, wherein R⁷ is a halogen (v) -alkyl (vi) —C(═O)R⁸, wherein R⁸ is H or hydrocarbyl (vii) —C≡CR⁹, wherein R⁹ is H or hydrocarbyl (viii) —OC(═O)NR¹⁰R¹¹, wherein R¹⁰ and R¹¹ are independently selected from H and hydrocarbyl

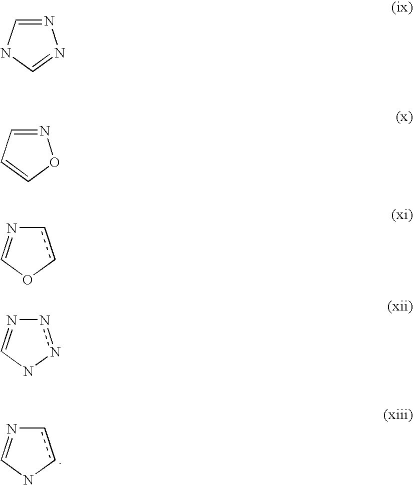

According to a further broad aspect of the present invention, there is provided use of a compound in the manufacture of a medicament for one or more of modulating cell cycling; modulating apoptosis; modulating cell growth; preventing and/or suppressing glucose uptake by a tumour; preventing and/or inhibiting tumour angiogenesis; disrupting microtubules; and inducing apoptosis; wherein the compound comprises a compound comprising a steroidal ring system and an optional group R¹ selected from any one of —OH, a sulphamate group, a phosphonate group, a thiophosphonate group, a sulphonate group or a sulphonamide group; wherein the A ring of the steroidal ring system is optionally substituted at position 2 or 4 with a group R⁴ which may be a suitable subtituent wherein the D ring of the steroidal ring system is substituted by a group R² of the formula -L-R³, wherein L is an optional linker group and R³ is selected from groups which are or which comprise one of (i) —SO₂R⁵, wherein R⁵ is H, a hydrocarbyl group or a bond or group attached to the D ring (ii) —NO₂ (iii) —SOR⁶, wherein R⁶ is H or a hydrocarbyl group (iv) —R⁷, wherein R⁷ is a halogen (v) -alkyl (vi) —C(═O)R⁸, wherein R⁸ is H or hydrocarbyl (vii) —C≡CR⁹, wherein R⁹ is H or hydrocarbyl (viii) —OC(═O)NR¹⁰R¹¹, wherein R¹⁰ and R¹¹ are independently selected from H and hydrocarbyl

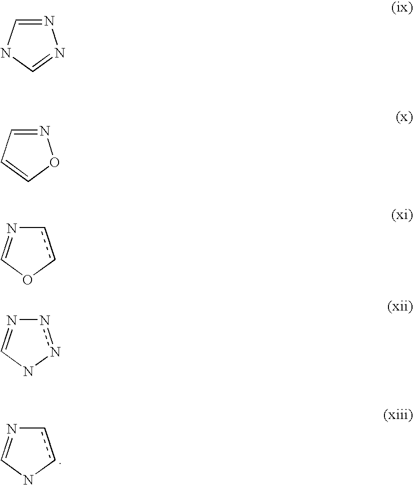

According to a broad aspect of the present invention, there is provided use of a compound in the manufacture of a medicament for modulating cell growth; wherein the compound comprises a steroidal ring system and an optional group R¹ selected from any one of —OH, a sulphamate group, a phosphonate group, a thiophosphonate group, a sulphonate group or a sulphonamide group; wherein the A ring of the steroidal ring system is optionally substituted at position 2 or 4 with a group R⁴ which may be a suitable subtituent wherein the D ring of the steroidal ring system is substituted by a group R² of the formula -L-R³, wherein L is an optional linker group and R³ is selected from groups which are or which comprise one of (i) —SO₂R⁵, wherein R⁵ is H, a hydrocarbyl group or a bond or group attached to the D ring (ii) —NO₂ (iii) —SOR⁶, wherein R⁶ is H or a hydrocarbyl group (iv) —R⁷, wherein R⁷ is a halogen (v) -alkyl (vi) —C(═O)R⁸, wherein R⁸ is H or hydrocarbyl (vii) —C≡CR⁹, wherein R⁹ is H or hydrocarbyl (viii) —OC(═O)NR¹⁰R¹¹, wherein R¹⁰ and R¹¹ are independently selected from H and hydrocarbyl

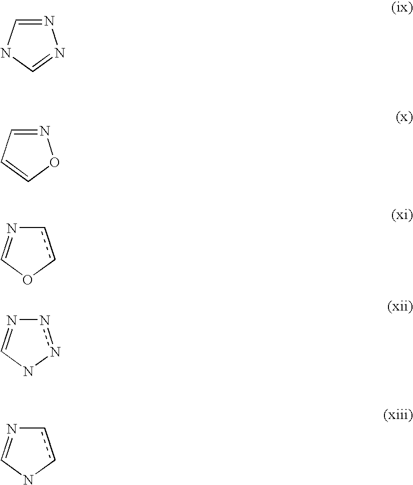

According to a broad aspect of the present invention, there is provided a method of treatment comprising administering to a subject in need of treatment a compound in order to modulate cell cycling; modulate apoptosis; modulate cell growth; prevent and/or suppress glucose uptake by a tumour; prevent and/or inhibit tumour angiogenesis; disrupt microtubules; and/or induce apoptosis wherein the compound comprises a steroidal ring system and an optional group R¹ selected from any one of —OH, a sulphamate group, a phosphonate group, a thiophosphonate group, a sulphonate group or a sulphonamide group; wherein the A ring of the steroidal ring system is optionally substituted at position 2 or 4 with a group R⁴ which may be a suitable subtituent wherein the D ring of the steroidal ring system is substituted by a group R² of the formula -L-R³, wherein L is an optional linker group and R³ is selected from groups which are or which comprise one of (i) —SO₂R⁵, wherein R⁵ is H, a hydrocarbyl group or a bond or group attached to the D ring (ii) —NO₂ (iii) —SOR⁶, wherein R⁶ is H or a hydrocarbyl group (iv) —R⁷, wherein R⁷ is a halogen (v) -alkyl (vi) —C(═O)R⁸, wherein R⁸ is H or hydrocarbyl (vii) —C≡CR⁹, wherein R⁹ is H or hydrocarbyl (viii) —OC(═O)NR¹⁰R¹¹, wherein R¹⁰ and R¹¹ are independently selected from H and hydrocarbyl

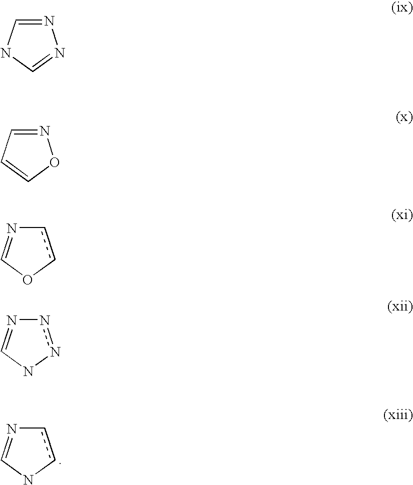

According to one broad aspect of the present invention, there is provided use of a compound in the manufacture of a medicament for use in the therapy of a condition or disease associated with carbonic anhydrase; wherein the compound comprises a steroidal ring system and an optional group R¹ selected from any one of —OH, a sulphamate group, a phosphonate group, a thiophosphonate group, a sulphonate group or a sulphonamide group; wherein the A ring of the steroidal ring system is optionally substituted at position 2 or 4 with a group R⁴ which may be a suitable subtituent wherein the D ring of the steroidal ring system is substituted by a group R² of the formula -L-R³, wherein L is an optional linker group and R³ is selected from groups which are or which comprise one of (i) —SO₂R⁵, wherein R⁵ is H, a hydrocarbyl group or a bond or group attached to the D ring (ii) —NO₂ (iii) —SOR⁶, wherein R⁶ is H or a hydrocarbyl group (iv) —R⁷, wherein R⁷ is a halogen (v) -alkyl (vi) —C(═O)R⁸, wherein R⁸ is H or hydrocarbyl (vii) —C≡CR⁹, wherein R⁹ is H or hydrocarbyl (viii) —OC(═O)NR¹⁰R¹¹, wherein R¹⁰ and R¹¹ are independently selected from H and hydrocarbyl

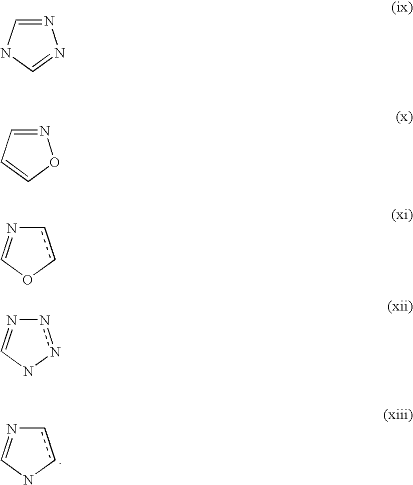

According to another broad aspect of the present invention, there is provided use of a compound in the manufacture of a medicament for use in the therapy of a condition or disease associated with adverse carbonic anhydrase activity; wherein the compound comprises a steroidal ring system and an optional group R¹ selected from any one of —OH, a sulphamate group, a phosphonate group, a thiophosphonate group, a sulphonate group or a sulphonamide group; wherein the A ring of the steroidal ring system is optionally substituted at position 2 or 4 with a group R⁴ which may be a suitable subtituent wherein the D ring of the steroidal ring system is substituted by a group R² of the formula -L-R³, wherein L is an optional linker group and R³ is selected from groups which are or which comprise one of (i) —SO₂R⁵, wherein R⁵ is H, a hydrocarbyl group or a bond or group attached to the D ring (ii) —NO₂ (iii) —SOR⁶, wherein R⁵ is H or a hydrocarbyl group (iv) —R⁷, wherein R⁷ is a halogen (v) -alkyl (vi) —C(═O)R⁸, wherein R⁸ is H or hydrocarbyl (vii) —C≡CR⁹, wherein R⁹ is H or hydrocarbyl (viii) —OC(═O)NR¹⁰R¹¹, wherein R¹⁰ and R¹¹ are independently selected from H and hydrocarbyl

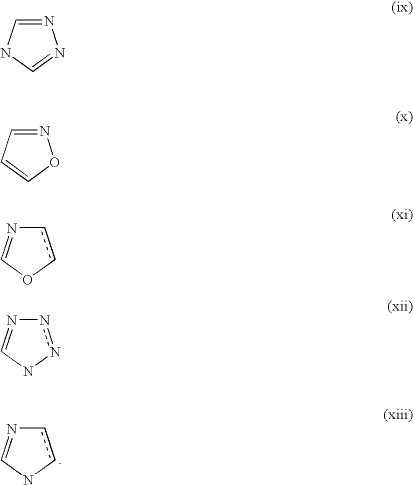

According to a further broad aspect of the present invention, there is provided use of a compound in the manufacture of a medicament for modulating carbonic anhydrase activity; wherein the compound comprises a compound comprising a steroidal ring system and an optional group R¹ selected from any one of —OH, a sulphamate group, a phosphonate group, a thiophosphonate group, a sulphonate group or a sulphonamide group; wherein the A ring of the steroidal ring system is optionally substituted at position 2 or 4 with a group R⁴ which may be a suitable subtituent wherein the D ring of the steroidal ring system is substituted by a group R² of the formula -L-R³, wherein L is an optional linker group and R³ is selected from groups which are or which comprise one of (i) —SO₂R⁵, wherein R⁵ is H, a hydrocarbyl group or a bond or group attached to the D ring (ii) —NO₂ (iii) —SOR⁶, wherein R⁶ is H or a hydrocarbyl group (iv) —R⁷, wherein R⁷ is a halogen (v) -alkyl (vi) —C(═O)R⁸, wherein R⁸ is H or hydrocarbyl (vii) —C≡CR⁹, wherein R⁹ is H or hydrocarbyl (viii) —OC(═O)NR¹⁰R¹¹, wherein R¹⁰ and R¹¹ are independently selected from H and hydrocarbyl

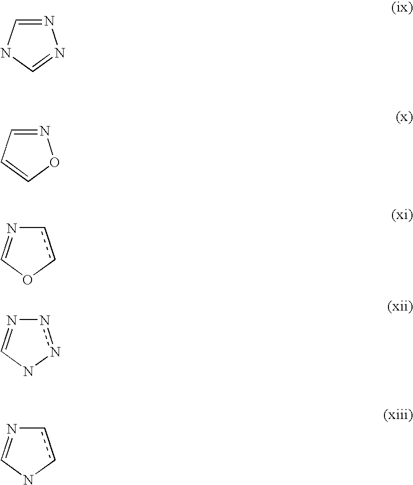

According to a broad aspect of the present invention, there is provided a method of treatment comprising administering to a subject in need of treatment a compound in order to modulate carbonic anhydrase activity; wherein the compound comprises a steroidal ring system and an optional group R¹ selected from any one of —OH, a sulphamate group, a phosphonate group, a thiophosphonate group, a sulphonate group or a sulphonamide group; wherein the A ring of the steroidal ring system is optionally substituted at position 2 or 4 with a group R⁴ which may be a suitable subtituent wherein the D ring of the steroidal ring system is substituted by a group R² of the formula -L-R³, wherein L is an optional linker group and R³ is selected from groups which are or which comprise one of (i) —SO₂R⁵, wherein R⁹ is H, a hydrocarbyl group or a bond or group attached to the D ring (ii) —NO₂ (iii) —SOR⁶, wherein R⁶ is H or a hydrocarbyl group (iv) —R⁷, wherein R⁷ is a halogen (v) -alkyl (vi) —C(═O)R⁸, wherein R⁸ is H or hydrocarbyl (vii) —C≡CR⁹, wherein R⁹ is H or hydrocarbyl (viii) —OC(═O)NR¹⁰R¹¹, wherein R¹⁰ and R¹¹ are independently selected from H and hydrocarbyl

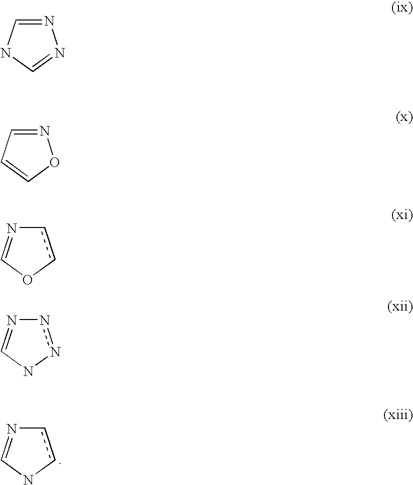

In these broad aspects, preferably R¹ to R¹¹ and L are as herein defined.

For ease of reference, these and further aspects of the present invention are now discussed under appropriate section headings. However, the teachings under each section are not necessarily limited to each particular section.

Some Advantages

One key advantage of the present invention is that the compounds of the present invention can prevent and/or inhibit tumour angiogenesis.

One key advantage of the present invention is that the compounds of the present invention can modulate cell cycling.

One key advantage of the present invention is that the compounds of the present invention can modulate apoptosis.

One key advantage of the present invention is that the compounds of the present invention can modulate cell growth.

One key advantage of the present invention is that the compounds of the present invention can prevent and/or suppress glucose uptake by a tumour.

One key advantage of the present invention is that the compounds of the present invention can inhibit steroid sulphatase (STS) activity.

One key advantage of the present invention is that the compounds of the present invention can disrupt microtubules.

In this respect, microtubules, together with microfilaments and intermediate filaments form part of the cytoskeletal system of a cell. Microtubules are responsible for many of cell movements—examples include the beating of cilia and flagella and the transport of membrane vesicles in the cytoplasm. All these movements result from the polymerisation and depolymerisation of microtubules or the actions of the microtubule motor proteins dynein and kinesins. Some other cell movements, such as the alignment and separation of chromosomes during meiosis and mitosis result from both mechanisms. Microtubules also direct cell movement but in some cases, microtubules serve purely structural functions.

A microtubule is composed of subunits that are heterodimers of α-tubulin and β-tubulin monomers. There are two populations of microtubules: stable, long-lived microtubules and dynamic, short lived microtubules. Dynamic microtubules are found when the microtubule structures need to assemble and dissemble quickly. For example, during mitosis, the cytosolic microtubule network characteristic of interphase cells disappears and the tubulin from it is used to form the spindle apparatus which partitions chromosomes equally to the daughter cells. When mitosis is complete, the spindle disassembles and the interphase microtubule network reforms.

Drugs that inhibit mitosis provide a useful means to manipulate the microtubules in a cell. Three drugs: colchicine, vinblastine and taxol—all purified from plants—have proved to be very powerful probes of microtubule function partly because they bind only to tubulin or microtubules and not to other proteins and also because their concentrations in cells can be easily controlled.

Because of their effects on mitosis, microtubule inhibitors have been widely used to treat illness and more recently as anticancer agents, since blockage of spindle formation will preferentially inhibit rapidly dividing cells like cancer cells. A highly effective anti-ovarian cancer agent is taxol. In ovarian cancer cells, which undergo rapid cell divisions, mitosis is blocked by taxol treatment while other functions carried out by intact microtubules are not affected. A comprehensive review of microtubules can be found in “Molecular Cell Biology” (Ed: Lodish et al 1995 WH Freeman and Co. New York pp 1051-1122).

One key advantage of the present invention is that the compounds of the present invention can induce apoptosis.

Apoptosis is induced by MT-targeting drugs, a process which may involve the phosphorylation (and inactivation) of the apoptosis regulator, the bcl-2 protein (Halder, Cancer Res. 57: 229, 1997).

The present invention is based on the surprising finding that the compound provides an effective treatment of cancer.

Another advantage of the compounds of the present invention is that they may be potent in vivo.

Some of the compounds of the present invention may be non-oestrogenic compounds. Here, the term “non-oestrogenic” means exhibiting no or substantially no oestrogenic activity. Here, by the term “non-oestrogenic” means exhibiting no or substantially no systemic oestrogenic activity, such as that determined by Protocol 4.

For some applications, the compounds have an oestrogenic effect.

Another advantage is that some of the compounds may not be capable of being metabolised to compounds which display or induce hormonal activity.

For some applications, preferably the compounds have a reversible action.

For some applications, preferably the compounds have an irreversible action.

Some of the compounds of the present invention are also advantageous in that they may be orally active.

Some of the compounds of the present invention may useful for the prevention and/or treatment of cancer, such as breast cancer, as well as (or in the alternative) non-malignant conditions, such as the prevention and/or treatment of inflammatory conditions—such as conditions associated with any one or more of: autoimmunity, including for example, rheumatoid arthritis, type I and II diabetes, systemic lupus erythematosus, multiple sclerosis, myasthenia gravis, thyroiditis, vasculitis, ulcerative colitis and Crohn's disease, skin disorders e.g. acne, psoriasis and contact dermatitis; graft versus host disease; eczema; asthma and organ rejection following transplantation. The compounds of the present invention are useful particularly when pharmaceuticals may need to be administered from an early age.

In one embodiment, the compounds of the present invention are useful for the treatment of breast cancer.

Thus, some of the compounds of the present invention are also believed to have therapeutic uses other than for the treatment of endocrine-dependent cancers, such as the treatment of autoimmune diseases.

For ease of reference, these and further aspects of the present invention are now discussed under appropriate section headings. However, the teachings under each section are not necessarily limited to each particular section.

Preferable Aspects

Compound

As described above the present invention provides a compound comprising a steroidal ring system and an optional group R¹ selected from any one of —OH, a sulphamate group, a phosphonate group, a thiophosphonate group, a sulphonate group or a sulphonamide group; wherein the D ring of the steroidal ring system is substituted by a group R² of the formula -L-R³, wherein L is an optional linker group and R³ is selected from groups which are or which comprise one of (i) —SO₂R⁵, wherein R⁵ is H, a hydrocarbyl group or a bond or group attached to the D ring (ii) —NO₂ (iii) —SOR⁶, wherein R⁶ is H or a hydrocarbyl group (iv) —R⁷, wherein R⁷ is a halogen (v) -alkyl (vi) —C(═O)R⁸, wherein R⁸ is H or hydrocarbyl (vii) —C≡CR⁹, wherein R⁹ is H or hydrocarbyl (viii) —OC(═O)NR¹⁰R¹¹, wherein R¹⁰ and R¹¹ are independently selected from H and hydrocarbyl

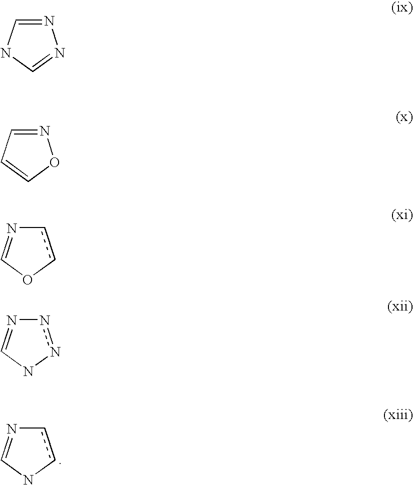

In one preferred aspect the compound is capable of one or more of inhibiting steroid sulphatase (STS) activity; modulating cell cycling; modulating apoptosis; modulating cell growth; preventing and/or suppressing glucose uptake by a tumour; preventing and/or inhibiting tumour angiogenesis; disrupting microtubules; and inducing apoptosis.

Steroidal Ring System

The compound of the present invention has a steroidal ring component—that is to say a cyclopentanophenanthrene skeleton, or bio-isosteres thereof.

As is well known in the art, a classical steroidal ring structure has the generic formula of:

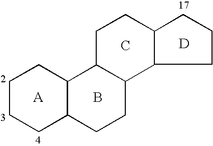

In the above formula, the rings have been labelled and numbered in the conventional manner.

In one aspect, the steroidal ring structure may contain any one or more of C, H, O, N, P, halogen (including Cl, Br and I), S and P.

At least one of the cyclic groups of the steroidal ring structure may be a heterocyclic group (a heterocycle) or a non-heterocyclic group.

At least one of the cyclic groups of the steroidal ring structure may be a saturated ring structure or an unsaturated ring structure (such as an aryl group).

Preferably, at least one of the cyclic groups of the steroidal ring structure is an aryl ring.

An example of a bio-isostere is when any one or more of rings A, B, C and D is a heterocyclic ring and/or when any one or more of rings A, B, C and D has been substituted and/or when any one or more of rings A, B, C and D has been modified; but wherein the bio-isostere has steroidal properties.

In this regard, the structure of a preferred steroidal ring structure can be presented as: 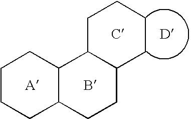
wherein each ring A′, B′, C′ and D′ independently represents a heterocyclic ring or a non-heterocyclic ring, which rings may be independently substituted or unsubstituted, saturated or unsaturated.

By way of example, any one or more of rings A′, B′, C′ and D′ may be independently substituted with suitable groups—such as an alkyl group, an allyl group, an hydroxy group, a halo group, a hydrocarbyl group, an oxyhydrocarbyl group etc.

The term “hydrocarbyl group” as used herein means a group comprising at least C and H and may optionally comprise one or more other suitable substituents. Examples of such substituents may include halo-, alkoxy-, nitro-, a hydrocarbon group, an N-acyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen.

In one preferred embodiment of the present invention, the hydrocarbyl group is a hydrocarbon group.

Here the term “hydrocarbon” means any one of an alkyl group, an alkenyl group, an alkynyl group, an acyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

In one preferred embodiment of the present invention, the hydrocarbyl group is an oxyhydrocarbyl group.

The term “oxyhydrocarbyl group” as used herein means a group comprising at least C, H and O and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the oxyhydrocarbyl group comprises more than one C then those carbons need not necessarily be linked to each other. For example, at least two of the carbons may be linked via a suitable element or group. Thus, the oxyhydrocarbyl group may contain hetero atoms. Suitable hetero atoms will be apparent to those skilled in the art and include, for instance, sulphur and nitrogen.

In one preferred embodiment of the present invention, the oxyhydrocarbyl group is a oxyhydrocarbon group.

Here the term “oxyhydrocarbon” means any one of an alkoxy group, an oxyalkenyl group, an oxyalkynyl group, which groups may be linear, branched or cyclic, or an oxyaryl group. The term oxyhydrocarbon also includes those groups but wherein they have been optionally substituted. If the oxyhydrocarbon is a branched structure having substituent(s) thereon, then the substitution may be on either the hydrocarbon backbone or on the branch; alternatively the substitutions may be on the hydrocarbon backbone and on the branch.

Preferably the oxyhydrocarbyl group is an alkoxy group. Preferably the oxyhydrocarbyl group is of the formula C₁₋₆O (such as a C₁₋₃O).

An example of D′ is a five or six membered non-heterocyclic ring having at least one substituent.

In one preferred embodiment, the ring D′ is substituted with a ethinyl group.

If any one of rings A′, B′, C′ and D′ is a heterocyclic ring, then preferably that heterocyclic ring comprises a combination of C atoms and at least one N atom and/or at least one O atom. Other heterocyclic atoms may be present in the ring.

Examples of suitable, preferred steroidal nuclei rings A′-D′ of the compounds of the present invention include rings A-D of oestrone and dehydroepiandrosterone.

Preferred steroidal nuclei rings A′-D′ of the compounds of the present invention include rings A-D of:

oestrones and substituted oestrones, viz:

oestrone

2-OH-oestrone

2-alkoxy-oestrone (such as C₁₋₆ alkoxy-oestrone, such as 2-methoxy-oestrone)

4-OH-oestrone

6α-OH-oestrone

7α-OH-oestrone

16α-OH-oestrone

16β-OH-oestrone

oestradiols and substituted oestradiols, viz:

2-OH-17β-oestradiol

2-alkoxy-17β-oestradiol (such as C₁₋₆ alkoxy-17β-oestradiol, such as 2-methoxy-17β-oestradiol)

4-OH-17β-oestradiol

6α-OH-17β-oestradiol

7α-OH-17β-oestradiol

2-OH-17α-oestradiol

2-alkoxy-17α-oestradiol (such as C₁₋₆ alkoxy-17α-oestradiol, such as 2-methoxy-17α-oestradiol)

4-OH-17α-oestradiol

6α-OH-17α-oestradiol

7α-OH-17α-oestradiol

16α-OH-17α-oestradiol

16α-OH-17β-oestradiol

16β-OH-17α-oestradiol

16β-OH-17β-oestradiol

17α-oestradiol

17β-oestradiol

17α-ethinyl-17β-oestradiol

17β-ethinyl-17α-oestradiol

oestriols and substituted oestriols, viz:

oestriol

2-OH-oestriol

2-alkoxy-oestriol (such as C₁₋₆ alkoxy-oestriol, such as 2-methoxy-oestriol)

4-OH-oestriol

6α-OH-oestriol

7α-OH-oestriol

dehydroepiandrosterones and substituted dehydroepiandrosterones, viz:

dehydroepiandrosterones

6α-OH-dehydroepiandrosterone

7α-OH-dehydroepiandrosterone

16α-OH-dehydroepiandrosterone

16β-OH-dehydroepiandrosterone

In general terms the ring system A′B′C′D′ may contain a variety of non-interfering substituents. In particular, the ring system A′B′C′D′ may contain one or more hydroxy, alkyl especially lower (C₁-C₆) alkyl, e.g. methyl, ethyl, n-propyl, isopropyl, n-butyl, sec-butyl, tert-butyl, n-pentyl and other pentyl isomers, and n-hexyl and other hexyl isomers, alkoxy especially lower (C₁-C₆) alkoxy, e.g. methoxy, ethoxy, propoxy etc., alkinyl, e.g. ethinyl, or halogen, e.g. fluoro substituents.

In an alternative embodiment, the polycyclic compound may not contain or be based on a steroid nucleus. In this regard, the polycyclic compound may contain or be based on a non-steroidal ring system—such as diethylstilboestrol, stilboestrol, coumarins, and other ring systems. Other suitable non-steroidal compounds for use in or as the composition of the present invention may be found in U.S. Pat. No. 5,567,831.

R¹ and R²

In one preferred aspect the compound is of Formula I

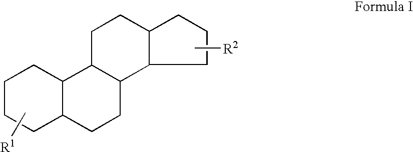

In one preferred aspect the compound is of Formula Ia

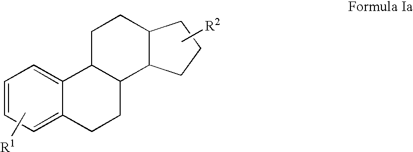

In one preferred aspect the compound is of Formula Ib

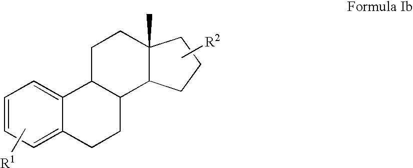

In one preferred aspect the compound is of Formula II

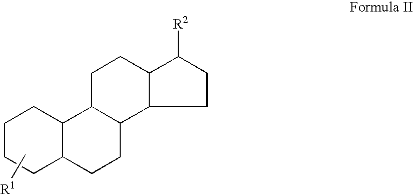

In one preferred aspect the compound is of Formula Ia

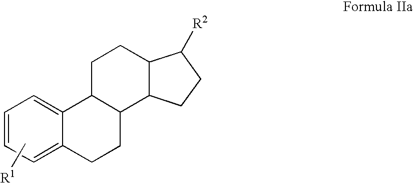

In one preferred aspect the compound is of Formula IIb

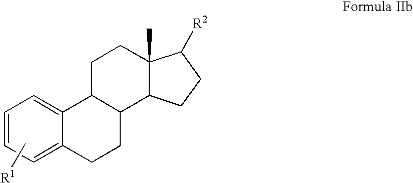

In one preferred aspect the compound is of Formula III

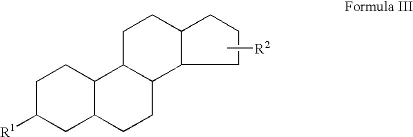

In one preferred aspect the compound is of Formula IIIa

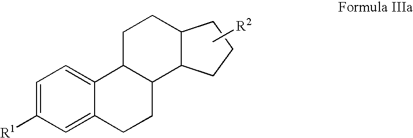

In one preferred aspect the compound is of Formula IIIb

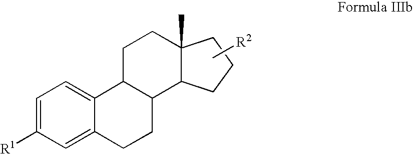

In one preferred aspect the compound is of Formula IVa or Formula IVb

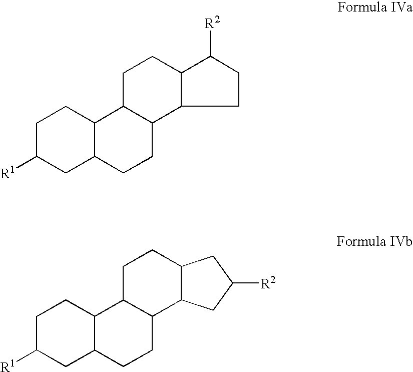

In one preferred aspect the compound is of Formula IVc or Formula IVd

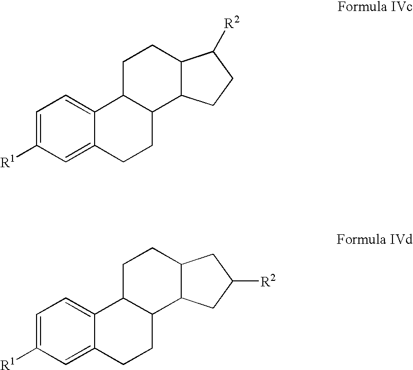

In one preferred aspect the compound is of Formula IVe or Formula IVf

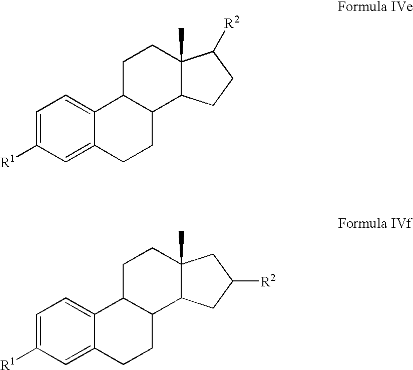

In one preferred aspect the compound is of Formula Va or Formula Vb

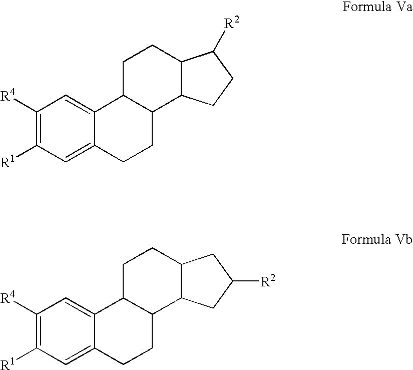

In one preferred aspect the compound is of Formula Vc or Formula Vd

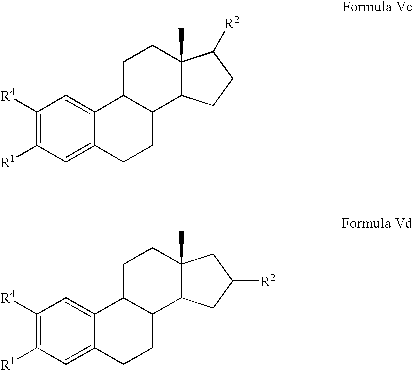

It will be appreciated by one skilled in the art that R¹ is an optional group which may or may not be present. In one preferred aspect R¹ is present. In this aspect R¹ is a group selected from any one of —OH, a sulphamate group, a phosphonate group, a thiophosphonate group, a sulphonate group or a sulphonamide group.

Sulphamate Group

In one aspect R¹ is an optional sulphamate group.

The term “sulphamate” includes an ester of sulphamic acid, or an ester of an N-substituted derivative of sulphamic acid, or a salt thereof.

In one aspect R¹ is a sulphamate group. In this aspect the compound of the present invention may be referred to as a sulphamate compound.

Preferably the sulphamate group of R¹, is a sulphamate group of the formula 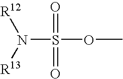
wherein R¹² and R¹³ are independently selected from H or a hydrocarbyl group.

Preferably R¹² and R¹³ are independently selected from H, alkyl, cycloalkyl, alkenyl, acyl and aryl, or combinations thereof, or together represent alkylene, wherein the or each alkyl or cycloalkyl or alkenyl or aryl optionally contains one or more hetero atoms or groups.

When substituted, the N-substituted compounds of this invention may contain one or two N-alkyl, N-alkenyl, N-cycloalkyl, N-acyl, or N-aryl substituents, preferably containing or each containing a maximum of 10 carbon atoms. When R¹² and/or R¹³ is alkyl, the preferred values are those where R¹² and R¹³ are each independently selected from lower alkyl groups containing from 1 to 5 carbon atoms, that is to say methyl, ethyl, propyl etc. Preferably R⁵ and R⁶ are both methyl. When R¹² and/or R¹³ is aryl, typical values are phenyl and tolyl (—PhCH₃; o-, m- or p-). Where R⁵ and R⁶ represent cycloalkyl, typical values are cyclopropyl, cyclopentyl, cyclohexyl etc. When joined together R¹² and R¹³ typically represent an alkylene group providing a chain of 4 to 6 carbon atoms, optionally interrupted by one or more hetero atoms or groups, e.g. —O— or —NH— to provide a 5-, 6- or 7-membered heterocycle, e.g. morpholino, pyrrolidino or piperidino.

Within the values alkyl, cycloalkyl, alkenyl, acyl and aryl we include substituted groups containing as substituents therein one or more groups which do not interfere with the sulphatase inhibitory activity of the compound in question. Exemplary non-interfering substituents include hydroxy, amino, halo, alkoxy, alkyl and aryl. A non-limiting example of a hydrocarbyl group is an acyl group.

In some embodiments, the sulphamate group may form a ring structure by being fused to (or associated with) one or more atoms in or on the steroidal ring system.

In some embodiments, there may be more than one sulphamate group. By way of example, there may be two sulphamates (i.e. bis-sulphamate compounds).

In some preferred embodiments, at least one of R¹² and R¹³ is H.

In some preferred embodiments, each of R¹² and R¹³ is H.

In some preferred embodiments R¹ is a sulphamate group and the compound is suitable for use as an inhibitor of oestrone sulphatase (E.C. 3.1.6.2).

In some preferred embodiments if the sulphamate group on the sulphamate compound were to be replaced with a sulphate group to form a sulphate compound then the sulphate compound would be hydrolysable by a steroid sulphatase enzyme (E.C.3.1.6.2).

In some preferred embodiments if the sulphamate group on the sulphamate compound were to be replaced with a sulphate group to form a sulphate compound and incubated with a steroid sulphatase enzyme (E.C.3.1.6.2) at a pH 7.4 and 37° C. it would provide a Kₘ value of less than 50 mM.

In some preferred embodiments if the sulphamate group on the sulphamate compound were to be replaced with a sulphate group to form a sulphate compound and incubated with a steroid sulphatase enzyme (E.C.3.1.6.2) at a pH 7.4 and 37° C. it would provide a Kₘ value of less than 50 μM.

Phosphonate Group

If the compound of the present invention comprises a phosphonate group then the compound of the present invention is referred to as a phosphonate compound.

Typically, the phosphonate group has the formula:
(R¹⁸)—P(O)(OH)—O—
wherein preferably R¹⁸ is H, alkyl, cycloalkyl, alkenyl, acyl or aryl, or combinations thereof, wherein the or each alkyl or cycloalkyl or alkenyl or aryl optionally contains one or more hetero atoms or groups.

When R¹⁸ is alkyl, R¹⁸ may be a lower alkyl groups containing from 1 to 6 carbon atoms, that is to say methyl, ethyl, propyl etc. By way of example, R¹⁸ may be methyl. When R¹³ is aryl, typical values are phenyl and tolyl (PhCH₃; o-, m-, p-). Where R¹⁸ represents cycloalkyl, typical values are cyclopropyl, cyclopentyl, cyclohexyl etc. R¹⁸ may even comprise an alkylene group providing a chain of 4 to 6 carbon atoms, optionally interrupted by one or more hetero atoms or groups, e.g. to provide a 5 membered heterocycle, e.g. morpholino, pyrrolidino or piperidino.

Within the values alkyl, cycloalkyl, alkenyl, acyl and aryl substituted groups are included containing as substituents therein one or more groups which do not interfere with the sulphatase inhibitory activity of the compound in question. Exemplary non-interfering substituents include hydroxy, amino, halo, alkoxy, alkyl and aryl.

In some embodiments, the phosphonate group may form a ring structure by being fused to (or associated with) one or more atoms in or on the steroidal ring system.

In some embodiments, there may be more than one phosphonate group. By way of example, there may be two phosphonates (i.e. bis-phosphonate compounds). These groups need not be the same.

Thiophosphonate Group

If the compound of the present invention comprises a thiophosphonate group then the compound of the present invention is referred to as a thiophosphonate compound.

Typically, the thiophosphonate group has the formula:
(R¹⁹)—P(S)(OH)—O—
wherein preferably R¹⁹ is H, alkyl, cycloalkyl, alkenyl, acyl or aryl, or combinations thereof, wherein the or each alkyl or cycloalkyl or alkenyl or aryl optionally contains one or more hetero atoms or groups.

When R¹⁹ is alkyl, R¹⁹ may be a lower alkyl groups containing from 1 to 6 carbon atoms, that is to say methyl, ethyl, propyl etc. By way of example, R¹⁹ may be methyl. When R¹⁹ is aryl, typical values are phenyl and tolyl (PhCH₃; o-, m-, p-). Where R¹⁹ represents cycloalkyl, typical values are cyclopropyl, cyclopentyl, cyclohexyl etc. R¹⁹ may even comprise an alkylene group providing a chain of 4 to 6 carbon atoms, optionally interrupted by one or more hetero atoms or groups, e.g. to provide a 5 membered heterocycle, e.g. morpholino, pyrrolidino or piperidino.

Within the values alkyl, cycloalkyl, alkenyl, acyl and aryl substituted groups are included containing as substituents therein one or more groups which do not interfere with the sulphatase inhibitory activity of the compound in question. Exemplary non-interfering substituents include hydroxy, amino, halo, alkoxy, alkyl and aryl.

In some embodiments, the thiophosphonate group may form a ring structure by being fused to (or associated with) one or more atoms in or on the steroidal ring system.

In some embodiments, there may be more than one thiophosphonate group. By way of example, there may be two thiophosphonates (i.e. bis-thiophosphonate compounds). These groups need not be the same.

Sulphonate Group

If the compound of the present invention comprises a sulphonate group then the compound of the present invention is referred to as a sulphonate compound.

Typically, the sulphonate group has the formula:
(R²⁰)—S(O)(O)—O—
wherein preferably R²⁰ is H, alkyl, cycloalkyl, alkenyl, acyl or aryl, or combinations thereof, wherein the or each alkyl or cycloalkyl or alkenyl or aryl optionally contains one or more hetero atoms or groups.

When R²⁰ is alkyl, R²⁰ may be a lower alkyl groups containing from 1 to 6 carbon atoms, that is to say methyl, ethyl, propyl etc. By way of example, R²⁰ may be methyl. When R²⁰ is aryl, typical values are phenyl and tolyl (PhCH₃; o-, m-, p-). Where R²⁰ represents cycloalkyl, typical values are cyclopropyl, cyclopentyl, cyclohexyl etc. R²⁰ may even comprise an alkylene group providing a chain of 4 to 6 carbon atoms, optionally interrupted by one or more hetero atoms or groups, e.g. to provide a 5 membered heterocycle, e.g. morpholino, pyrrolidino or piperidino.

Within the values alkyl, cycloalkyl, alkenyl, acyl and aryl substituted groups are included containing as substituents therein one or more groups which do not interfere with the sulphatase inhibitory activity of the compound in question. Exemplary non-interfering substituents include hydroxy, amino, halo, alkoxy, alkyl and aryl.

In some embodiments, the sulphonate group may form a ring structure by being fused to (or associated with) one or more atoms in or on the steroidal ring system.

In some embodiments, there may be more than one sulphonate group. By way of example, there may be two sulphonates (i.e. bis-sulphonate compounds). These groups need not be the same.

Other Substituents

The compound of the present invention may have substituents other than those of formula I. By way of example, these other substituents may be one or more of: one or more sulphamate group(s), one or more phosphonate group(s), one or more thiophosphonate group(s), one or more sulphonate group(s), one or more sulphonamide group(s), one or more halo groups, one or more 0 groups, one or more hydroxy groups, one or more amino groups, one or more sulphur containing group(s), one or more hydrocarbyl group(s)—such as an oxyhydrocarbyl group.

R²

The D ring of the steroidal ring system of the present compound is substituted by a group R² of the formula -L-R³, wherein L is an optional linker group and R³ selected from groups which are or which comprise one of (i) —SO₂R⁵, wherein R⁵ is H, a hydrocarbyl group or a bond or group attached to the D ring (ii) —NO₂ (iii) —SOR⁶, wherein R⁶ is H or a hydrocarbyl group (iv) —R⁷, wherein R⁷ is a halogen (v) -alkyl (vi) —C(═O)R⁶, wherein R⁸ is H or hydrocarbyl (vii) —C≡CR⁹, wherein R⁹ is H or hydrocarbyl (viii) —OC(═O)NR¹⁰R¹¹, wherein R¹⁰ and R¹¹ are independently selected from H and hydrocarbyl

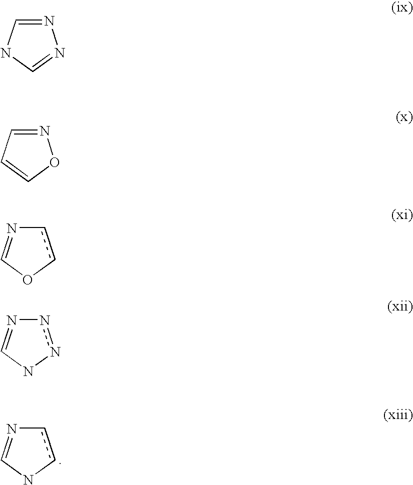

In some preferred embodiments R² is of the formula —R³, In other words no group L is present.

In some preferred aspects group R² is in an a configuration. Preferably group R² is in an a configuration on the 17 position of the D ring.

L

In some embodiments L is selected from a hydrocarbyl group, —NR¹⁴— and —O—, wherein R¹⁴ is H, a hydrocarbyl group or a bond.

Preferably L is selected from a hydrocarbon group, —NR¹⁴— and —O—.

In one aspect L is selected from an alkylene group (such as C₁₋₁₀ alkylene, a C₁₋₅ alkylene, a C₁ or C₂ alkylene), —NR¹⁴— and —O—.

In one aspect L is selected from a C₁₋₁₀ alkylene group, —NR¹⁴— and —O—.

In one aspect L is selected from a C₁ or C₂ alkylene group, —NR¹⁴— and —O—.

Particularly preferred linkers are ═N—, —NH—, ═CH—, —CH₂—, —CH₂CH₂— and ═CHCH₂—, such as ═N—, —NH—, ═CH—, and —CH₂—.

R³

As discussed above R³ is selected from (i) —SO₂R⁵, wherein R⁵ is H, a hydrocarbyl group or a bond or group attached to the D ring (ii) —NO₂ (iii) —SOR⁶, wherein R⁶ is H or a hydrocarbyl group (iv) —R⁷, wherein R⁷ is a halogen (v) -alkyl (vi) —C(═O)R⁸, wherein R⁸ is H or hydrocarbyl (vii) —C≡CR⁹, wherein R⁹ is H or hydrocarbyl (viii) —OC(═O)NR¹⁰R¹¹, wherein R¹⁰ and R¹¹ are independently selected from H and hydrocarbyl

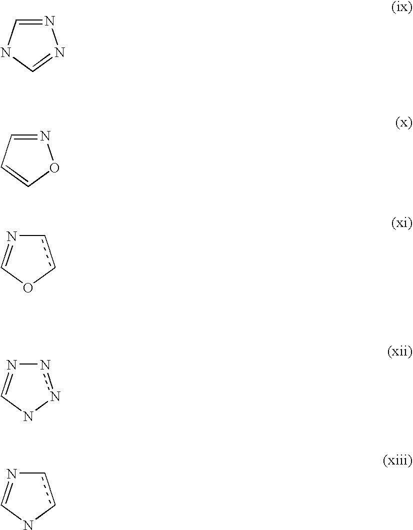

R³ may be a cyclic group or an acyclic group.

When R³ is a cyclic group is may form a ring which is fused with the D ring of the steroid or which is not fused with the D ring of the steroid. When R³ forms a cyclic group which is fused with the D ring of the steroid, preferably R³ forms a ring joining adjacent members of the D ring, more preferably R³ forms a ring joining positions 16 and 17 of the D ring.

It will be appreciated by one skilled in the art that group R³ may be attached to optional L at any point on R³. Preferred points of attachment are shown when groups (ix) to (xiiii) are selected from optionally substituted groups of the formulae 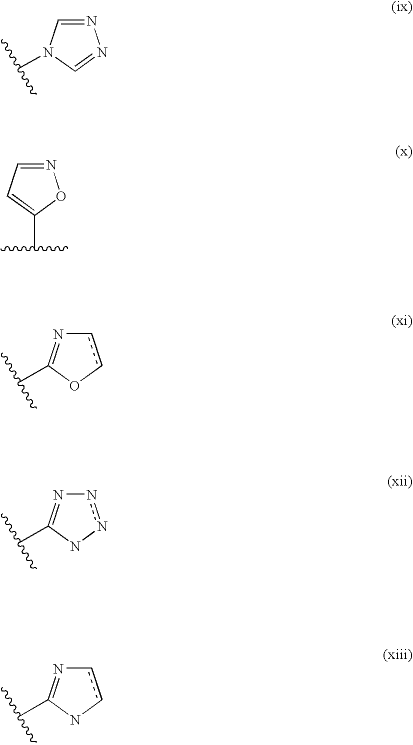
—SO₂R⁵

In one preferred aspect R³ is —SO₂R⁵, wherein R⁵ is H, a hydrocarbyl group or a bond or group attached to the D ring.

Preferably R⁵ is selected from H and hydrocarbyl. In one aspect R⁵ is hydrocarbyl. In one preferred embodiment of the present invention R⁵ is selected from one of H, C₁-C₂₀ hydrocarbyl, C₁-C₁₀ hydrocarbyl, C₁-C₆ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl.

In one aspect R⁵ is selected from H and C₁₋₁₀ alkyl. In one aspect R⁵ is C₁₋₁₀ alkyl. In one aspect R⁵ is selected from H and C₁₋₅ alkyl. In one aspect R⁵ is C₁₋₅ alkyl. In one aspect R⁵ is selected from H and C₁₋₃ alkyl. In one aspect R⁵ is C₁₋₃ alkyl. Preferably R⁵ is —CH₃.

Preferably R⁵ is —O—R¹⁵-D, wherein R¹⁵ is a linker and D is a member of the D ring. In a preferred aspect this provides a compound of the formula

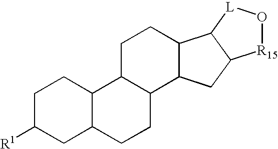

R¹⁵ may be any suitable group. Particularly preferred are —O—CH₂— and —N═CH—

In this aspect preferably R² is —CH₂—R³ or —NH—R³, for example in one preferred aspect R² is —NH—SO₂—CH₃.

—NO₂

In one preferred aspect wherein R³ is —NO₂

In this aspect preferably R² is —CH₂—R³

—SOR⁶

In one preferred aspect R³ is —SOR⁶, wherein R⁶ is H or a hydrocarbyl group.

Preferably R⁶ is selected from H and hydrocarbyl. In one aspect R⁶ is hydrocarbyl. In one preferred embodiment of the present invention R⁶ is selected from one of H, C₁-C₂₀ hydrocarbyl, C₁-C₁₀ hydrocarbyl, C₁-C₅ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl.

In one aspect R⁶ is selected from H and C₁₋₁₀ alkyl. In one aspect R⁶ is C₁₋₁₀ alkyl. In one aspect R⁶ is selected from H and C₁₋₅ alkyl. In one aspect R⁶ is C₁₋₅ alkyl. In one aspect R⁶ is selected from H and C₁₋₃ alkyl. In one aspect R⁶ is C₁₋₃ alkyl. Preferably R⁶ is —CH₃.

In this aspect preferably R² is —CH₂—R³

R⁷

In one preferred aspect R³ is —R⁷, wherein R⁷ is a halogen

It will be appreciated that R⁷ may chlorine, fluorine, bromine or iodine. Preferably R⁷ is fluorine.

In this aspect preferably R² is —CH₂CH₂—R³, namely —CH₂CH₂—R⁷.

In this aspect preferably R² is —CH₂CHX—R⁷ wherein X is a halogen. For example X may be F and R⁷ may be F such that R² is —CH₂CF₂H.

In this aspect R² may also be —CX₂—R³, wherein each X is independently selected from halogens. For example each X may be F and R³ may be F such that R² is CF₃.

In this aspect R² may be —CY₂—R³ or —CY₂CY₂—R³, wherein each Y is independently selected from H and halogens. For example one or more Y may be F and R³ may be F. When only one Y is H and the remaining Y are H, R² may be —CHY—R³ or —CH₂CHY—R³, wherein Y is selected from H and halogens. For example Y may be F and R³ may be F.

-alkyl

In one preferred aspect R³ is -alkyl

In one preferred embodiment of the present invention R³ is selected from one of C₁-C₂₀ alkyl, C₁-C₁₀ alkyl, C₁-C₅ alkyl, and C₁-C₃ alkyl.

In one aspect R³ is C₁₋₁₀ alkyl. In one aspect R³ is C₁₋₅ alkyl. In one aspect R³ is C₁ alkyl. Preferably R³ is —CH₃ or —CH₂CH₃.

In this aspect preferably R² is R³.

In one preferred aspect when R³ is alkyl the compound is of Formula IVb

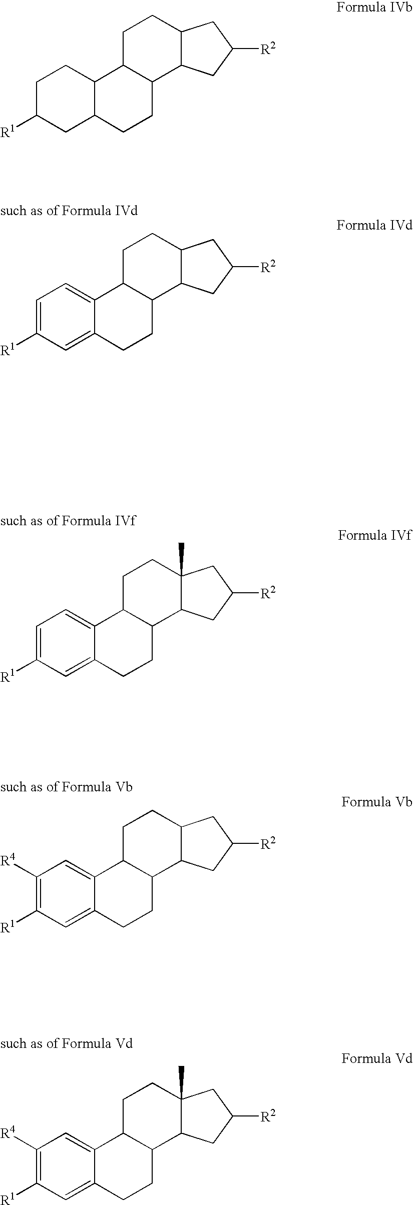

In one aspect the compound further comprises a further group denoted R², which is an alkyl group and preferably an alkyl group described under (v) herein. Thus in one preferred aspect the compound is selected from compounds of the formulae 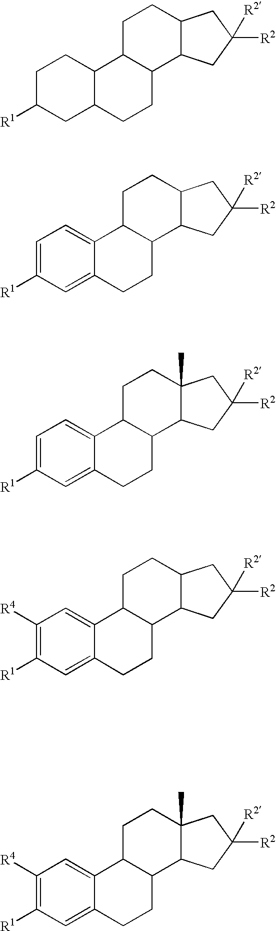
wherein R² and R²′ are independently selected from one of C₁-C₂₀ hydrocarbyl, C₁-C₁₀ hydrocarbyl, C₁-C₈ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl. In a highly preferred aspect each of R² and R²′ are —CH₃.
—C(═O)R⁸

In one preferred aspect R³ is —C(═O)R⁸, wherein R⁸ is H or hydrocarbyl

Preferably R⁸ is selected from H and hydrocarbyl. In one aspect R⁸ is hydrocarbyl. In one preferred embodiment of the present invention R⁸ is selected from one of H, C₁-C₂₀ hydrocarbyl, C₁-C₁₀ hydrocarbyl, C₁-C₅ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl.

In one aspect R² is selected from H and C₁₋₁₀ alkyl. In one aspect R⁸ is C₁₋₁₀ alkyl. In one aspect R⁸ is selected from H and C₁₋₅ alkyl. In one aspect R⁹ is C₁₋₅ alkyl. In one aspect R⁸ is selected from H and C₁₋₃ alkyl. In one aspect R⁸ is C₁₋₃ alkyl. Preferably R⁸ is —CH₃.

In this aspect preferably R² is —CH₂—R³ or R³, for example —C(═O)CH₃.

—C≡CR⁹

In one preferred aspect R³ is —C≡CR⁹, wherein R⁹ is H or hydrocarbyl

Preferably R⁹ is selected from H and hydrocarbyl. In one aspect R⁹ is hydrocarbyl. In one preferred embodiment of the present invention R⁹ is selected from one of H, C₁-C₂₀ hydrocarbyl, C₁-C₁₀ hydrocarbyl, C₁-C₅ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl.

In one aspect R⁹ is selected from H and C₁₋₁₀ alkyl. In one aspect R⁹ is C₁₋₁₀ alkyl. In one aspect R⁹ is selected from H and C₁₋₅ alkyl. In one aspect R⁹ is C₁₋₅ alkyl. In one aspect R⁹ is selected from H and C₁₋₃ alkyl. In one aspect R⁹ is C₁₋₃ alkyl. Preferably R⁹ is —CH₃.

In this aspect preferably R² is —CH₂—R³

—OC(═O)NR¹⁰R¹¹

In one preferred aspect R³ is —OC(═O)NR¹⁰R¹¹, wherein R¹⁰ and R¹¹ are independently selected from H and hydrocarbyl

Preferably R¹⁰ and R¹¹ are independently selected from H and hydrocarbyl. In one aspect R¹⁰ and R¹¹ are independently selected from hydrocarbyl. In one preferred embodiment of the present invention R¹⁰ and R¹¹ are independently selected from one of H, C₁-C₂₀ hydrocarbyl, C₁-C₁₀ hydrocarbyl, C₁-C₅ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl.

In one aspect R¹⁰ and R¹¹ are independently selected from H and C₁₋₁₀ alkyl. In one aspect R¹⁰ and R¹¹ are independently selected from C₁₋₁₀ alkyl. In one aspect R¹⁰ and R¹¹ are independently selected from H and C₁₋₅ alkyl. In one aspect R¹⁰ and R¹¹ are independently selected from C₁₋₅ alkyl. In one aspect R¹⁰ and R¹¹ are independently selected from H and C₁₋₃ alkyl. In one aspect R¹⁰ and R¹¹ are independently selected from C₁₋₃ alkyl. Preferably R¹⁰ and R¹¹ are both H.

In this aspect preferably R² is R³.

Cyclic Groups

In one preferred aspect R³ is

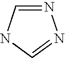

Preferably R³ is

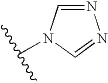

In this aspect preferably R² is selected from —CH₂CH₂—R³, ═N—R³ and —NH—R³

In one preferred aspect wherein R³ is

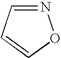

Preferably R³ is

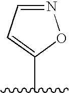

Preferably R³ is

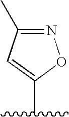

In this aspect preferably R² is selected from ═CH—R³ and —CH₂CH₂—R³

In one preferred aspect wherein R³ is

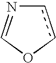

Preferably R³ is

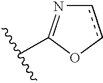

In this aspect preferably R² is selected from ═CH—R³ and —CH₂CH₂—R³

In one preferred aspect R³ is

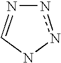

Preferably R³ is

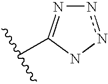

Preferably R³ is selected from

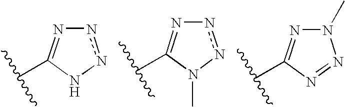

In this aspect preferably R² is selected from ═CH—R³ and —CH₂CH₂—R³

In one preferred aspect R³ is

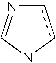

Preferably R³ is

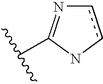

In this aspect preferably R² is selected from ═CH—R³ and —CH₂CH₂—R³

R⁴

As previously mentioned, the A ring of the steroidal ring system is optionally substituted with a group R⁴, wherein R⁴ is preferably selected from a hydrocarbyl group or an oxyhydrocarbyl group.

In one preferred embodiment of the present invention, the R⁴ is a oxyhydrocarbon group.

Here the term “oxyhydrocarbon” means, or R⁴ is, any one of an alkoxy group, an oxyalkenyl group, an oxyalkynyl group, which groups may be linear, branched or cyclic, or an oxyaryl group. The term oxyhydrocarbon also includes those groups but wherein they have been optionally substituted. If the oxyhydrocarbon is a branched structure having substituent(s) thereon, then the substitution may be on either the hydrocarbon backbone or on the branch; alternatively the substitutions may be on the hydrocarbon backbone and on the branch.

Preferably the oxyhydrocarbyl group R⁴ is an alkoxy group. Preferably the oxyhydrocarbyl group R⁴ is of the formula C₁₀ (such as a C₁₋₃O). Preferably the oxyhydrocarbyl group R⁴ is of the formula —O(CH₂)₁₋₁₀CH₃, —O(CH₂)₁₋₅CH₃, —O(CH₂)₁₋₂CH₃. In a highly preferred aspect R⁴ is methoxy.

Preferably the oxyhydrocarbyl group R⁴ is an ether group. Preferably the oxyhydrocarbyl group R⁴ is of the formula C₁₋₆OC₁₋₆ (such as a C₁₋₃OC₁₋₃). Preferably the oxyhydrocarbyl group R⁴ is of the formula —(CH₂)₁₋₁₀—O(CH₂)₁₋₁₀CH₃, —(CH₂)₁₋₅O(CH₂)₁₋₅CH₃, —(CH₂)₁₋₂—O(CH₂)₁₋₂CH₃. In a highly preferred aspect R⁴ is —CH₂OCH₃.

In one preferred embodiment of the present invention, R⁴ is a hydrocarbon group. Preferably R⁴ is an alkyl group. Preferably the alkyl group is a C₁₋₈ alkyl group (such as a C₁₋₃ alkyl group). Preferably the hydrocarbyl group R⁴ is of the formula —(CH₂)₁₋₁₀CH₃, —(CH₂)₁₋₅CH₃, —(CH₂)₁₋₂CH₃. In a highly preferred aspect R⁴ is ethyl.

In one preferred embodiment of the present invention R⁴ is selected from one of C₁-C₁₀ hydrocarbyl, C₁-C₅ hydrocarbyl, C₁-C₃ hydrocarbyl, hydrocarbon groups, C₁-C₁₀ hydrocarbon, C₁-C₅ hydrocarbon, C₁-C₃ hydrocarbon, alkyl groups, C₁-C₁₀ alkyl, C₁-C₅ alkyl, and C₁-C₃ alkyl.

In one preferred embodiment of the present invention, the R⁴ is a hydrocarbylsulphanyl group.

The term “hydrocarbylsulphanyl” means a group that comprises at least hydrocarbyl group (as herein defined) and sulphur. That sulphur group may be optionally oxidised. Preferably the hydrocarbylsulphanyl is of the formula —S-hydrocarbyl wherein the hydrocarbyl is as described herein.

The term “hydrocarbylsulphanyl group” as used herein with respect to R⁴ means a group comprising at least C, H and S and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbylsulphanyl group comprises more than one C then those carbons need not necessarily be linked to each other. For example, at least two of the carbons may be linked via a suitable element or group. Thus, the hydrocarbylsulphanyl group may contain further hetero atoms. Suitable hetero atoms will be apparent to those skilled in the art and include, for instance, nitrogen.

In one preferred embodiment of the present invention, the R⁴ is a hydrocarbonsulphanyl group. The term “hydrocarbonsulphanyl group” as used herein with respect to R⁴ means a group consisting of C, H and S. Preferably the hydrocarbonsulphanyl is of the formula —S— hydrocarbon wherein the hydrocarbon is as described herein.

Preferably the hydrocarbonsulphanyl group R⁴ is of the formula C₁₋₆S (such as a C₁₋₃S). Preferably the oxyhydrocarbyl group R⁴ is of the formula —S(CH₂)₁₋₁₀CH₃, —S(CH₂)₁₋₅CH₃, —S(CH₂)₁₋₂CH₃. In a highly preferred aspect R⁴ is —S-Me.

As previously mentioned, R⁴ is at position 2 or 4 of the A ring. Thus the compound may have the formula 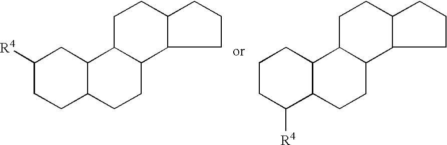
wherein R¹ and R² are as specified herein, such as

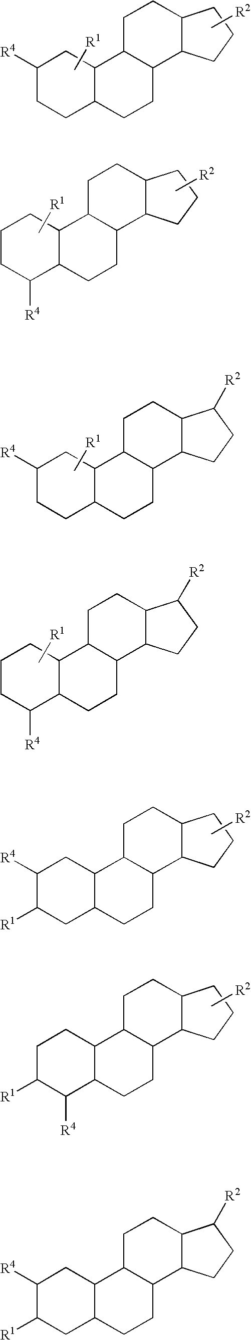

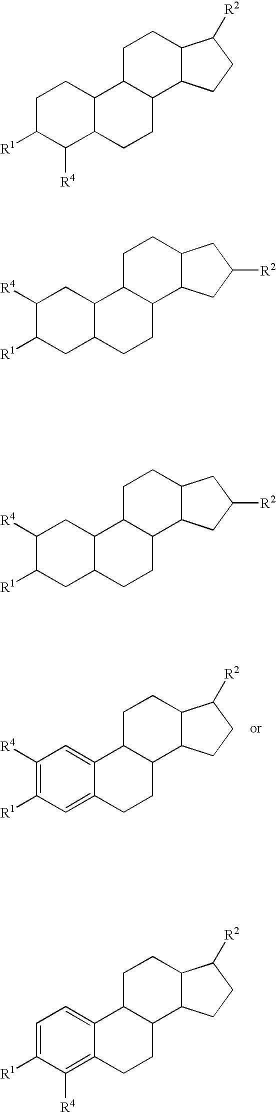

Preferably R⁴ is at position 2 of the A ring.

In a further preferred aspect when the A ring is substituted with R¹ and R⁴, R⁴ is ortho with respect to R¹.

It will be appreciated by one skilled in that the proviso that R⁴ is at position 2 or 4 of the A ring, allows for R⁴ being at position 2 and 4 of the A ring, wherein each R⁴ is independently selected from the possibilities recited herein.

Highly preferred compounds of the present invention are compounds 7, 8, 9, 10, 11, 13, 14, 19, 20, 22, 23, 28, 29, 32, 33, 34, 35, 36, 37, 40, 41, 43, 44, 46, 47, 50, 51, 55, 56, 57 and 58 of the experimental section below.

Further Aspects

In one preferred aspect R² is in the β configuration on the D ring. We have found that this configuration provides particularly good activity. Indeed we have found that when R³ is a cyclic structure β configuration is particularly preferred. this novel finding applies to all cyclic systems.

Thus in a further aspect (the “aspect”) the present invention provides
a compound comprising a steroidal ring system and an optional group R¹ selected from any one of —OH, a sulphamate group, a phosphonate group, a thiophosphonate group, a sulphonate group or a sulphonamide group; wherein the D ring of the steroidal ring system is substituted by a group R² of the formula -L-R³, wherein L is an optional linker group and R³ is a cyclic group, and wherein R² is in the β configuration on the D ring.
a pharmaceutical composition comprising (a) a compound as defined herein and (b) a pharmaceutically acceptable carrier, diluent, excipient or adjuvant.
a (i) compound as defined herein, or (ii) composition as defined herein, for use in medicine
use of (i) a compound as defined herein, or (ii) a composition as defined herein, in the manufacture of a medicament to prevent and/or inhibit tumour growth.
use of (i) a compound as defined herein, or (ii) a composition as defined herein, in the manufacture of a medicament for use in the therapy of a condition or disease associated with one or more of steroid sulphatase (STS) activity; cell cycling; apoptosis; cell growth; glucose uptake by a tumour; tumour angiogenesis; microtubules formation; and apoptosis.
use of (i) a compound as defined herein, or (ii) a composition as defined herein, in the manufacture of a medicament for use in the therapy of a condition or disease associated with one or more of adverse steroid sulphatase (STS) activity; cell cycling; apoptosis; cell growth; glucose uptake by a tumour; tumour angiogenesis; microtubules formation; and apoptosis.
use of (i) a compound as defined herein, or (ii) a composition as defined herein, in the manufacture of a medicament for one or more of inhibiting steroid sulphatase (STS) activity; modulating cell cycling; modulating apoptosis; modulating cell growth; preventing and/or suppressing glucose uptake by a tumour; preventing and/or inhibiting tumour angiogenesis; disrupting microtubules; and inducing apoptosis.
use of (i) a compound as defined herein, or (ii) a composition as defined herein, in the manufacture of a medicament for inhibiting steroid sulphatase (STS) activity.
use of (i) a compound as defined herein, or (ii) a composition as defined herein, in the manufacture of a medicament for modulating cell growth.
a method of treatment comprising administering to a subject in need of treatment (i) a compound as defined herein, or (ii) a composition as defined herein.
a method of treatment comprising administering to a subject in need of treatment (i) a compound as defined herein, or (ii) a composition as defined herein, in order to inhibit steroid sulphatase (STS) activity; modulate cell cycling; modulate apoptosis; modulate cell growth; prevent and/or suppress glucose uptake by a tumour; prevent and/or inhibit tumour angiogenesis; disrupt microtubules; and/or induce apoptosis.
a method comprising (a) performing an assay for one or more of steroid sulphatase (STS) inhibition; cell cycling modulation; apoptosis modulation; cell growth modulation; prevention and/or suppression of glucose uptake by a tumour; tumour angiogenesis prevention and/or inhibition; microtubules disruption; and apoptosis induction, with one or more candidate compounds defined herein; (b) determining whether one or more of said candidate compounds is/are capable of one or more of steroid sulphatase (STS) inhibition; cell cycling modulation; apoptosis modulation; cell growth modulation; prevention and/or suppression of glucose uptake by a tumour; tumour angiogenesis prevention and/or inhibition; microtubules disruption; and apoptosis induction; and (c) selecting one or more of said candidate compounds that is/are capable of one or more of steroid sulphatase (STS) inhibition; cell cycling modulation; apoptosis modulation; cell growth modulation; prevention and/or suppression of glucose uptake by a tumour; tumour angiogenesis prevention and/or inhibition; microtubules disruption; and apoptosis induction.

In the β aspect, preferably the R2 group is attached to the 17 position of the steroid.

In the β aspect R³ may be an aromatic hydrocarbyl group. The term “aromatic hydrocarbyl group” used herein means any hydrocarbyl group which contains or form part of a ring system containing delocalised π electrons.

Preferably in the β aspect R³ is or comprises an aromatic ring. Preferably R³ is an optionally substituted aromatic ring

Preferably in the β aspect R³ is a heterocyclic group, that is a ring containing carbon and at least one other atom. Suitable hetero atoms will be apparent to those skilled in the art and include, for instance, sulphur, nitrogen and oxygen.

Preferably in the β aspect R³ is or comprises a aromatic ring containing carbon and optionally nitrogen. Preferably R³ is an optionally substituted aromatic ring containing carbon and optionally nitrogen.

Preferably R³ is or comprises a five or six membered aromatic ring. Preferably R³ is an optionally substituted five or six membered aromatic ring.

Preferably R³ is or comprises a five or six membered aromatic ring containing carbon and optionally nitrogen. Preferably R³ is an optionally substituted five or six membered aromatic ring containing carbon and optionally nitrogen.

Preferably in the β aspect R³ is as defined herein

In these broad aspects, preferably R¹ to R¹¹ and L are as herein defined.

In one preferred aspect R⁵ of the group may be selected from H, a hydrocarbyl group, a bond or group attached to the D ring, and a group of the formula NR²¹R²², wherein R²¹ and R²² are independently selected from H and hydrocarbyl. Thus in a further aspect the present invention provides
a compound comprising a steroidal ring system and an optional group R¹ selected from any one of —OH, a sulphamate group, a phosphonate group, a thiophosphonate group, a sulphonate group or a sulphonamide group; wherein the A ring of the steroidal ring system is optionally substituted at position 2 or 4 with a group R⁴ which may be a suitable subtituent wherein the D ring of the steroidal ring system is substituted by a group R² of the formula -L-R³, wherein L is an optional linker group and R³ is selected from groups which are or which comprise one of (i) —SO₂R⁵, wherein R⁵ is H, a hydrocarbyl group, a bond or group attached to the D ring and a group of the formula NR²¹R²², wherein R²¹ and R²² are independently selected from H and hydrocarbyl (ii) —NO₂ (iii) —SOR⁶, wherein R⁶ is H or a hydrocarbyl group (iv) —R⁷, wherein R⁷ is a halogen (v) -alkyl (vi) —C(═O)R⁸, wherein R⁸ is H or hydrocarbyl (vii) —C≡CR⁹, wherein R⁹ is H or hydrocarbyl (viii) —OC(═O)NR¹⁰R¹¹, wherein R¹⁰ and R¹¹ are independently selected from H and hydrocarbyl 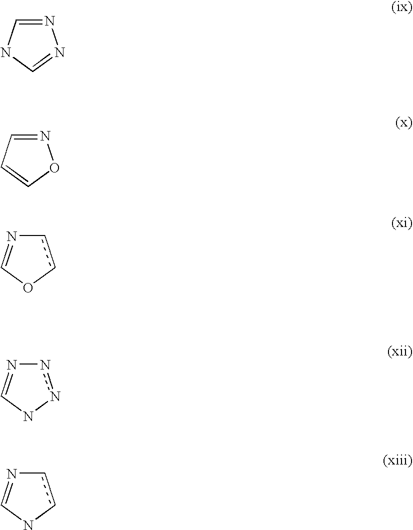
wherein when R³ is -alkyl, R⁴ is present as a hydrocarbon group, when R³ is —NO₂R⁴ is present and/or R¹ is present as a sulphamate group, and when R³ is —C(═O)R⁸R⁴ is present and R¹ is present as a sulphamate group.
a pharmaceutical composition comprising (a) a compound as defined herein and (b) a pharmaceutically acceptable carrier, diluent, excipient or adjuvant.
a (i) compound as defined herein, or (ii) composition as defined herein, for use in medicine
use of (i) a compound as defined herein, or (ii) a composition as defined herein, in the manufacture of a medicament to prevent and/or inhibit tumour growth.
use of (i) a compound as defined herein, or (ii) a composition as defined herein, in the manufacture of a medicament for use in the therapy of a condition or disease associated with one or more of steroid sulphatase (STS) activity; cell cycling; apoptosis; cell growth; glucose uptake by a tumour; tumour angiogenesis; microtubules formation; and apoptosis.
use of (i) a compound as defined herein, or (ii) a composition as defined herein, in the manufacture of a medicament for use in the therapy of a condition or disease associated with one or more of adverse steroid sulphatase (STS) activity; cell cycling; apoptosis; cell growth; glucose uptake by a tumour; tumour angiogenesis; microtubules formation; and apoptosis.
use of (i) a compound as defined herein, or (ii) a composition as defined herein, in the manufacture of a medicament for one or more of inhibiting steroid sulphatase (STS) activity; modulating cell cycling; modulating apoptosis; modulating cell growth; preventing and/or suppressing glucose uptake by a tumour; preventing and/or inhibiting tumour angiogenesis; disrupting microtubules; and inducing apoptosis.
use of (i) a compound as defined herein, or (ii) a composition as defined herein, in the manufacture of a medicament for inhibiting steroid sulphatase (STS) activity.
use of (i) a compound as defined herein, or (ii) a composition as defined herein, in the manufacture of a medicament for modulating cell growth.
a method of treatment comprising administering to a subject in need of treatment (i) a compound as defined herein, or (ii) a composition as defined herein.
a method of treatment comprising administering to a subject in need of treatment (i) a compound as defined herein, or (ii) a composition as defined herein, in order to inhibit steroid sulphatase (STS) activity; modulate cell cycling; modulate apoptosis; modulate cell growth; prevent and/or suppress glucose uptake by a tumour; prevent and/or inhibit tumour angiogenesis; disrupt microtubules; and/or induce apoptosis.
a method comprising (a) performing an assay for one or more of steroid sulphatase (STS) inhibition; cell cycling modulation; apoptosis modulation; cell growth modulation; prevention and/or suppression of glucose uptake by a tumour; tumour angiogenesis prevention and/or inhibition; microtubules disruption; and apoptosis induction, with one or more candidate compounds defined herein; (b) determining whether one or more of said candidate compounds is/are capable of one or more of steroid sulphatase (STS) inhibition; cell cycling modulation; apoptosis modulation; cell growth modulation; prevention and/or suppression of glucose uptake by a tumour; tumour angiogenesis prevention and/or inhibition; microtubules disruption; and apoptosis induction; and (c) selecting one or more of said candidate compounds that is/are capable of one or more of steroid sulphatase (STS) inhibition; cell cycling modulation; apoptosis modulation; cell growth modulation; prevention and/or suppression of glucose uptake by a tumour; tumour angiogenesis prevention and/or inhibition; microtubules disruption; and apoptosis induction.

Preferably R²¹ and R²² are independently selected from H and hydrocarbyl. In one aspect R²¹ and R²² are independently selected from hydrocarbyl. In one preferred embodiment of the present invention R²¹ and R²² are independently selected from one of H, C₁-C₂₀ hydrocarbyl, C₁-C₁₀ hydrocarbyl, C₁-C₅ hydrocarbyl, C₁-C₃ hydrocarbyl, hydrocarbon groups, C₁-C₂₀ hydrocarbon, C₁-C₁₀ hydrocarbon, C₁-C₅ hydrocarbon, C₁-C₃ hydrocarbon, alkyl groups, C₁-C₂₀ alkyl, C₁-C₁₀ alkyl, C₁-C₅ alkyl, and C₁-C₃ alkyl.

In one aspect R²¹ and R²² are independently selected from H and C₁₋₁₀ alkyl. In one aspect R²¹ and R²² are independently selected from C₁₋₁₀ alkyl. In one aspect R²¹ and R²² are independently selected from H and C₁₋₅ alkyl. In one aspect R²¹ and R²² are independently selected from C₁₋₅ alkyl. In one aspect R²¹ and R²² are independently selected from H and C₁₋₃ alkyl. In one aspect R21R²¹ and R²² are independently selected from C₁₋₃ alkyl. Preferably R²¹ and R²² are both H.

Composition

As described above according to one aspect of the present invention, there is provided a pharmaceutical composition comprising (a) (i) a compound as defined herein, or (ii) a composition as defined herein, and (b) a pharmaceutically acceptable carrier, diluent, excipient or adjuvant.

In accordance with the present invention the composition of the present invention may comprise more than one biological response modifier.

The term biological response modifier (“BRM”) includes cytokines, immune modulators, growth factors, haematopoiesis regulating factors, colony stimulating factors, chemotactic, haemolytic and thrombolytic factors, cell surface receptors, ligands, leukocyte adhesion molecules, monoclonal antibodies, preventative and therapeutic vaccines, hormones, extracellular matrix components, fibronectin, etc.

BRMs may play a role in modulating the immune and inflammatory response in disorders. Examples of BRMs include: Tumour Necrosis Factor (TNF), granulocyte colony stimulating factor, erythropoietin, insulin-like growth factor (IGF), epidermal growth factor (EGF), transforming growth factor (TGF), platelet-derived growth factor (PDGF), interferons (IFNs), interleukins, tissue plasminogen activators, P-, E- or L-Selectins, ICAM-1, VCAM, Selectins, addressins etc.

Preferably, the biological response modifier is a cytokine.

A cytokine is a molecule—often a soluble protein—that allows immune cells to communicate with each other. These molecules exert their biological functions through specific receptors expressed on the surface of target cells. Binding of the receptors triggers the release of a cascade of biochemical signals which profoundly affect the behaviour of the cell bearing the receptor (Poole, S 1995 TibTech 13: 81-82). Many cytokines and their receptors have been identified at the molecular level (Paul and Sedar 1994, Cell 76: 241-251) and make suitable molecules of therapeutic value as well as therapeutic targets in their own right.

More details on cytokines can be found in Molecular Biology and Biotechnology (Pub. VCH, Ed. Meyers, 1995, pages 202, 203, 394, 390, 475, 790).

Examples of cytokines include: interleukins (IL)—such as IL-1, IL-2, IL-3, IL-4, IL-5, IL-6, IL-7, IL-8, IL-9, IL-10, IL-11, IL-12, IL-19; Tumour Necrosis Factor (TNF)—such as TNF-α; Interferon alpha, beta and gamma; TGF-β.

For the present invention, preferably the cytokine is tumour necrosis factor (TNF).

More preferably the cytokine is TNF-α.

TNF is a cytokine produced by macrophages and lymphocytes which mediates inflammatory and immunopathological responses. TNF has been implicated in the progression of diseases which include but are not limited to immunomodulation disorder, infection, cell proliferation, angiogenesis (neovascularisation), tumour metastasis, apoptosis, sepsis, and endotoxaemia.

The necrotising action of TNF in vivo mainly relates to capillary injury. TNF causes necrosis not only in tumour tissue but also in granulation tissue. It causes morphological changes in growth inhibition of and cytoxicity against cultured vascular endothelial cells (Haranka et al 1987 Ciba Found Symp 131: 140-153).

For the preferred aspect of the present invention, the TNF may be any type of TNF—such as TNF-α, TNF-β, including derivatives or mixtures thereof.

Teachings on TNF may be found in the art—such as WO-A-98/08870 and WO-A-98/13348.

The TNF can be prepared chemically or it can be extracted from sources. Preferably, the TNF is prepared by use of recombinant DNA techniques.

With this aspect of the present invention the compositions of the present invention are more potent in vivo than the compounds alone or TNF alone. Moreover, in some aspects the combination of compounds and TNF is more potent than one would expect from the potency of the compound alone i.e. this is a synergistic relationship between them.

In addition, the present invention contemplates the composition of the present invention further comprising an inducer of the biological response modifier—such as in vivo inducer of the biological response modifier.

In accordance with the present invention, the components of the composition can be added in admixture, simultaneously or sequentially. Furthermore, in accordance with the present invention it may be possible to form at least a part of the composition in situ (such as in vivo) by inducing the expression of—or increasing the expression of—one of the components. For example, it may be possible to induce the expression of—or increase the expression of—the biological response modifier, such as TNF. By way of example, it may be possible to induce the expression of—or increase the expression of—TNF by adding bacterial lipopolysaccharide (LPS) and muramyl dipeptide (MDP). In this regard, bacterial LPS and MDP in combination can stimulate TNF production from murine spleen cells in vitro and tumour regression in vivo (Fuks et al Biull Eksp Biol Med 1987 104: 497-499).

In the method of treatment, the subject is preferably a mammal, more preferably a human. For some applications, preferably the human is a woman.

The present invention also covers novel intermediates that are useful to prepare the compounds of the present invention. For example, the present invention covers novel alcohol precursors for the compounds. By way of further example, the present invention covers bis protected precursors for the compounds. Examples of each of these precursors are presented herein. The present invention also encompasses a process comprising each or both of those precursors for the synthesis of the compounds of the present invention.

Steroid Sulphatase

Steroid sulphatase—which is sometimes referred to as steroid sulphatase or steryl sulphatase or “STS” for short—hydrolyses several sulphated steroids, such as oestrone sulphate, dehydroepiandrosterone sulphate and cholesterol sulphate. STS has been allocated the enzyme number EC 3.1.6.2.

STS has been cloned and expressed. For example see Stein et al (J. Biol. Chem. 264:13865-13872 (1989)) and Yen et al (Cell 49:443-454 (1987)).

STS is an enzyme that has been implicated in a number of disease conditions.

By way of example, workers have found that a total deficiency in STS produces ichthyosis. According to some workers, STS deficiency is fairly prevalent in Japan. The same workers (Sakura et al, J Inherit Metab Dis 1997 November; 20(6):807-10) have also reported that allergic diseases—such as bronchial asthma, allergic rhinitis, or atopic dermatitis—may be associated with a steroid sulphatase deficiency.

In addition to disease states being brought on through a total lack of STS activity, an increased level of STS activity may also bring about disease conditions. By way of example, and as indicated above, there is strong evidence to support a role of STS in breast cancer growth and metastasis.

STS has also been implicated in other disease conditions. By way of example, Le Roy et al (Behav Genet 1999 March; 29(2):131-6) have determined that there may be a genetic correlation between steroid sulphatase concentration and initiation of attack behaviour in mice. The authors conclude that sulphatation of steroids may be the prime mover of a complex network, including genes shown to be implicated in aggression by mutagenesis.

STS Inhibition

It is believed that some disease conditions associated with STS activity are due to conversion of a nonactive, sulphated oestrone to an active, nonsulphated oestrone. In disease conditions associated with STS activity, it would be desirable to inhibit STS activity.

Here, the term “inhibit” includes reduce and/or eliminate and/or mask and/or prevent the detrimental action of STS.

STS Inhibitor

In accordance with the present invention, the compound of the present invention is capable of acting as an STS inhibitor.

Here, the term “inhibitor” as used herein with respect to the compound of the present invention means a compound that can inhibit STS activity—such as reduce and/or eliminate and/or mask and/or prevent the detrimental action of STS. The STS inhibitor may act as an antagonist.

The ability of compounds to inhibit oestrone sulphatase activity can be assessed using either intact JEG3 choriocarcinoma cells or placental microsomes. In addition, an animal model may be used. Details on suitable Assay Protocols are presented in following sections. It is to be noted that other assays could be used to determine STS activity and thus STS inhibition. For example, reference may also be made to the teachings of WO-A-99/50453.

In one aspect, for some applications, the compound is further characterised by the feature that if the sulphamate group were to be substituted by a sulphate group to form a sulphate derivative, then the sulphate derivative would be hydrolysable by an enzyme having steroid sulphatase (E.C. 3.1.6.2) activity—i.e. when incubated with steroid sulphatase EC 3.1.6.2 at pH 7.4 and 37° C.

In one preferred embodiment, if the sulphamate group of the compound were to be replaced with a sulphate group to form a sulphate compound then that sulphate compound would be hydrolysable by an enzyme having steroid sulphatase (E.C. 3.1.6.2) activity and would yield a Km value of less than 200 mmolar, preferably less than 150 mmolar, preferably less than 100 mmolar, preferably less than 75 mmolar, preferably less than 50 mmolar, when incubated with steroid sulphatase EC 3.1.6.2 at pH 7.4 and 37° C.

In a preferred embodiment, the compound of the present invention is not hydrolysable by an enzyme having steroid sulphatase (E.C. 3.1.6.2) activity.

For some applications, preferably the compound of the present invention has at least about a 100 fold selectivity to a desired target (e.g. STS and/or aromatase), preferably at least about a 150 fold selectivity to the desired target, preferably at least about a 200 fold selectivity to the desired target, preferably at least about a 250 fold selectivity to the desired target, preferably at least about a 300 fold selectivity to the desired target, preferably at least about a 350 fold selectivity to the desired target.

It is to be noted that the compound of the present invention may have other beneficial properties in addition to or in the alternative to its ability to inhibit STS and/or aromatase activity.

### Assay for Determining STS Activity Using Cancer Cells (Protocol 1)

Inhibition of Steroid Sulphatase Activity in JEG3 cells

Steroid sulphatase activity is measured in vitro using intact JEG3 choriocarcinoma cells. This cell line may be used to study the control of human breast cancer cell growth. It possesses significant steroid sulphatase activity (Boivin et al., J. Med. Chem., 2000, 43: 4465-4478) and is available in from the American Type Culture Collection (ATCC).

Cells are maintained in Minimal Essential Medium (MEM) (Flow Laboratories, Irvine, Scotland) containing 20 mM HEPES, 5% foetal bovine serum, 2 mM glutamine, non-essential amino acids and 0.075% sodium bicarbonate. Up to 30 replicate 25 cm2 tissue culture flasks are seeded with approximately 1×10⁵ cells/flask using the above medium. Cells are grown to 80% confluency and the medium is changed every third day.

Intact monolayers of JEG3 cells in triplicate 25 cm² tissue culture flasks are washed with Earle's Balanced Salt Solution (EBSS from ICN Flow, High Wycombe, U.K.) and incubated for 3-4 hours at 37° C. with 5 μmol (7×10⁵ dpm) [6,7-3H]oestrone-3-sulphate (specific activity 60 Ci/mmol from New England Nuclear, Boston, Mass., U.S.A.) in serum-free MEM (2.5 ml) together with oestrone-3-sulphamate (11 concentrations: 0; 1 fM; 0.01 pM; 0.1 pM; 1 pM; 0.01 nM; 0.1 nM; 1 nM; 0.01 mM; 0.1 mM; 1 mM). After incubation each flask is cooled and the medium (1 ml) is pipetted into separate tubes containing [14C]oestrone (7×103 dpm) (specific activity 97 Ci/mmol from Amersham International Radiochemical Centre, Amersham, U.K.). The mixture is shaken thoroughly for 30 seconds with toluene (5 ml). Experiments have shown that >90% [14C] oestrone and <0.1% [3H]oestrone-3-sulphate is removed from the aqueous phase by this treatment. A portion (2 ml) of the organic phase is removed, evaporated and the 3H and 14C content of the residue determined by scintillation spectrometry. The mass of oestrone-3-sulphate hydrolysed was calculated from the 3H counts obtained (corrected for the volumes of the medium and organic phase used, and for recovery of [14C] oestrone added) and the specific activity of the substrate. Each batch of experiments includes incubations of microsomes prepared from a sulphatase-positive human placenta (positive control) and flasks without cells (to assess apparent non-enzymatic hydrolysis of the substrate). The number of cell nuclei per flask is determined using a Coulter Counter after treating the cell monolayers with Zaponin. One flask in each batch is used to assess cell membrane status and viability using the Trypan Blue exclusion method (Phillips, H. J. (1973) In: Tissue culture and applications, [eds: Kruse, D. F. & Patterson, M. K.]; pp. 406-408; Academic Press, New York).

Results for steroid sulphatase activity are expressed as the mean±1 S.D. of the total product (oestrone+oestradiol) formed during the incubation period (3-4 hours) calculated for 106 cells and, for values showing statistical significance, as a percentage reduction (inhibition) over incubations containing no oestrone-3-sulphamate. Unpaired Student's t-test was used to test the statistical significance of results.

### Assay for Determining STS Activity Using Placental Microsomes (Protocol 2)

Inhibition of Steroid Sulphatase Activity in Placental Microsomes

Sulphatase-positive human placenta from normal term pregnancies are thoroughly minced with scissors and washed once with cold phosphate buffer (pH 7.4, 50 mM) then re-suspended in cold phosphate buffer (5 ml/g tissue). Homogenisation is accomplished with an Ultra-Turrax homogeniser, using three 10 second bursts separated by 2 minute cooling periods in ice. Nuclei and cell debris are removed by centrifuging (4° C.) at 2000 g for 30 minutes and portions (2 ml) of the supernatant are stored at 20° C. The protein concentration of the supernatants is determined by the method of Bradford (Anal. Biochem., 72, 248-254 (1976)).

Incubations (1 ml) are carried out using a protein concentration of 100 mg/ml, substrate concentration of 20 mM [6,7-3H]oestrone-3-sulphate (specific activity 60 Ci/mmol from New England Nuclear, Boston, Mass., U.S.A.) and an incubation time of 20 minutes at 37° C. If necessary eight concentrations of compounds are employed: 0 (i.e. control); 0.05 mM; 0.1 mM; 0.2 mM; 0.4 mM; 0.6 mM; 0.8 mM; 1.0 mM. After incubation each sample is cooled and the medium (1 ml) was pipetted into separate tubes containing [14C]oestrone (7×103 dpm) (specific activity 97 Ci/mmol from Amersham International Radiochemical Centre, Amersham, U.K.). The mixture is shaken thoroughly for 30 seconds with toluene (5 ml). Experiments have shown that >90% [14C]oestrone and <0.1% [3H]oestrone-3-sulphate is removed from the aqueous phase by this treatment. A portion (2 ml) of the organic phase was removed, evaporated and the 3H and 14C content of the residue determined by scintillation spectrometry. The mass of oestrone-3-sulphate hydrolysed is calculated from the 3H counts obtained (corrected for the volumes of the medium and organic phase used, and for recovery of [14C]oestrone added) and the specific activity of the substrate.

### Animal Assay Model for Determining STS Activity (Protocol 3)

Inhibition of Oestrone Sulphatase Activity In Vivo

The compounds of the present invention may be studied using an animal model, in particular in ovariectomised rats. In this model compounds which are oestrogenic stimulate uterine growth.

The compound (0.1 mg/Kg/day for five days) is administered orally to rats with another group of animals receiving vehicle only (propylene glycol). At the end of the study samples of liver tissue were obtained and oestrone sulphatase activity assayed using 3H oestrone sulphate as the substrate as previously described (see PCT/GB95/02638).

### Animal Assay Model for Determining Oestrogenic Activity (Protocol 4)

The compounds of the present invention may be studied using an animal model, in particular in ovariectomised rats. In this model, compounds which are oestrogenic stimulate uterine growth.

The compound (0.1 mg/Kg/day for five days) was administered orally to rats with another group of animals receiving vehicle only (propylene glycol). At the end of the study uteri were obtained and weighed with the results being expressed as uterine weight/whole body weight×100.

Compounds having no significant effect on uterine growth are not oestrogenic.

### Biotechnological Assays for Determining STS Activity (Protocol 5)

The ability of compounds to inhibit oestrone sulphatase activity can also be assessed using amino acid sequences or nucleotide sequences encoding STS, or active fragments, derivatives, homologues or variants thereof in, for example, high-through put screens. Such assays and methods for their practice are taught in WO 03/045925 which is incorporated herein by reference.

In one preferred aspect, the present invention relates to a method of identifying agents that selectively modulate STS, which compounds have the formula (I).

### Assay for Determining Aromatase Activity Using JEG3 Cells (Protocol 6)

Aromatase activity is measured in JEG3 choriocarcinoma cells, obtained from the ATCC. This cell line possesses significant aromatase activity and is widely used to study the control of human aromatase activity (Bhatnager et al., J. Steroid Biochem. Molec. Biol. 2001, 76: 199-202). Cells are maintained in Minimal Essential Medium (MEM, Flow Laboratories, Irvine, Scotland) containing 20 mM HEPES, 10% foetal bovine serum, 2 mM glutamine, non-essential amino acids and 0.075% sodium bicarbonate. Intact monolayers of JEG3 cells (2.5×10⁶ cells) in triplicate 25 cm² tissue culture flasks are washed with Earle's Balanced salt solution (EBSS, from ICN Flow, High Wycombe, UK) and incubated with [1β-³H] androstenedione (2-5 nM, 26 Ci/mmol, New England Nuclear, Boston, Mass., USA) for 30 min with inhibitors over the range of 10 pm-10 μM. During the aromatase reaction, ³H₂O is liberated which can he quantified using a liquid scintillation spectrometer (Beckman-Coulter, High Wycombe, Bucks. UK). This ³H₂O-release method has been widely used to measure aromatase activity (Newton et al., J. Steroid Biochem. 1986, 24: 1033-1039). The number of cell nuclei per flask is determined using a Coulter Counter after treating the cell monolayers with Zaponin.

Results for aromatase activity are expressed as the mean±1 S.D. of the product formed during the incubation period (30 min) calculated for 10⁶ cells and, for values showing a statistical significance, as a percentage reduction (inhibition) over incubations containing no aromatase inhibitor. Unpaired Student's t test was used to test the statistical significance of results. IC₅₀ values were calculated as the concentration of inhibitor required to obtain a 50% inhibition of aromatase activity.

### Animal Assays for Determining Aromatase Activity (Protocol 7)

(i) Inhibition of PMSG-Induced Oestrogen Synthesis

The ability of compounds to inhibit aromatase activity in vivo was tested using a pregnant mare serum gonadotrophin (PMSG)-induced oestrogen synthesis assay. For this, female rats (250 g) were injected with PMSG (200 IU, s.c.). After 72 h rats were administered vehicle (propylene glycol) or various doses of test compounds orally. At 2 h after dosing blood samples were obtained by cardiac puncture (under anaesthesia). Plasma oestradiol levels were measured in control groups and groups receiving drugs. The efficacy of aromatase inhibition was determined by measurement of plasma oestradiol concentrations by radioimmunoassay. This method has been widely used to determine the effectiveness of aromatase inhibitors in vivo (Wouters et al., J. Steroid Biochem., 1989, 32: 781-788).

(ii) Inhibition of Androstenedione Stimulated Uterine Growth in Ovariectomised Rats

Female rats (250 g) were ovariectomised and used to determine the effectiveness of aromatase inhibition on androstenedione stimulated uterine growth. Administration of androstenedione (30 mg/kg/d) for a 2-week period results in a significant increase in uterine growth in ovariectomised animals. This increase in uterine growth is stimulated by oestrogen which is derived from the administered androstenedione as a result of the action of the aromatase enzyme. By co-administration of compounds with androstenedione the extent of aromatase inhibition can be determined by measurements of uterine weights in treated and untreated animals.

Therapy

The compounds of the present invention may be used as therapeutic agents—i.e. in therapy applications.

The term “therapy” includes curative effects, alleviation effects, and prophylactic effects.

The therapy may be on humans or animals, preferably female animals.

Pharmaceutical Compositions

In one aspect, the present invention provides a pharmaceutical composition, which comprises a compound according to the present invention and optionally a pharmaceutically acceptable carrier, diluent or excipient (including combinations thereof.

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Combination Pharmaceutical

The compound of the present invention may be used in combination with one or more other active agents, such as one or more other pharmaceutically active agents.

By way of example, the compounds of the present invention may be used in combination with other STS inhibitors and/or other inhibitors such as an aromatase inhibitor (such as for example, 4-hydroxyandrostenedione (4-OHA)) and/or steroids—such as the naturally occurring neurosteroids dehydroepiandrosterone sulfate (DHEAS) and pregnenolone sulfate (PS) and/or other structurally similar organic compounds. Examples of other STS inhibitors may be found in the above references. By way of example, STS inhibitors for use in the present invention include EMATE, and either or both of the 2-ethyl and 2-methoxy 17-deoxy compounds that are analogous to compound 5 presented herein.

In addition, or in the alternative, the compound of the present invention may be used in combination with a biological response modifier.

The term biological response modifier (“BRM”) includes cytokines, immune modulators, growth factors, haematopoiesis regulating factors, colony stimulating factors, chemotactic, haemolytic and thrombolytic factors, cell surface receptors, ligands, leukocyte adhesion molecules, monoclonal antibodies, preventative and therapeutic vaccines, hormones, extracellular matrix components, fibronectin, etc. For some applications, preferably, the biological response modifier is a cytokine. Examples of cytokines include: interleukins (IL)—such as IL-1, IL-2, IL-3, IL-4, IL-5, IL-6, IL-7, IL-8, IL-9, IL-10, IL-11, IL-12, IL-19; Tumour Necrosis Factor (TNF)— such as TNF-α; Interferon alpha, beta and gamma; TGF-β. For some applications, preferably the cytokine is tumour necrosis factor (TNF). For some applications, the TNF may be any type of TNF—such as TNF-α, TNF-β, including derivatives or mixtures thereof. More preferably the cytokine is TNF-α. Teachings on TNF may be found in the art—such as WO-A-98/08870 and WO-A-98/13348.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The compositions of the present invention may be administered by direct injection. The composition may be formulated for parenteral, mucosal, intramuscular, intravenous, subcutaneous, intraocular or transdermal administration. Depending upon the need, the agent may be administered at a dose of from 0.01 to 30 mg/kg body weight, such as from 0.1 to 10 mg/kg, more preferably from 0.1 to 1 mg/kg body weight.

By way of further example, the agents of the present invention may be administered in accordance with a regimen of 1 to 4 times per day, preferably once or twice per day. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Aside from the typical modes of delivery—indicated above—the term “administered” also includes delivery by techniques such as lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution; a parenteral route where delivery is by an injectable form, such as, for example, an intravenous, intramuscular or subcutaneous route.

Thus, for pharmaceutical administration, the STS inhibitors of the present invention can be formulated in any suitable manner utilising conventional pharmaceutical formulating techniques and pharmaceutical carriers, adjuvants, excipients, diluents etc. and usually for parenteral administration. Approximate effective dose rates may be in the range from 1 to 1000 mg/day, such as from 10 to 900 mg/day or even from 100 to 800 mg/day depending on the individual activities of the compounds in question and for a patient of average (70 Kg) bodyweight. More usual dosage rates for the preferred and more active compounds will be in the range 200 to 800 mg/day, more preferably, 200 to 500 mg/day, most preferably from 200 to 250 mg/day. They may be given in single dose regimes, split dose regimes and/or in multiple dose regimes lasting over several days. For oral administration they may be formulated in tablets, capsules, solution or suspension containing from 100 to 500 mg of compound per unit dose. Alternatively and preferably the compounds will be formulated for parenteral administration in a suitable parenterally administrable carrier and providing single daily dosage rates in the range 200 to 800 mg, preferably 200 to 500, more preferably 200 to 250 mg. Such effective daily doses will, however, vary depending on inherent activity of the active ingredient and on the bodyweight of the patient, such variations being within the skill and judgement of the physician.

Cell Cycling

The compounds of the present invention may be useful in the method of treatment of a cell cycling disorder.

As discussed in “Molecular Cell Biology” 3rd Ed. Lodish et al. pages 177-181 different eukaryotic cells can grow and divide at quite different rates. Yeast cells, for example, can divide every 120 min., and the first divisions of fertilised eggs in the embryonic cells of sea urchins and insects take only 1530 min. because one large pre-existing cell is subdivided. However, most growing plant and animal cells take 10-20 hours to double in number, and some duplicate at a much slower rate. Many cells in adults, such as nerve cells and striated muscle cells, do not divide at all; others, like the fibroblasts that assist in healing wounds, grow on demand but are otherwise quiescent.

Still, every eukaryotic cell that divides must be ready to donate equal genetic material to two daughter cells. DNA synthesis in eukaryotes does not occur throughout the cell division cycle but is restricted to a part of it before cell division.

The relationship between eukaryotic DNA synthesis and cell division has been thoroughly analysed in cultures of mammalian cells that were all capable of growth and division. In contrast to bacteria, it was found, eukaryotic cells spend only a part of their time in DNA synthesis, and it is completed hours before cell division (mitosis). Thus a gap of time occurs after DNA synthesis and before cell division; another gap was found to occur after division and before the next round of DNA synthesis. This analysis led to the conclusion that the eukaryotic cell cycle consists of an M (mitotic) phase, a G₁ phase (the first gap), the S (DNA synthesis) phase, a G₂ phase (the second gap), and back to M. The phases between mitoses (G₁, S, and G₂) are known collectively as the interphase.

Many nondividing cells in tissues (for example, all quiescent fibroblasts) suspend the cycle after mitosis and just prior to DNA synthesis; such “resting” cells are said to have exited from the cell cycle and to be in the G₀ state.

It is possible to identify cells when they are in one of the three interphase stages of the cell cycle, by using a fluorescence-activated cell sorter (FACS) to measure their relative DNA content: a cell that is in G₁ (before DNA synthesis) has a defined amount x of DNA; during S (DNA replication), it has between x and 2x; and when in G₂ (or M), it has 2x of DNA.

The stages of mitosis and cytokinesis in an animal cell are as follows

(a) Interphase. The G₂ stage of interphase immediately precedes the beginning of mitosis. Chromosomal DNA has been replicated and bound to protein during the S phase, but chromosomes are not yet seen as distinct structures. The nucleolus is the only nuclear substructure that is visible under light microscope. In a diploid cell before DNA replication there are two morphologic chromosomes of each type, and the cell is said to be 2n. In G₂, after DNA replication, the cell is 4n. There are four copies of each chromosomal DNA. Since the sister chromosomes have not yet separated from each other, they are called sister chromatids.

b) Early prophase. Centrioles, each with a newly formed daughter centriole, begin moving toward opposite poles of the cell; the chromosomes can be seen as long threads. The nuclear membrane begins to disaggregate into small vesicles.

(c) Middle and late prophase. Chromosome condensation is completed; each visible chromosome structure is composed of two chromatids held together at their centromeres. Each chromatid contains one of the two newly replicated daughter DNA molecules. The microtubular spindle begins to radiate from the regions just adjacent to the centrioles, which are moving closer to their poles. Some spindle fibres reach from pole to pole; most go to chromatids and attach at kinetochores.

(d) Metaphase. The chromosomes move toward the equator of the cell, where they become aligned in the equatorial plane. The sister chromatids have not yet separated.

(e) Anaphase. The two sister chromatids separate into independent chromosomes. Each contains a centromere that is linked by a spindle fibre to one pole, to which it moves. Thus one copy of each chromosome is donated to each daughter cell. Simultaneously, the cell elongates, as do the pole-to-pole spindles. Cytokinesis begins as the cleavage furrow starts to form.

(f) Telophase. New membranes form around the daughter nuclei; the chromosomes uncoil and become less distinct, the nucleolus becomes visible again, and the nuclear membrane forms around each daughter nucleus. Cytokinesis is nearly complete, and the spindle disappears as the microtubules and other fibres depolymerise. Throughout mitosis the “daughter” centriole at each pole grows until it is full-length. At telophase the duplication of each of the original centrioles is completed, and new daughter centrioles will be generated during the next interphase.

(g) Interphase. Upon the completion of cytokinesis, the cell enters the G₁ phase of the cell cycle and proceeds again around the cycle.

It will be appreciated that cell cycling is an extremely important cell process. Deviations from normal cell cycling can result in a number of medical disorders. Increased and/or unrestricted cell cycling may result in cancer. Reduced cell cycling may result in degenerative conditions. Use of the compound of the present invention may provide a means to treat such disorders and conditions.

Thus, the compound of the present invention may be suitable for use in the treatment of cell cycling disorders such as cancers, including hormone dependent and hormone independent cancers.

In addition, the compound of the present invention may be suitable for the treatment of cancers such as breast cancer, ovarian cancer, endometrial cancer, sarcomas, melanomas, prostate cancer, pancreatic cancer etc. and other solid tumours.

For some applications, cell cycling is inhibited and/or prevented and/or arrested, preferably wherein cell cycling is prevented and/or arrested. In one aspect cell cycling may be inhibited and/or prevented and/or arrested in the G₂/M phase. In one aspect cell cycling may be irreversibly prevented and/or inhibited and/or arrested, preferably wherein cell cycling is irreversibly prevented and/or arrested.

By the term “irreversibly prevented and/or inhibited and/or arrested” it is meant after application of a compound of the present invention, on removal of the compound the effects of the compound, namely prevention and/or inhibition and/or arrest of cell cycling, are still observable. More particularly by the term “irreversibly prevented and/or inhibited and/or arrested” it is meant that when assayed in accordance with the cell cycling assay protocol presented herein, cells treated with a compound of interest show less growth after Stage 2 of the protocol I than control cells. Details on this protocol are presented below.

Thus, the present invention provides compounds which: cause inhibition of growth of oestrogen receptor positive (ER+) and ER negative (ER−) breast cancer cells in vitro by preventing and/or inhibiting and/or arresting cell cycling; and/or cause regression of nitroso-methyl urea (NMU)-induced mammary tumours in intact animals (i.e. not ovariectomised), and/or prevent and/or inhibit and/or arrest cell cycling in cancer cells; and/or act in vivo by preventing and/or inhibiting and/or arresting cell cycling and/or act as a cell cycling agonist.

### Cell Cycling Assay (Protocol 7)

Procedure

Stage 1

MCF-7 breast cancer cells are seeded into multi-well culture plates at a density of 105 cells/well. Cells were allowed to attach and grown until about 30% confluent when they are treated as follows:

Control—no treatment

Compound of Interest (COI) 20 μM

Cells are grown for 6 days in growth medium containing the COI with changes of medium/COI every 3 days. At the end of this period cell numbers were counted using a Coulter cell counter.

Stage 2

After treatment of cells for a 6-day period with the COI cells are re-seeded at a density of 10⁴ cells/well. No further treatments are added. Cells are allowed to continue to grow for a further 6 days in the presence of growth medium. At the end of this period cell numbers are again counted.

Cancer

As indicated, the compounds of the present invention may be useful in the treatment of a cell cycling disorder. A particular cell cycling disorder is cancer.

Cancer remains a major cause of mortality in most Western countries. Cancer therapies developed so far have included blocking the action or synthesis of hormones to inhibit the growth of hormone-dependent tumours. However, more aggressive chemotherapy is currently employed for the treatment of hormone-independent tumours.

Hence, the development of a pharmaceutical for anti-cancer treatment of hormone dependent and/or hormone independent tumours, yet lacking some or all of the side-effects associated with chemotherapy, would represent a major therapeutic advance.

It is known that oestrogens undergo a number of hydroxylation and conjugation reactions after their synthesis. Until recently it was thought that such reactions were part of a metabolic process that ultimately rendered oestrogens water soluble and enhanced their elimination from the body. It is now evident that some hydroxy metabolites (e.g. 2-hydroxy and 16alpha-hydroxy) and conjugates (e.g. oestrone sulphate, E1S) are important in determining some of the complex actions that oestrogens have in the body.

Workers have investigated the formation of 2- and 16-hydroxylated oestrogens in relation to conditions that alter the risk of breast cancer. There is now evidence that factors which increase 2-hydroxylase activity are associated with a reduced cancer risk, while those increasing 16alpha-hydroxylation may enhance the risk of breast cancer. Further interest in the biological role of estrogen metabolites has been stimulated by the growing body of evidence that 2-methoxyoestradiol is an endogenous metabolite with anti-mitotic properties. 2-MeOE2 is formed from 2-hydroxy estradiol (2-OHE2) by catechol estrogen methyl transferase, an enzyme that is widely distributed throughout the body.

Workers have shown that in vivo 2-MeOE2 inhibits the growth of tumours arising from the subcutaneous injection of Meth A sarcoma, B16 melanoma or MDA-MB-435 estrogen receptor negative (ER−) breast cancer cells. It also inhibits endothelial cell proliferation and migration, and in vitro angiogenesis. It was suggested that the ability of 2-MeOE2 to inhibit tumour growth in vivo may be due to its ability to inhibit tumour-induced angiogenesis rather than direct inhibition of the proliferation of tumour cells.

The mechanism by which 2-MeOE2 exerts its potent anti-mitogenic and anti-angiogenic effects is still being elucidated. There is evidence that at high concentrations it can inhibit microtubule polymerisation and act as a weak inhibitor of colchicine binding to tubulin. Recently, however, at concentrations that block mitosis, tubulin filaments in cells were not found to be depolymerised but to have an identical morphology to that seen after taxol treatment. It is possible, therefore, that like taxol, a drug that is used for breast and ovarian breast cancer therapy, 2-MeOE2 acts by stabilising microtubule dynamics.

While the identification of 2-MeOE2 as a new therapy for cancer represents an important advance, the bioavailability of orally administered oestrogens is poor. Furthermore, they can undergo extensive metabolism during their first pass through the liver. As part of a research programme to develop a steroid sulphatase inhibitor for breast cancer therapy, oestrone-3-O-sulphamate (EMATE) was identified as a potent active site-directed inhibitor. Unexpectedly, EMATE proved to possess potent oestrogenic properties with its oral uterotrophic activity in rats being a 100-times higher than that of estradiol. Its enhanced oestrogenicity is thought to result from its absorption by red blood cells (rbcs) which protects it from inactivation during its passage through the liver and which act as a reservoir for its slow release for a prolonged period of time. A number of A-ring modified analogues were synthesised and tested, including 2-methoxyoestrone-3-O-sulphamate. While this compound was equipotent with EMATE as a steroid sulphatase inhibitor, it was devoid of oestrogenicity.

We believe that the compound of the present invention provides a means for the treatment of cancers and, especially, breast cancer.

In addition or in the alternative the compound of the present invention may be useful in the blocking the growth of cancers including leukaemias and solid tumours such as breast, endometrium, prostate, ovary and pancreatic tumours.

Therapy Concerning Oestrogen

We believe that some of the compounds of the present invention may be useful in the control of oestrogen levels in the body—in particular in females. Thus, some of the compounds may be useful as providing a means of fertility control—such as an oral contraceptive tablet, pill, solution or lozenge. Alternatively, the compound could be in the form of an implant or as a patch.

Thus, the compounds of the present invention may be useful in treating hormonal conditions associated with oestrogen.

In addition or in the alternative the compound of the present invention may be useful in treating hormonal conditions in addition to those associated with oestrogen. Hence, the compound of the present invention may also be capable of affecting hormonal activity and may also be capable of affecting an immune response.

Neurodegenerative Diseases

We believe that some of the compounds of the present invention may be useful in the treatment of neurodenerative diseases, and similar conditions.

By way of example, it is believed that STS inhibitors may be useful in the enhancing the memory function of patients suffering from illnesses such as amnesia, head injuries, Alzheimer's disease, epileptic dementia, presenile dementia, post traumatic dementia, senile dementia, vascular dementia and post-stroke dementia or individuals otherwise seeking memory enhancement.

TH1

We believe that some of the compounds of the present invention may be useful in TH1 implications.

By way of example, it is believed that the presence of STS inhibitors within the macrophage or other antigen presenting cells may lead to a decreased ability of sensitised T cells to mount a TH1 (high IL-2, IFNγ low IL-4) response. The normal regulatory influence of other steroids such as glucocorticoids would therefore predominate.

Inflamatory Conditions

We believe that some of the compounds of the present invention may be useful in treating inflammatory conditions—such as conditions associated with any one or more of: autoimmunity, including for example, rheumatoid arthritis, type I and II diabetes, systemic lupus erythematosus, multiple sclerosis, myasthenia gravis, thyroiditis, vasculitis, ulcerative colitis and Crohn's disease, skin disorders e.g. psoriasis and contact dermatitis; graft versus host disease; eczema; asthma and organ rejection following transplantation.

By way of example, it is believed that STS inhibitors may prevent the normal physiological effect of DHEA or related steroids on immune and/or inflammatory responses.

The compounds of the present invention may be useful in the manufacture of a medicament for revealing an endogenous glucocorticoid-like effect.

Other Therapies

It is also to be understood that the compound/composition of the present invention may have other important medical implications.

For example, the compound or composition of the present invention may be useful in the treatment of the disorders listed in WO-A-99/52890—viz:

In addition, or in the alternative, the compound or composition of the present invention may be useful in the treatment of the disorders listed in WO-A-98/05635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the compound or composition of the present invention may be useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); antiinflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the composition of the present invention may be useful in the treatment of disorders listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

In addition, or in the alternative, the compound or composition of the present invention may be useful in the treatment of the disorders listed selected from endometriosis, uterus fibromyoma, induction of mono-ovulation (in polycystic ovarian disease [PCOD] patients). induction of multiple follicullar development in (ART patients), preterm labor/cervical incompetency and recurrent abortion.

Compound Preparation

The compounds of the present invention may be prepared by reacting an appropriate alcohol with a suitable chloride. By way of example, the sulphamate compounds of the present invention may be prepared by reacting an appropriate alcohol with a suitable sulfamoyl chloride, of the formula R⁴R⁵NSO₂Cl.

Typical conditions for carrying out the reaction are as follows.

Sodium hydride and a sulfamoyl chloride are added to a stirred solution of the alcohol in anhydrous dimethyl formamide at 0° C. Subsequently, the reaction is allowed to warm to room temperature whereupon stirring is continued for a further 24 hours. The reaction mixture is poured onto a cold saturated solution of sodium bicarbonate and the resulting aqueous phase is extracted with dichloromethane. The combined organic extracts are dried over anhydrous MgSO₄. Filtration followed by solvent evaporation in vacuo and co-evaporated with toluene affords a crude residue which is further purified by flash chromatography.

Preferably, the alcohol is derivatised, as appropriate, prior to reaction with the sulfamoyl chloride. Where necessary, functional groups in the alcohol may be protected in known manner and the protecting group or groups removed at the end of the reaction.

Preferably, the sulphamate compounds are prepared according to the teachings of Page et al (1990 Tetrahedron 46; 2059-2068).

The phosphonate compounds may be prepared by suitably combining the teachings of Page et al (1990 Tetrahedron 46; 2059-2068) and PCT/GB92/01586.

The sulphonate compounds may be prepared by suitably adapting the teachings of Page et al (1990 Tetrahedron 46; 2059-2068) and PCT/GB92/01586.

The thiophosphonate compounds may be prepared by suitably adapting the teachings of Page et al (1990 Tetrahedron 46; 2059-2068) and PCT/GB91/00270.

Preferred preparations are also presented in the following text.

Preferred preparations are also presented in the following text.

### SUMMARY

In summation, the present invention provides novel compounds for use as steroid sulphatase inhibitors and/or aromatase inhibitors and/or modulators of apoptosis and/or modulators of cell cycling and/or cell growth, and pharmaceutical compositions containing them.

### EXAMPLES

The present invention will now be described in further detail by way of example only with reference to the accompanying figures in which:—

FIG. 1 shows.

The present invention will now be described only by way of example. However, it is to be understood that the examples also present preferred compounds of the present invention, as well as preferred routes for making same and useful intermediates in the preparation of same.

Syntheses

Synthetic Routes

Compounds in accordance with the present invention were synthesised in accordance with the synthetic routes and schemes.

The present invention will now be described only by way of example. However, it is to be understood that the examples also present preferred compounds of the present invention, as well as preferred routes for making same and useful intermediates in the preparation of same.

### 2-Ethyl-3-O-tert-butyl-dimethyl-silyl-17-methanesulfonylmethyl estrone 6

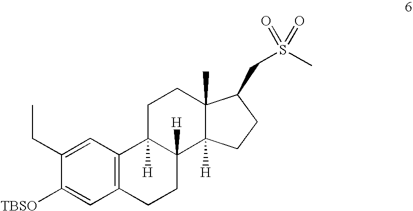

A room temperature solution of 2-ethyl-3-O-TBS estrone-17-methylsulfanylmethyl estrone 4 (500 mg) in dichloromethane (25 mL) was treated with m-CPBA (764 mg, 4 mmol). The reaction was stirred for 16 h then washed with aqueous sodium hydroxide (40 mL, 1M), water (40 mL) and brine (40 mL), dried and evaporated. The crude product, a yellow oil, was purified by column chromatography (4:1 to 3:1 hexane/ethyl acetate) to give the desired sulphone 6 (170 mg). The product, a colourless oil, showed δ_{H} 7.03 (1H, s, ArH), 6.47 (1H, s, ArH), 3.10-3.20 (1H, m, CH_{A}H_{B}SO₂), 2.92 (3H, s, SO₂Me), 2.74-2.94 (3H, m, 6-CH₂ and CH_{B}H_{A}SO₂), 2.55 (214, q, J 7.4, CH₂Me), 1.20-2.40 (14H, m), 1.15 (3H, t, J 7.4, CH₂Me), 0.99 (9H, s, t-Bu), 0.64 (3H, s, 18-CH₃), and 0.21 (6H, S, SiMe₂);

### 2-Ethyl-17-methanesulfonylmethyl estrone 7

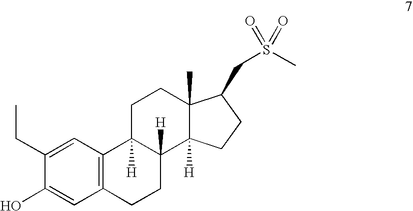

A solution of the 2-ethyl-3-O-tert-butyl-dimethyl-silyl-17-methanesulfonylmethyl estrone 6 (135 mg, 0.29 mmol) in THF (5 mL) was treated with a solution of tetra-butyl ammonium fluoride in THF (0.5 mL. 0.5 mmol) and maintained at ambient temperature for 16 h. The reaction was then diluted with ethyl acetate (25 mL), washed with water (20 mL) and brine (25 mL), then dried and evaporated. The product was crystallised from ether/hexane to give the desired sulfone 7 as a white solid mp ° C. (85 mg over 3 crops, 77%) which showed δ_{H} 7.02 (1H, s, ArH), 6.48 (1H, s, ArH), 4.60 (1H, s, OH), 3.13 (1H, dd, J 13.3 and 2.3, CH_{A}H_{B}SO₂), 2.93 (3H, s, SO₂Me), 2.85-2.92 (1H, m, CH_{B}H_{A}SO₂), 2.76-2.84 (2H, m, 6-CH₂), 2.58 (2H, q, J 7.4, CH₂Me), 1.26-2.38 (14H, m), 1.21 (3H, t, J 7.4, CH₂Me) and 0.65 (3H, s, 18-CH₃); δ_{C} 151.0, 135.2, 132.2, 127.0, 126.1, 115.1, 56.8, 53.8, 44.2, 44.0, 43.5, 41.8, 38.9, 37.1, 29.3, 28.7, 27.8, 26.4, 24.7, 23.1, 14.6 and 12.9; m/z [ES−] 1375.3 (M⁺−H, 100%); HRMS [FAB+] 376.20722, C₂₂H₃₂SO₃ requires 376.20721. UV λₘₐₓ 282 nm.

### 2-Ethyl-3-O-sulfamoyl-17-methanesulfonylmethyl estrone 8

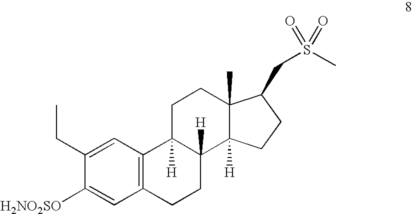

Sulfamoyl chloride (150 mg, 1.3 mmol) was cooled to 0° C., dissolved in dimethyl acetamide (2 mL) and then after 5 minutes treated with 2-ethyl-17-methanesulfanyl-methyl estrone 7 (60 mg, 0.16 mmol). External cooling was removed after 15 minutes and the reaction was left to stir at ambient temperature for 3 h. The reaction was then diluted in ethyl acetate (15 mL), poured onto brine (15 mL) and the organic layer was separated. The organic extract washed with water (3×10 mL), brine (10 mL), dried and evaporated to give a yellow powder. Crystallisation from ethyl acetate/hexane afforded the desired product 8 as white crystals (42 mg, 58%) which showed δ_{H} (CDCl₃) 7.17 (1H, s, ArH), 7.07 (1H, s, ArH), 4.95 (2H, s, NH₂), 3.10-3.18 (1H, m, CH_{A}H_{B}SO₂), 2.76-2.95 (6H, m, SO₂Me, CH_{B}H_{A}SO₂ and 6-CH₂ including 2.92 (3H, s, SO₂Me)), 2.66 (2H, q, J 7.4, CH₂Me), 1.16-2.40 (17H, m including 1.20 (3H, t, J 7.4, CH₂Me) and 0.64 (3H, s, 18-CH₃); δ_{C} (CDCl₃+CD₃OD) 146.1, 138.6, 135.4, 133.6, 126.5, 121.4, 56.6, 53.8, 44.1, 44.0, 43.3, 41.6, 38.4, 36.9, 29.1, 28.6, 27.5, 26.1, 24.6, 23.0, 14.6 and 12.9. m/z [APCI−] 454.29 (M⁺−H, 100%).

### 2-Methoxy-3-O-tert-butyldimethylsilyl-17-(methylsulfanylmethyl)-estra-1,3,5-triene

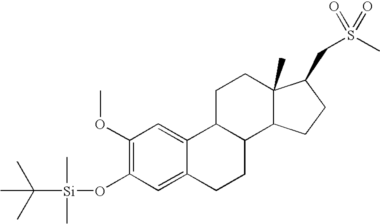

A solution of 2-methoxy-3-O-tert-butyldimethylsilyl-17-(methylthiomethyl)-estra-1,3,5-triene (220 mg, 0.48 mmol) in chloroform (10 mL) was treated with mCPBA (300 mg, 1.3 mmol) and then stirred for 1 h at rt. The reaction was then washed with sodium bicarbonate solution, then water, then brine, dried and evaporated. The resultant oil was purified by column chromatography (0 to 6% acetone in chloroform) to give the desired sulphone, a colourless oil, as a mixture of diastereoismers at C-17 (120 mg, %) which showed δ_{H} 0.14 (6H, 2×s, SiMe₂), 0.66 (1.7H, s, s, 18-CH₃ major isomer), 0.91(1.3H, s, 18-CH₃ minor isomer), 0.98 (9H, s, t-Bu), 1.20-2.36 (14H, m), 2.70-2.80 (2H, m, 6-CH₂), 2.84-2.96 (4H, m including 2.93 (3H, s, MeSO₂)), 3.10-3.17 (1H, m, CHₐH_{b}SO₂), 3.76 (3H, s), 6.56 (1H, s) and 6.75 (1H, s). C₂₇H₄₄O₄SSi.

### 2-Methoxy-3-hydroxy-17β-(methylsulfanylmethyl)-estra-1,3,5-triene

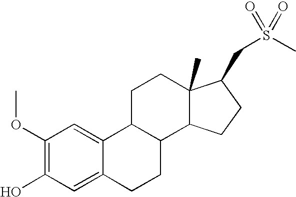

To a solution of 2-methoxy-3-O-tert-butyldimethylsilyl-17-(methylsulfanylmethyl)-estra-1,3,5-triene (120 mg) in THF (1 mL) was added TBAF (0.275 mL of a 1M solution in THF). Complete conversion of starting material was observed after two minutes at which time the reaction was diluted in ethyl acetate and then washed with water and brine, then dried and evaporated. Column chromatography (0 to 10% acetone in chloroform) afforded the desired product as a single diastereoisomer (58 mg) as a white crystalline solid.

Recrystallisation from acetone/hexane gave white needles m.p. 193-94° C. which showed δ_{H} 0.66 (3H, s, 18-CH₃), 1.25-2.36 (14H, m), 2.72-2.82 (2H, m, 6-CH₂), 2.85-2.95 (1H, m, CHₐH_{b}SO₂), 2.93 (3H, s, MeSO₂), 3.12-3.20 (1H, m, CHₐH_{b}SO₂), 3.86 (3H, s, OMe), 5.43 (1H, s, OH), 6.64 (1H, s,) and 6.78 (1H, s).

### 2-Methoxy-3-O-sulfamoyl-17β-(methylsulfanylmethyl)-estra-1,3,5-triene

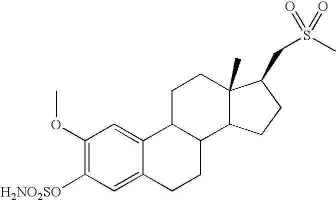

To a 0° C. solution of sulfamoyl chloride (0.5 mmol) in DMA (1.5 mL) was added 2-methoxy-3-hydroxy-17β-(methylsulfanylmethyl)-estra-1,3,5-triene (45 mg). The reaction was allowed to come to room temperature and then stirred for a further 3 h before addition of ethyl acetate (30 mL). The mixtured was then washed with water and brine, dried and evaporated to give the crude sulfamate as a white powder. Column chromatography (0 to is 15% acetone in chloroform) afforded the desired product as a white powder which showed δ_{H} (d₆-acetone) 0.76 (3H, s, 18-CH₃), 1.30-2.46 (H, m), 2.78-2.95 (3H, m, 6-CH2 & CHₐH_{b}SO₂), 2.98 (3H, s, CH₃SO₂), 3.26-3.34 (1H, m, CHₐH_{b}SO₂), 3.87 (3H, s, OMe), 6.95 (2H, s, NH₂), 7.05 (1H, s, ArH) and 7.07 (1H, s, ArH). m/z [APCI−] 456.2 (100%, M—H).

### 2-Ethyl-17-methanesulfinylmethyl estrone 9

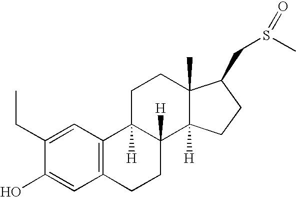

A rt solution of 2-ethyl-3-O-tert-butyl-dimethyl-silyl-17-methylsulfanylmethyl estrone 4 (100 mg, 0.21 mmol) in dichloromethane (5 mL) was treated with mCPBA (160 mg) in four portions until tlc showed no residual starting material remained (2.5 h). The reaction was then treated diluted in dichloromethane (20 mL) and washed with aqueous ammonia (3×20 mL, 2M), water (20 mL) and brine (20 mL) then dried and evaporated to give a colourless oil. Chromatography (5% MeOH in DCM) gave the desired sulfoxide 9 (82 mg) as a colourless oil which shows characteristic resonances at 2.62 and 2.60 (3H (both diastereoisomers), SOMe). Selected data δ_{H} 7.03 (1H, s, ArH), 6.46 (1H, s, ArH), 2.70-2.94 (4H, m, 6-CH₂ and CH₂SO), 2.50-2.62 (5H, m, CH₂Me and SOMe), 1.15 (3H, t, J 7.4, CH₂Me), 0.99 (9H, s, t-Bu), 0.86 (18-CH₃), 0.86 (18-CH₃, minor isomer, d, J 4.9), 0.68 (18-CH₃, major isomer, d, J 3.7) and 0.21 (6H, s, SiMe₂). m/z [APCI−] 475.3 (M⁺+H, 100%). HRMS [FAB+] 474.29878. The silyl ether was dissolved in THF (5 mL) and cleaved by treatment with TBAF (1 mL, 1M in THF) over 2 h to give the desired sulfoxide as a colourless oil which showed δ_{H} 7.02 (1H, s, ArH), 6.51 (1H, s, ArH), 5.65 (1H, s, OH), 2.70-2.94 (4H, m, 6-CH₂ and CH₂SO), 2.50-2.62 (5H, m, CH₂Me and SOMe including 2.60 (d, J=4.0, SOMe major isomer)), 1.15 (3H, t, J 7.4, CH₂Me), 0.86 (18-CH₃), 0.84 (18-CH₃, minor isomer, d, J 5.2) and 0.65 (18-CH₃, major isomer, d, J 4.0); m/z [APCI−] 361.3 (M⁺+H, 100%). HRMS [FAB+] 360.21230.

### 2-Ethyl-3-O-sulfamoyl-17-β-methyl-17-deoxy estrone 10

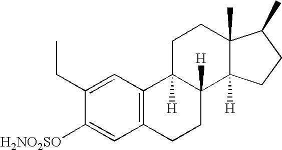

A solution of 2-ethyl-3-O-sulfamoyl 17-methylene estrone (100 mg) in ethanol (10 mL) was hydrogenated in the presence of Pd/C (25 mg, 5%) for 16 h. The reaction was then filtered through celite and evaporated to give a colourless oil which solidified on standing. The product, 2-ethyl-3-O-sulfamoyl-17-β-methyl-17-deoxy estrone 10 (95 mg), showed δ_{H} (CDCl₃) 7.18 (1H, s, ArH), 7.04 (1H, s, ArH), 5.01 (2H, br, NH₂), 2.79-2.86 (2H, m, 6-CH₂), 2.68 (2H, q, J 7.4, CH₂Me), 1.16-2.34 (17H, m including 1.21 (3H, t, J 7.4, MeCH₂)), 0.88 (3H, d, J 7.0, CH₃CH) and 0.58 (3H, s, 18-CH₃); δ_{C} 145.8, 139.8, 136.0, 133.3, 126.9, 121.2, 54.9, 45.2, 44.4, 42.3, 38.7, 37.5, 30.3, 29.4, 27.8, 26.4, 24.5, 23.2, 14.8, 14.0 and 12.1; HRMS [FAB+] 377.20246.

### 2-Ethyl-3-O-sulfamoyl-17-β-ethyl-17-deoxy estrone 11

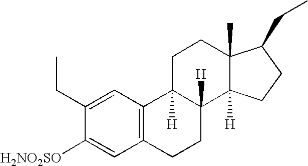

A solution of 2-ethyl-3-O-sulfamoyl 17-ethylidene estrone (80 mg) in ethanol (10 mL) was hydrogenated in the presence of Pd/C (25 mg, 5%) for 16 h. The reaction was then filtered through celite and evaporated to give a white solid (80 mg). The product, 2-ethyl-3-O-sulfamoyl-17-β-ethyl-17-deoxy estrone 11, was crystallized from ethyl acetate/hexane and showed δ_{H} (CDCl₃) 7.18 (1H, s, ArH), 7.04 (1H, s, ArH), 5.01 (2H, br, NH₂), 2.78-2.86 (2H, m, 6-CH₂), 2.68 (2H, q, J 7.4, CH₂Me), 1.05-2.32 (19H, m including 1.21 (3H, t, J 7.4, MeCH₂)), 0.90 (3H, t, J 7.0, CH₃CH₂) and 0.60 (3H, s, 18-CH₃); δ_{C} 145.8, 139.8, 136.0, 133.3, 126.8, 121.2, 55.0, 53.2, 44.5, 42.4, 38.5, 38.0, 29.4, 28.3, 28.0, 26.7, 24.4, 23.3, 23.2, 14.8, 13.5 and 12.6.

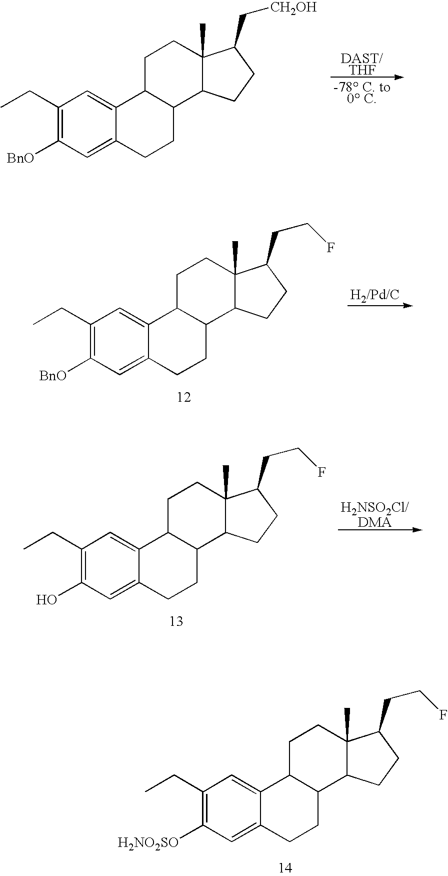

### 3-Benzyloxy-2-ethyl-17β-(2-fluoro-ethyl)-17-deoxy estrone 12

A solution of 3-benzyloxy-2-ethyl-17β-(2-hydroxyethyl)-17-deoxy estrone (0.84 g, 2 mmol) in dry THF (20 ml) under nitrogen was cooled to −78° C. before diethylaminosulfur trifluoride (DAST) (0.40 ml, 3 mmol) was added dropwise. The mixture was stirred at −78° C. for 4 hours then at 0° C. for 42 hours. After addition of saturated aqueous NaHCO₃ (10 ml) the organic layer was extracted with ethyl acetate (100 ml). The organic layer was then washed with water, brine and dried over MgSO₄. The solvents were removed under vacuum and the residual solid was purified by column chromatography (hexane/ethyl acetate 50:1) to give 3-benzyloxy-2-ethyl-17β-(2-fluoro-ethyl)-17-deoxy estrone 12 as a white powder, 0.42 g (50%), mp=114-115° C.; ¹H NMR (CDCl₃, 270 MHz): 0.66 (s, 3H, CH₃), 1.30 (t, J=7.4 Hz, 3H, CH₃), 1.32-1.66 (m, 9H), 1.81-1.87 (m, 1H), 1.92-2.04 (m, 4H), 2.29 (m, 1H), 2.41 (m, 1H), 2.76 (q, J=7.4 Hz, 2H, CH₂), 2.92 (m, 2H, H6), 4.50 (m, 1H, CH₂F), 4.62 (m, 1H, CH₂F), 5.12 (s, 2H, CH₂Ph), 6.72 (s, 1H, ArH), 7.20 (s, 1H, ArH), 7.37-7.54 (m, 5H, Ph).

### 2-Ethyl-17β-(2-fluoro-ethyl)-17-deoxy estrone 13

To a solution of 3-benzyloxy-2-ethyl-17β-(2-fluoro-ethyl)-17-deoxy estrone 12 (0.42 g, 1 mmol) in THF (2 ml) and ethanol (20 ml) was added 30 mg of 5% Pd/C and the mixture was stirred under hydrogen for 24 hours. The suspension was filtered over celite/sand and the solvents evaporated under vacuum. The residual oil was purified by column chromatography (hexane/ethyl acetate 20/1 to 15:1) to give the desired product 13 as a white powder, 0.42 g (50%), mp=138-139° C.; ¹H NMR (CDCl₃, 270 MHz): 0.63 (s, 3H, CH₃), 1.22 (t, J=7.4 Hz, 3H, CH₃), 1.25-1.61 (m, 10H), 1.73-1.98 (m, 4H), 2.14-2.35 (m, 2H), 2.59 (q, J=7.4 Hz, 2H, CH₂), 2.77 (m, 2H, H6), 4.38 (m, 1H, CH₂F), 4.50-4.60 (m, 2H, CH₂F and OH), 6.49 (s, 1H, ArH), 7.05 (s, 1H, ArH). ¹³C NMR (CDCl₃): 12.6 (CH₃), 14.5, 23.1, 24.5, 26.6, 27.9, 28.3, 29.4, 31.2 (d, J=19.2 Hz, CH₂CH₂F), 37.7, 38.9, 42.5, 44.2, 46.7 (d, J=5.4 Hz, CHCH₂CH₂F), 54.6, 84.1 (d, J=164 Hz, CH₂F), 115.3, 126.4, 127.2, 132.9, 135.6, 151.1. LRMS: 330.22 calcd. C₂₂H₃₁OF, 330.24.

### 2-Ethyl-3-O-sulfamoyl-17β-(2-fluoro-ethyl)-17-deoxy estrone 14

A solution of NH₂SO₂Cl (0.6 mmol) in DMA (2 ml) cooled to 0° C. was added to phenol 13 (66 mg, 0.2 mmol) and the mixture was stirred for 24 hours at room temperature under nitrogen. After addition of water (10 ml) the organics were extracted with ethyl acetate (2×50 ml). The organic layer was successively washed with water, brine and dried over MgSO₄. The solvent was removed under vacuum and the residual solid was purified by column chromatography (hexane/ethyl acetate 20/1). Recrystallisation from hexane/ethyl acetate (20/1) gave 14 as a white solid, 53 mg (66%), mp=152-153° C. ¹H NMR (CDCl₃, 270 MHz): 0.63 (s, 3H, CH₃), 1.21 (t, J=7.4 Hz, 3H, CH₃), 1.23-1.59 (m, 10H), 1.73-1.97 (m, 4H), 2.16-2.35 (m, 2H), 2.68 (q, J=7.4 Hz, 2H, CH₂), 2.82 (m, 2H, H6), 4.38 (m, 1H, CH₂F), 4.55 (m, 1H, CH₂F), 4.90 (s, 2H, NH₂), 6.49 (s, 1H, ArH), 7.05 (s, 1H, ArH). ¹³C NMR (CDCl₃, 400 MHz): 12.5 (CH₃), 14.7, 23.1, 24.4, 26.3, 27.6, 28.2, 29.3, 31.2 (d, J=18.4 Hz, CH₂CH₂F), 37.6, 38.5, 42.4, 44.4, 46.7 (d, J=5.4 Hz, CHCH₂CH₂F), 54.6, 83.9 (d, J=164 Hz, CH₂F), 121.4, 127.0, 133.6, 136.1, 139.8, 146.1.

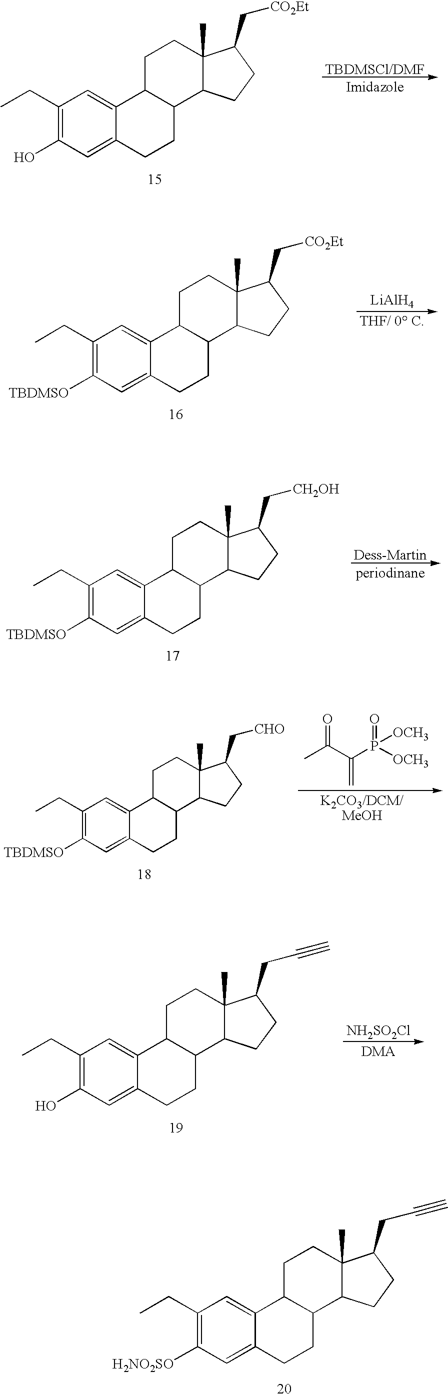

### 2-Ethyl-3-O-TBS-17β-(acetic acid ethyl ester) 17-deoxy estrone 16

A solution of ethyl ester 15 (370.5 mg, 1 mmol), TBDMSCl (160 mg, 1.05 mmol) and imidazole (136 mg, 2 mmol) in 5 ml DMF was stirred at room temperature under nitrogen for 8 hours. After addition of water the organics were extracted with ethyl acetate and the organic layer washed with water, brine, dried over magnesium sulfate and concentrated under reduced pressure. The resulting oil was purified by flash chromatography (hexane/ethyl acetate 50:1) to give 16 as a white powder, 450 mg (93%), mp=94-95° C.; ¹H NMR (CDCl₃, 270 MHz): 0.21 (s, 6H, CH₃), 0.63 (s, 3H, CH₃), 0.99 (s, 9H, (CH₃)₃CSi), 1.15 (t, J=7.4 Hz, 3H, CH₃), 1.26 (t, J=7.3 Hz, 3H, CH₃), 1.28-1.60 (m, 6H), 1.72-2.01 (m, 4H), 2.09-2.43 (m, 3H), 2.55 (q, J=7.3 Hz, 2H, CH₂), 2.76 (m, 2H, H6), 4.12 (q, J=7.4 Hz, 2H, CH₂O), 6.46 (s, 1H, ArH), 7.04 (s, 1H, ArH).

### 2-Ethyl-3-O-TBS-17β-(2-hydroxyethyl) estrone 17

A solution of 16 (390 mg, 0.8 mmol) in 30 ml dry THF stirred under nitrogen was cooled to 0° C. and LiAlH₄ was added portion wise. After 2 hours at 0° C. ice and water were added and the mixture was acidified with NH₄Cl before extraction with ethyl acetate. The organic layer washed with water, brine, dried over magnesium sulfate. The solvent was removed under reduced pressure and the resulting solid purified by flash chromatography (hexane/ethyl acetate 20:1 to 10:1) to give 17 as a white powder, 335 mg (95%), mp=123-124° C.; ¹H NMR (CDCl₃, 270 MHz): 0.21 (s, 6H, CH₃), 0.62 (s, 3H, CH₃), 0.99 (s, 9H, (CH₃)₃CSi), 1.15 (t, J=7.4 Hz, 3H, CH₃), 1.20-1.95 (m, 15H), 2.12-2.32 (m, 2H), 2.55 (q, J=7.4 Hz, 2H, CH₂), 2.76 (m, 2H, H6), 3.58-3.74 (m, 2H, CH₂OH), 6.46 (s, 1H, ArH), 7.04 (s, 1H, ArH).

### 2-Ethyl-3-O-TBS estrone-17β-(2-ethylaldehyde) 18

A solution of 17 (310 mg, 0.7 mmol) in 10 ml DCM stirred under nitrogen was cooled to 0° C. before Dess-Martin periodinane (0.68 g, 1.6 mmol) was added portion wise. The solution was stirred for 6 hours at 0° C. 100 ml diethyl ether and 5 ml of a 1M aqueous of sodium hydroxide solution were added and the mixture stirred for 30 minutes. The organic layer washed with water, brine, dried over magnesium sulfate and the solvents removed under reduced pressure. The resulting oil was purified by flash chromatography (hexane/ethyl acetate 40:1 to 10:1) to afford 18 (35%) as a white powder, 110 mg (35%), mp=78-80° C.; ¹H NMR (CDCl₃, 270 MHz): 0.23 (s, 6H, CH₃), 0.64 (s, 3H, CH₃), 1.00 (s, 9H, (CH₃)₃CSi), 1.16 (t, J=7.3 Hz, 3H, CH₃), 1.23-2.04 (m, 14H), 2.51 (m, 1H, H1′), 2.56 (q, J=7.3 Hz, 2H, CH₂), 2.77 (m, 2H, H6), 6.48 (s, 1H, ArH), 7.05 (s, 1H, ArH), 9.79 (t, J=2.2 Hz, 1H, CHO).

### 2-Ethyl-17β-(prop-2-ynyl) estrone 19

A mixture of (1-Diazo-2-oxo-propyl)-phosphonic acid dimethyl ester (0.19 g, 1.0 mmol) and dry K₂CO₃ in 2.5 ml dry methanol was stirred under nitrogen and cooled to 0° C. before 18 (130 mg, 0.3 mmol) in 1 ml DCM was added drop wise. The mixture was stirred for 24 hours at room temperature, water (10 ml) and DCM (50 ml) added to the solution and the organic layer washed with water and brine successively, dried over MgSO₄. After evaporation of the solvent under reduced pressure, the resulting oil was purified by flash chromatography hexane/ethyl acetate 40:1 to 10:1) to give 19 as a colourless oil, 60 mg (62%). ¹H NMR (CDCl₃, 270 MHz): 0.63 (s, 3H, CH₃), 1.21 (t, J=7.3 Hz, 3H, CH₃), 1.20-1.55 (m, 7H), 1.60-1.76 (m, 2H), 1.80-2.10 (m, 5H), 2.12-2.31 (m, 3H), 2.57 (q, J=7.3 Hz, 2H, CH₂), 2.78 (m, 2H, H6), 4.58 (s, 1H, OH), 6.45 (s, 1H, ArH), 7.04 (s, 1H, ArH). ¹³C NMR (CDCl₃, 100 MHz): 13.0 (CH₃), 14.4, 19.2, 23.1, 24.1, 26.5, 27.8, 28.5, 29.3, 37.9, 38.9, 42.5, 44.1, 49.6, 54.7, 68.2, 84.6, 115.2, 126.3, 127.1, 132.9, 135.6 and 151.1. NB: In addition 7% of the 3-O-TBDMS protected alkyne product was also isolated.

### 2-Ethyl-3-O-sulfamoyl-17β-(prop-2-ynyl) estrone 20

A solution of sulfamoyl chloride (0.1 mmol) in DMA (1 ml) cooled to 0° C. was added to 19 (50 mg, 0.16 mmol) and the mixture was stirred for 24 hours at room temperature under nitrogen. After addition of water (10 ml) the organics were extracted with ethyl acetate (2×50 ml). The organic layer was successively washed with water, brine and dried over MgSO₄. The solvent was removed under vacuum and the residual solid was purified by flash chromatography (hexane/ethyl acetate 10:1 to 7:1) to give 20 as a colourless oil, 40 mg (62%); ¹H NMR (CDCl₃, 270 MHz): 0.61 (s, 3H, CH₃), 1.18 (t, J=7.3 Hz, 3H, CH₃), 1.20-1.74 (m, 10H), 1.82-1.92 (m, 1H), 1.97-2.10 (m, 3H), 2.14-2.30 (m, 3H), 2.66 (q, J=7.3 Hz, 2H, CH₂), 2.79 (m, 2H, H6), 4.97 (br, 2H, NH₂), 7.03 (s, 1H, ArH), 7.15 (s, 1H, ArH).

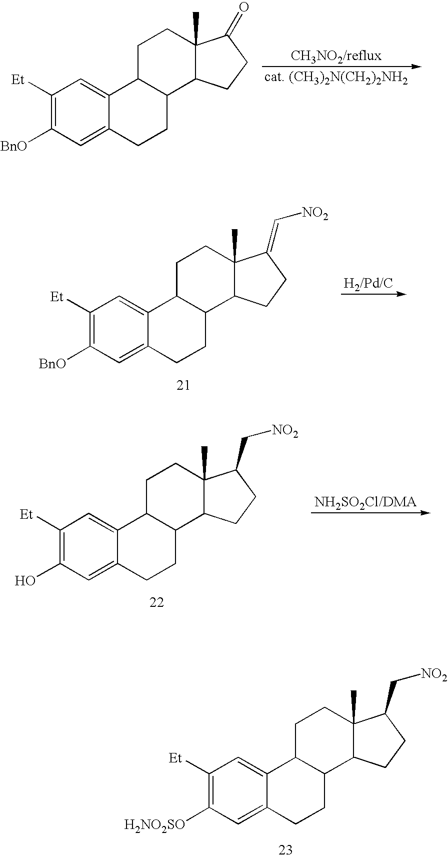

### 3-Benzyloxy-2-ethyl-17-(nitro)methylene estrone 21

A solution of 2-ethyl-3-benzyloxy estrone (5 mmol) in 60 ml toluene was refluxed in a RB flask equipped with a Dean-Stark trap and condenser until ca 20 ml of toluene distilled over, N,N-dimethylethylenediamine (0.1 ml, 0.9 mmol) was then added and the resulting solution refluxed for 24 hours. After cooling to rt the solvent was evaporated under vacuum and the residual solid purified by column chromatography (hexane/ethylacetate) to give 3-benzyloxy-2-ethyl-17-(nitro)methylene estrone 21 as a white powder, 1.6 g (74%), mp=78-79° C.; ¹H NMR (CDCl₃, 270 MHz): 0.96 (s, 3H, CH₃), 1.21 (t, J=7.4 Hz, 3H, CH₃), 1.35-1.62 (m, 6H), 1.99 (m, 3H), 2.27 (m, 1H), 2.47 (m, 1H), 2.66 (q, J=7.4 Hz, 2H, CH₂), 2.85 (m, 2H, H6), 3.09 (m, 2H, H16), 5.04 (s, 2H, CH₂Ph), 6.64 (s, 1H, ArH), 6.92 (dd, J=2.5 and 2.2 Hz, 1H, CHNO₂), 7.10 (s, 1H, ArH), 7.29-7.46 (m, 5H, Ph).

### 2-Ethyl-17β-nitromethyl-17-deoxy estrone 22

To solution of 21 (3 mmol) in 10 ml THF and 60 ml ethanol was added 40 mg of 5% Pd/C. The mixture was stirred at room temperature under hydrogen and the reaction was monitored by TLC. The suspension was then filtered through celite/sand and the solvents evaporated under vacuum. The residual solid was purified by chromatography (hexane/ethylacetate 10/1 to 5/1) and then recrystallized from hexane/ethylacetate (6/1) to give 3-benzyloxy-2-ethyl-17β-nitromethyl-17-deoxy estrone 22 as a white powder, 0.65 g (63%), mp=132-133° C.; ¹H NMR (CDCl₃, 270 MHz): 0.70 (s, 3H, CH₃), 1.21 (t, J=7.4 Hz, 3H, CH₃), 1.30-1.56 (m, 7H), 1.75-2.05 (m, 4H), 2.16-2.35 (m, 3H), 2.58 (q, J=7.4 Hz, 2H, CH₂), 2.79 (m, 2H, H6), 4.25 (dd, J=11.6 and 9.2 Hz, 1H, CH₂NO₂), 4.48 (dd, J=11.6 and 5.9 Hz, 1H, CH₂NO₂), 4.49 (s, H, OH), 6.49 (s, 1H, ArH), 7.02 (s, 1H, ArH). ¹³C NMR (CDCl₃, 400 MHz): 13.1(CH₃), 14.9, 23.5, 24.4, 26.7, 26.9, 28.1, 29.6, 37.6, 39.0, 43.0, 44.2, 49.1, 54.8, 115.4, 126.5, 127.4, 132.5, 135.6, and 151.3; Microanalysis: C, 73.50 (expected 73.44); H, 8.52 (expected 8.51); N, 4.01 (expected 4.08).

### 2-Ethyl-3-O-sulfamoyl-17β-nitromethyl-17-deoxy estrone 23

A solution of NH₂SO₂Cl (3 mmol) in DMA (2 ml) cooled to 0° C. was added to 2-ethyl-17β-nitromethyl-17-deoxy estrone 22 (1 mmol) and the mixture was stirred for 24 hours at room temperature under nitrogen. After addition of water (10 ml) the organics were extracted with ethyl acetate (2×50 ml). The organic layer was successively washed with water, brine and dried over MgSO₄. The solvent was removed under vacuum and the residual solid was purified by column chromatography (hexane/ethyl acetate) followed by recrystallization from hexane/ethyl acetate (6/1) to give 2-ethyl-3-O-sulfamoyl17β-nitromethyl-17-deoxy estrone 23 as a white powder, 0.31 g (74%), mp=203-204° C.; ¹H NMR (CDCl₃, 270 MHz): 0.70 (s, 3H, CH₃), 1.20 (t, J=7.4 Hz, 3H, CH₃), 1.25-1.53 (m, 7H), 1.78-2.05 (m, 4H), 2.22-2.34 (m, 3H), 2.68 (q, J=7.4 Hz, 2H, CH₂), 2.84 (m, 2H, H6), 4.25 (dd, J=11.8 and 9.1 Hz, 1H, CH₂NO₂), 4.48 (dd, J=11.8 and 6.0 Hz, 1H, CH₂NO₂), 4.93 (s, 2H, NH₂), 7.07 (s, 1H, ArH), 7.16 (s, 1H, ArH). ¹³C NMR (CDCl₃, 400 MHz): 13.1(CH₃), 15.1, 23.5, 24.4, 26.5, 26.9, 27.9, 29.5, 37.5, 38.6, 42.9, 44.4, 49.0, 54.8, 121.6, 127.2, 133.6, 136.0, 139.3, and 146.3. Microanalysis: C, 59.80 (expected 59.69); H, 7.27 (expected 7.16); N, 6.34 (expected 6.63).

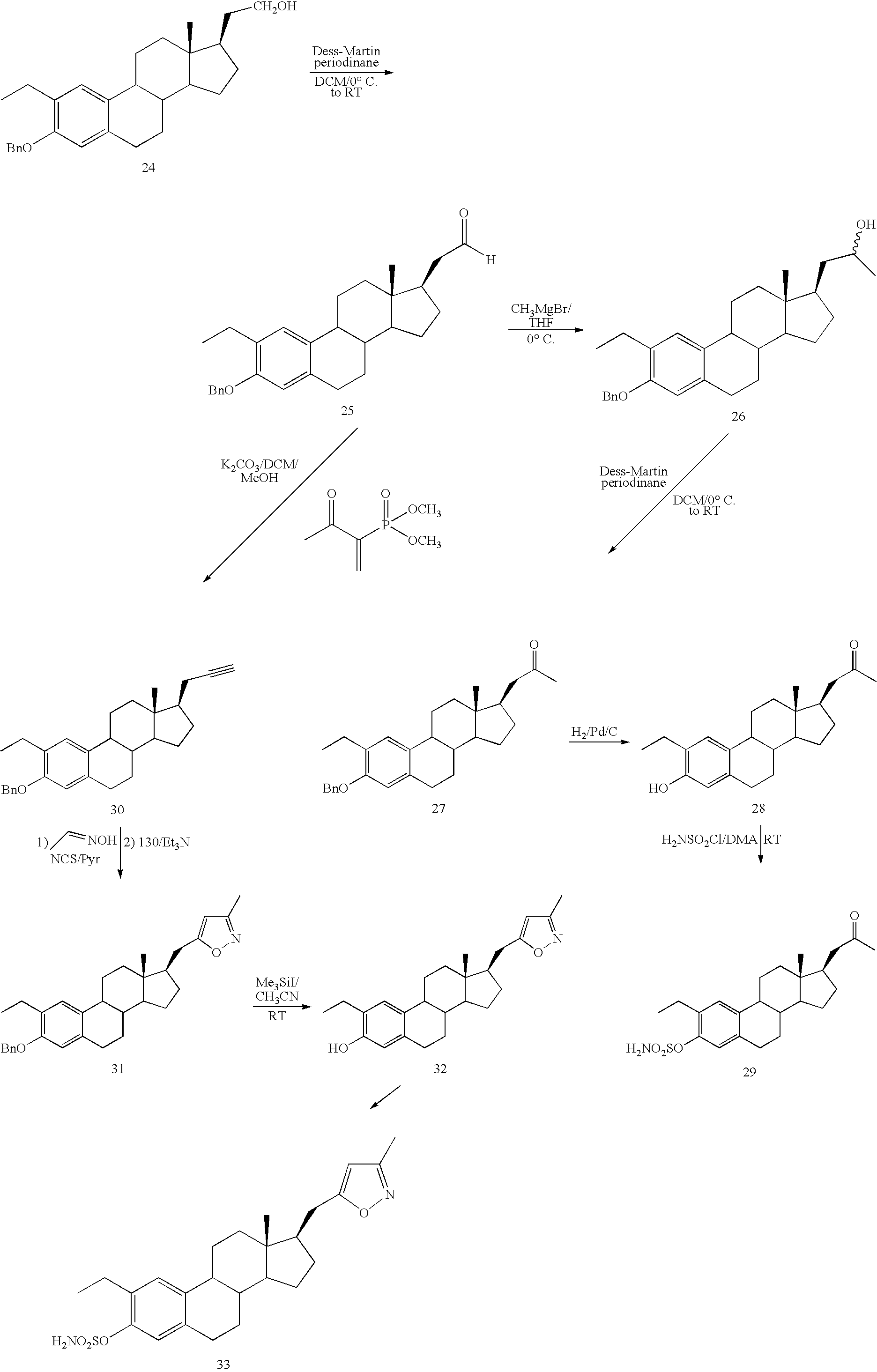

### 2-Ethyl-3-O-benzyl estrone-17β-(2-ethylaldehyde) 25

A solution of alcohol 24 (1.26 g, 3 mmol) in 50 ml DCM was cooled to 0° C. Dess Martin periodinane (1.4 g, 3.3 mmol) was added under nitrogen and the reaction mixture was stirred for 8 hours at 0° C. 100 ml of ether and 10 ml of a 1M aqueous sodium hydroxide solution were successively added and the mixture stirred for 30 minutes. The organic layer was successively washed with water and brine, dried over MgSO₄ and the solvents evaporated under educed pressure. The residual oil was purified by flash chromatography (hexane/ethyl acetate 50:1) to give 25 as a white solid, 165 mg (80%), mp=135-136° C.; ¹H NMR (CDCl₃, 400 MHz): 0.69 (s, 3H, CH₃), 1.26 (t, J=7.3 Hz, 3H, CH₃), 1.29-1.59 (m, 7H), 1.81-1.87 (m, 2H), 1.91-2.09 (m, 3H), 2.24-2.41 (m, 3H), 2.58 (ddd, J=15.7, 4.3 and 2.2 Hz, 1H, 1×H1′), 2.71 (q, J=7.3 Hz, 2H, CH₂), 2.88 (m, 2H, H6), 5.09 (s, 2H, CH₂Ph), 6.68 (s, 1H, ArH), 7.15 (s, 1H, ArH), 7.33-7.50 (m, 5H, 5H), 9.84 (dd, J=2.5 and 2.2 Hz, 1H, CHO); ¹³C NMR (CDCl₃, 100 MHz): 12.2 (CH₃), 14.6, 19.1, 23.4, 24.0, 26.4, 27.9, 28.4, 29.7, 37.9, 38.9, 42.5, 44.1, 45.6, 49.6, 54.6, 68.1 (C3′), 69.8, 84.5 (C2′), 111.8, 126.2, 127.0, 127.6, 128.4, 130.2, 132.5, 135.0, 137.7 and 154.5.

LR-MS: 417.30 (M+1) (expected: 417.28)

### 2-Ethyl-3-O-benzyl estrone-17β-(2-ethylaldehyde) 25

From 2-ethyl-3-O-benzyl-17β-cyanomethylestra-[1,3,5]-triene:

A solution of 2-ethyl-3-O-benzyl-17β-cyanomethylestra-[1,3,5]-triene- (0.85 g, 2.06 mmol) in THF (20 mL) was cooled to 0° C. A 1.5M solution of DIBAH (1.6 mL 2.4 mmol) was added in a dropwise manner under nitrogen and the reaction mixture was then stirred for 4 h at 0° C. After addition of a 2M aqueous solution of HCl (2 mL) and water (20 mL), the mixture was extracted with ethyl acetate and the organic layers were then washed with water and brine, dried and evaporated. The residual oil was purified by flash chromatography (hexane/ethyl acetate 30:1) to give 25 as a white solid, (490 mg, 57%), mp=135-136° C.

### 2-Ethyl-3-O-benzyl-17β-(2-hydroxypropyl) estrone 26

A solution of 25 (0.417 g, 1 mmol) in dry THF (20 ml) was cooled to −78° C. and then treated with a CH₃MgBr in Et₂O (0.5 ml, 1.5 mmol) in a drop wise manner. The solution was stirred at −78° C. for 2 hours then gradually worm to room temperature and stirred for 24 h. 10 ml of a saturated aqueous solution of ammonium chloride was added drop wise at 0° C. followed by 80 ml of ethyl acetate. The organic layer washed with water, brine, dried over MgSO₄ and the solvents evaporated under reduced pressure. The residual solid was purified by flash chromatography (hexane/ethyl acetate 50:1 to 15:1) to give 26 as a white powder 325 mg (78%), mp=66-69° C. (md) ¹H NMR (CDCl₃, 270 MHz): 0.61 and 0.627 (s, 3H, CH₃), 1.18-1.59 (m, 17H), 1.73-1.96 (m, 3H), 2.16-2.35 (m, 4H), 2.66 (q, J=7.4 Hz, 2H, CH₂), 2.83 (m, 2H, H6), 3.84 (m, 1H, CH(OH)), 5.03 (s, 2H, CH₂Ph), 6.63 (s, 1H, ArH), 7.11 (s, 1H, ArH), 7.27-7.46 (m, 5H, 5H). LR-MS: 433.37 and 433.43 (M+1) (expected: 433.31)

### 2-Ethyl-3-O-benzyl-17β-(2-oxopropyl) estrone 27

A solution of 26 (216 mg, 0.5 mmol) in 10 ml DCM stirred under nitrogen was cooled to 0° C. and 254 mg (0.6 mmol) of Dess-Martin Periodinane, were added portion wise. The solution was stirred for 4 hours at 0° C. before. 100 ml of diethyl ether were added as well as 1 ml of a 1M aqueous solution of sodium hydroxide. After 30 minutes stirring, the organic layer was washed with water, brine, dried over MgSO₄ and the solvents evaporated under reduced pressure. The residual oil was purified by flash chromatography (hexane/ethyl acetate 20:1) to give 27 as a white powder, 175 mg (81%), mp=46-47° C.; ¹H NMR (CDCl₃, 270 MHz): 0.65 (s, 3H, CH₃), 123 (t, J=7.3 Hz, 2H, CH₃), 1.26-1.58 (m, 7H), 1.65-2.05 (m, 5H), 2.18 (s, 3H, CH₃), 2.20-2.41 (m, 3H), 2.52-2.59 (m, 1H), 2.69 (q, J=7.3 Hz, 2H, CH₂), 2.85 (m, 2H, H6), 5.06 (s, 2H, CH₂Ph), 6.63 (s, 1H, ArH), 7.13 (s, 1H, ArH), 7.30-7.48 (m, 5H, 5H). ¹³C NMR (CDCl₃, 100 MHz): 12.8, 14.6, 23.4, 24.3, 26.4, 27.9, 28.4, 29.7, 30.2, 37.4, 38.9, 42.4, 44.0, 44.8, 46.0, 54.2, 69.7, 111.8, 126.1, 127.0, 127.5, 128.4, 130.1, 132.4, 135.0, 137.7, 154.4 and 209.5 (CO).

### 2-Ethyl-17β-(2-oxopropyl) estrone 28

A mixture of 27 (170 mg, 0.4 mmol) and 40 mg of 5% PD/C in 5 ml THF and 25 ml Ethanol was stirred under hydrogen for 16 hours. The suspension was filtered through a layer of celite/sand and the solvents removed under reduced pressure. The residual oil was purified by flash chromatography (hexane/ethyl acetate 20:1 to 15:1) to give 28 as a white powder, 120 mg (88%), mp-125-126° C.; ¹H NMR (CDCl₃, 400 MHz): 0.63 (s, 3H, CH₃), 1.23 (t, J=7.3 Hz, 2H, CH₃), 1.27-1.53 (m, 7H), 1.74-1.81 (m, 2H), 1.84-1.93 (m, 2H), 1.95-2.03 (m, 1H), 2.18 (s, 3H, CH₃), 2.16-2.24 (m, 1H), 2.26-2.33 (m, 1H), 2.52-2.58 (m, 1H), 2.60 (q, J=7.3 Hz, 2H, CH₂), 2.78 (m, 2H, H6), 4.99 (s, 1H, OH), 6.51 (s, 1H, ArH), 7.05 (s, 1H, ArH). ¹³C NMR (CDCl₃, 100 MHz): 12.8, 14.5, 23.1, 24.4, 26.5, 27.9, 28.5, 29.3, 30.4, 37.5, 38.9, 42.5, 44.1, 45.0, 46.1, 54.3, 115.3, 126.3, 127.2, 132.6, 135.5, 151.3 and 210.2 (CO). HRMS(FAB+): found 340.239357 for calcd. C₂₃H₃₂O₂ 340.240231

### 2-Ethyl-3-O-sulfamoyl-17β-(2-oxopropyl) estrone 29

A solution of sulfamoyl chloride (0.6 mmol) in DMA (1 ml) cooled to 0° C. was added to 28 (102 mg, 0.3 mmol) and the mixture was stirred for 24 hours at room temperature under nitrogen. After addition of water (10 ml) the organics were extracted with ethyl acetate (2×50 ml). The organic layer was successively washed with water, brine and dried over MgSO₄. The solvent was removed under vacuum and the residual solid was purified by flash chromatography (hexane/ethyl acetate 10:1 to 4:1) and recrystallised in hexane/ethyl acetate 6:1 to give 29 as a white powder, 108 mg (86%), mp=204-205° C. ¹H NMR (CDCl₃/CD₃COCD₃ 4:1: 400 MHz): 0.40 (s, 3H, CH₃), 0.94 (t, J=7.3 Hz, 2H, CH₃), 0.95-1.33 (m, 7H), 1.51-1.77 (m, 5H), 1.91 (s, 3H, CH₃CO), 1.99-2.12 (m, 3H), 2.28-2.34 (m, 1H), 2.45 (q, J=7.3 Hz, 2H, CH₂), 2.56 (m, 211, H6), 6.27 (s, 2H, NH₂), 6.84 (s, 1H, ArH), 6.92 (s, 1H, ArH). ¹³C NMR (CDCl₃/CD₃COCD₃, 4:1, 100 MHz): 12.2, 14.2, 22.8, 24.1, 26.2, 27.6, 28.2, 28.4, 29.3, 37.3, 38.7, 42.1, 44.1, 44.3, 46.0, 54.3, 121.8, 126.7, 127.2, 132.6, 133.5, 138.7, 145.7 and 207.5 (CO). HRMS (FAB+): found 419.212303 for calcd. C₂₃H₃₃NO₄S 419.213031.

### 2-Ethyl-3-O-benzyl-17β-(prop-2-ynyl) estrone 30

A mixture of (1-Diazo-2-oxo-propyl)-phosphonic acid dimethyl ester (0.29 g, 1.5 mmol) and dry K₂CO₃ in 2.5 ml dry methanol was stirred under nitrogen and cooled to 0° C. before 25 (208 mg, 0.5 mmol) in 2 ml DCM was added drop wise. The mixture was stirred for 24 hours at room temperature, water (10 ml) and DCM (50 ml) added to the solution and the organic layer washed with water and brine successively, dried over MgSO₄. After evaporation of the solvent under reduced pressure, the resulting oil was purified by flash chromatography (hexane/ethyl acetate 25:1) to give 30 as a white solid, 165 mg (80%), mp=78-79° C. ¹H NMR (CDCl₃, 270 MHz): 0.67 (s, 3H, CH₃), 1.22 (t, J=7.3 Hz, 3H, CH₃), 1.24-1.59 (m, 8H), 1.66-1.80 (m, 2H), 1.87-2.14 (m, 4H), 2.15-2.38 (m, 3H), 2.70 (q, J=7.3 Hz, 2H, CH₂), 2.86 (m, 2H, H6), 5.01 (s, 2H, CH₂Ph), 6.66 (s, 1H, ArH), 7.14 (s, 1H, ArH), 7.30-7.49 (m, 5H, 5H). ¹³C NMR (CDCl₃, 67.5 MHz): 12.2 (CH₃), 14.6, 19.1, 23.4, 24.0, 26.4, 27.9, 28.4, 29.7, 37.9, 38.9, 42.5, 44.1, 45.6, 49.6, 54.6, 68.1 (C3′), 69.8, 84.5 (C2′), 111.8, 126.2, 127.0, 127.6, 128.4, 130.2, 132.5, 135.0, 137.7 and 154.5. LR-MS: 413.40 (M+1) (expected: 413.28).

### 2-Ethyl-3-O-benzyl-17β-(3-methyl-isoxazol-5-ylmethyl)-17-deoxy estrone 31

A mixture of NCS (0.8 g, 6 mmol) and pyridine (0.08 ml, 1 mmol) in 10 ml CHCl₃ was stirred at room temperature under nitrogen and acetaldoxime (354 mg, 6 mmol) added portion wise. After 15 minutes 30 (0.825 g, 2 mmol) in pyridine (2 ml) was added in a dropwise manner followed by Et₃N (0.91 ml, 8 mmol). The mixture was refluxed for 24 hours. The solvents were then evaporated under reduced pressure and the residual oil was dissolved in 100 ml ethyl acetate. The organic layer washed with water, brine, dried over MgSO4 and the solvents evaporated under reduce pressure. The residual oil was purified by flash chromatography (hexane/ethyl acetate 50:1 to 35:1) to give 380 mg (46%) of recovered starting material 30 together with 350 mg (37%) of the desired isoxaole 31 as a white powder, mp=128-129° C. ¹H NMR (CDCl₃, 270 MHz): 0.70 (s, 3H, CH₃), 1.21 (t, J=7.3 Hz, 3H, CH₃), 1.22-1.97 (m, 12H), 2.18-2.33(s+m, 5H, 2H+CH₃), 2.54 (dd, J=15.1 and 9.4 Hz, 1H, H1′), 2.67 (q, J=7.3 Hz, 2H, CH₂), 2.78-2.86 (m, 3H, H6 and H1′), 5.04 (s, 2H, CH₂Ph), 5.81 (s, 1H, H-isoxazole), 6.63 (s, 1H, ArH), 7.10 (s, 1H, ArH), 7.28-7.46 (m, 5H, 5H). ¹³C NMR (CDCl₃, 67.5 MHz): 11.5, 12.5 (CH₃), 14.7, 23.5, 24.3, 26.5, 27.7, 27.9, 28.6, 29.8, 37.6, 38.9, 42.7, 44.1, 49.1, 54.5, 69.9, 101.8, 111.9, 126.3, 127.1, 127.7, 128.5, 130.2, 132.4, 135.0, 137.7, 154.5, 159.7 and 173.3. LR-MS: 470.35 (M+1) (expected: 470.31)

### 2-Ethyl-17β-(3-methyl-isoxazol-5-ylmethyl)-17-deoxy estrone 32

A mixture of TMSCl (0.1 ml, 1.1 mmol) and sodium iodide (165 mg, 1.1 mmol) in 5 ml dry acetonitrile was stirred for 30 minutes at room temperature under nitrogen. 31 (235 mg, 0.5 mmol) in 2 ml dry acetonitrile was then added drop wise and the mixture stirred at room temperature for 6 hours. 80 ml of ethyl acetate were added and the organic layer was successively washed with a 1M solution of sodium thiosulfate, water and brine, dried over magnesium sulfate before the solvents were removed under reduced pressure. The resulting oil was purified by flash chromatography (hexane/ethyl acetate 10:1 to 8:1) to give the desired alcohol 32 as a white solid, 170 mg, (89%), mp=195-196° C.; ¹H NMR (CDCl₃, 270 MHz): 0.70 (s, 3H, CH₃), 1.21 (t, J=7.3 Hz, 3H, CH₃), 1.20-1.97 (m, 12H), 2.18-2.33(s+m, 5H, 2H+CH₃), 2.51 (dd, J=15.1 and 9.4 Hz, 1H, H1′), 2.60 (q, J=7.3 Hz, 2H, CH₂), 2.75-2.88 (m, 3H, H6 and H1′), 4.95 (s, 1H, OH), 5.81 (s, 1H, H-isoxazole), 6.51 (s, 1H, ArH), 7.05 (s, 1H, ArH). LR-MS: 379.78 (M+1) (expected: 379.25).

### 2-Ethyl-3-O-sulfamoyl-17β-(3-methyl-isoxazol-5-ylmethyl)-estra-[1,3,5]-triene 33

An ice cold solution of sulfamoyl chloride (0.35 mmol) in DMA (1 mL) was treated with 32 (60 mg, 0.16 mmol). After 16 h at room temperature water (5 mL) was added and the mixture was then extracted with ethyl acetate (2×50 mL). The combined organic layers were washed with water, brine, dried (MgSO₄) and evaporated. The residual solid was purified by flash chromatography (hexane/ethyl acetate 6:1 to 3:1) to give 33 as a white powder. White solid, 45 mg, (63%), mp=103-104° C.; ¹H NMR (270 MHz, CDCl₃): 0.70 (s, 3H, CH₃), 1.21 (t, J=7.3 Hz, 3H, CH₃), 1.24-1.55 (m, 7H), 1.70-1.77 (m, 2H), 1.81-1.97 (m, 3H), 2.14-2.32 (s+m, 5H, 2H+CH₃), 2.55 (dd, J=14.9 and 9.8 Hz, 1H, H1′), 2.59 (q, J=7.3 Hz, 2H, CH₂), 2.78 (m, 2H, H6), 2.82 (dd, J=14.9 and 4.5 Hz, 1H, H1′), 4.67 (s, 1H, OH), 5.81 (s, 1H, Hisoxazole), 6.50 (s, 1H, ArH), 7.04 (s, 1H, ArH); ¹³C NMR (CDCl3, 67.5 MHz): 11.5, 12.4 (CH₃), 14.5, 23.1, 24.2, 26.5, 27.7, 27.8, 28.5, 29.3, 37.5, 38.9, 42.6, 44.1, 49.1, 54.5, 101.8, 115.2, 126.3, 127.2, 132.6, 135.5, 151.2, 159.7 and 173.3; LR-MS: 380.22 (M+1) (expected: 380.26); HRMS(FAB+): calcd. for C₂₅H₃₄O₄N₂S 458.223930 found 458.224014

### 2-Methoxy estrone 17-(4H-[1,2,4]-triazol-4-ylamino) imine 34

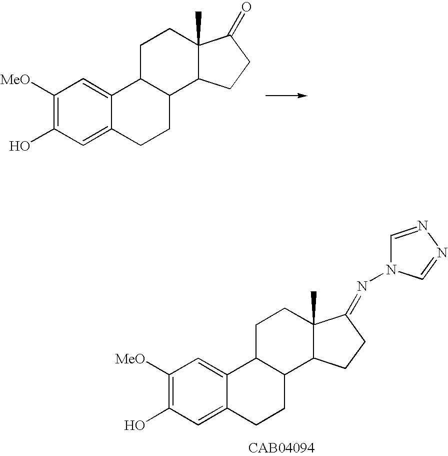

A solution of 2-methoxy-estrone (1.00 g, 3.33 mmol), 4-amino-4H-1,2,4-triazole (560 mg, 6.66 mmol) and pTsOH hydrate (50 mg) in EtOH (5 ml) was heated in an ACE-pressure tube to 100° for 20 hours. After cooling to r.t. a white crystalline solid was filtered off, washed with a small amount of cold EtOH (ca 5 ml) and dried under high vacuum to give 34 (877 mg, 72%) as fine colourless needles. ¹H NMR (400 MHz, DMSO-d₆) δ 1.03 (s, 3H, H-18), 1.24-1.73 (m, 6H), 1.8-1.96 (m, 2H), 2.02-2.08 (m, 1H), 2.19-2.27 (m, 1H), 2.36-2.48 (m, 2H), 2.62-2.78 (m, 2H), 2.86-2.94 (m, 1H), 3.74 (s, 3H, —OCH₃), 6.48 (s, 1H), 6.81 (s, 1H), 8.67 (s, 1H, —OH), 8.76 (s, 2H); 2.86 (FAB+): m/z 298.0 (50%), 367.0 (100%), [C₂₁H₂₇N₄O₂]⁺); HRMS (FAB+) for C₂₁H₂₇N₄O₂₄: 367.2134; found, 367.2144.

### 2-Methoxy-17β-(4H-[1,2,4]-triazol-4-ylamino)-17-deoxy estrone 35

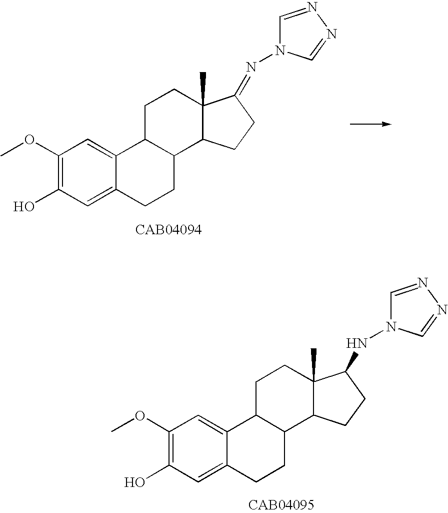

Sodium borohydride (38 mg, 1.00 mmol) was added to a solution of the imine 34 (110 mg, 0.30 mmol) in MeOH (10 ml) at 0° C. The clear solution was stirred for 2 hours at this temperature, then water (50 ml) and EtOAc (50 ml) were added. The organic layer was separated, washed with water (20 ml) and brine (20 ml), dried over Na₂SO₄ and concentrated under reduced pressure. The residue was dissolved in EtOAc and precipitated by addition of Et₂O to give 35 as a white solid (69 mg, 62%). ¹H NMR (270 MHz, DMSO-d₆) δ 0.78 (s, 3H, H-18), 1.00-1.54 (m, 7H), 1.60-1.92 (m, 4H), 1.98-2.22 (m, 2H), 2.54-2.78 (m, 2H), 3.12-3.24 (m, 1H, H-17), 3.68 (s, 3H, —OCH₃), 6.42 (s, 1H), 6.64-6.74 (m, 2H), 8.58 (s, 1H), 8.61 (s, 2H); MS (FAB+): m/z 369.1 (100%), [C₂₁H₂₉N₄O₂]⁺).

### 2-Methoxy-3-O-sulfamoyl estrone 17-(4H-[1,2,4]-triazol-4-ylamino) imine 36

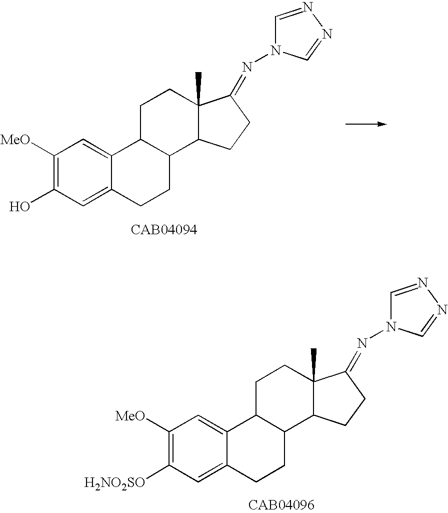

Sulfamoyl chloride solution in toluene (7 ml, 0.7 M, 4.9 mmol) was concentrated under reduced pressure (30° C. water bath temperature) to ca. 0.5 ml volume. The residue was cooled to 0° C. (ice bath) and N,N-dimethyl acetamide (5 ml) was added. Imine 34 (550 mg, 1.50 mmol) was added to the colourless solution and the mixture was stirred for 18 hours at room temperature. Ethyl acetate (70 ml) and water (50 mL) were added to the solution, the organic layer was separated, washed with water (2×30 ml) and brine (1×20 ml), dried over Na₂SO₄ and concentrated under reduced pressure. The residue was dissolved in a small amount of acetone and precipitated by addition of Et₂O. The precipitate was filtered off and dried under high vacuum to yield 36 (568 mg, 85%) as a white solid. ¹H NMR (400 MHz, DMSO-d₆) δ 1.06 (s, 3H, H-18), 1.24-1.78 (m, 7H), 1.88-1.98 (m, 1H), 2.04-2.14 (m, 1H), 2.27-2.50 (m, 2H), 2.76-2.84 (m, 2H), 2.88-2.98 (m, 1H), 3.80 (s, 3H, —OCH₃), 7.03 (s, 1H), 7.04 (s, 1H), 7.85 (s, 2H, —NH₂), 8.79 (s, 2H); ¹³C NMR (100.5 MHz, CDCl₃) δ 16.9, 23.2, 26.1, 26.9, 28.6, 29.2, 31.4, 34.0, 37.9, 44.4, 46.8, 51.1, 56.4, 111.0, 123.5, 128.7, 137.4, 138.9, 140.5, 150.0, 189.0;

MS (FAB+): m/z 446.0 (100%, [C₂₁H₂₇N₅O₄S+H]⁺); HRMS (FAB+) for C₂₁H₂₈N₅O₄S: 446.1862; found, 446.1884.

### 2-Methoxy-17β-(4H-[1,2,4]-triazol-4-ylamino)-17-deoxy estrone-3-O-sulfamate 37

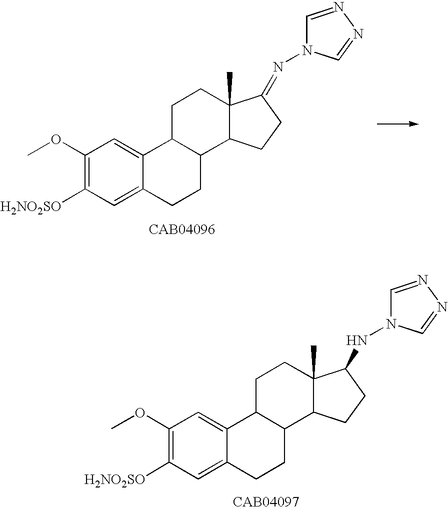

Sodium borohydride (38 mg, 1.00 mmol) was added to a solution of the sulfamoylated imine 36 (143 mg, 0.32 mmol) in MeOH (10 ml) at 0° C. The clear solution was stirred for 2 hours at this temperature, then water (50 ml) and EtOAc (50 ml) were added. The organic layer was separated, washed with water (20 ml) and brine (20 ml), dried over Na₂SO₄ and concentrated under reduced pressure. The residue was dissolved in EtOAc and precipitated by addition of Et₂O to yield 37 (81 mg, 57%) as a white solid. ¹H NMR (270 MHz, DMSO-d₆) δ 0.79 (s, 3H, H-18), 1.02-1.54 (m, 7H), 1.60-1.92 (m, 4H), 2.04-2.22 (m, 2H), 2.66-2.78 (m, 2H), 3.14-3.24 (m, 1H, H-17), 3.73 (s, 3H, —OCH₃), 6.71 (d, J=1.7 Hz, 1H), 6.93 (s, 1H), 6.95 (s, 1H), 7.80 (s, 2H, —NH₂), 8.61 (s, 2H); MS (FAB+): m/z 447.9 (100%), [C₂₁H₂₉N₅O₄S]⁺).

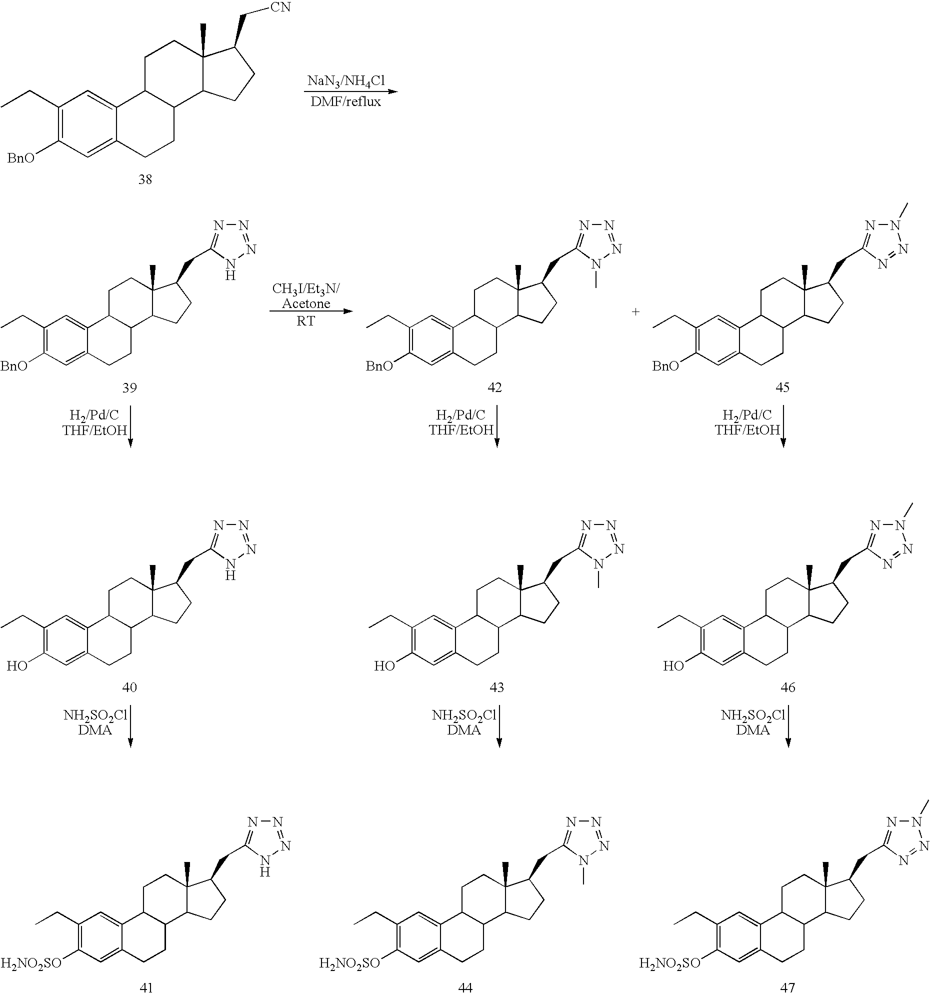

### 2-Ethyl-3-O-benzyl-17β-(1H-tetrazol-5-ylmethyl)-17-deoxyestrone 39

A mixture of 2-ethyl-3-O-benzyl-17-(cyanomethyl) estrone 38 (0.83 g, 2 mmol), sodium azide (0.26 g, 4 mmol) and ammonium chloride (214 mg, 4 mmol) in 10 ml DMF was refluxed for 1 day. 0.13 g (μmol) of sodium azide was added and the mixture was refluxed another 24 hours. This was repeated 3 times and after an overall 5 days reflux the mixture was cooled to room temperature. Water (50 ml) and ethyl acetate (100 ml) were added and the organic layer washed successively with water, brine, dried over MgSO4. The solvents were removed under reduced pressure and the residual solid was purified by flash chromatography (hexane/ethyl acetate 5:1 to 3:2) to give 39 as a white solid (0.78 g, 85%), mp=214-215° C. which showed ¹H NMR (CD₃COCD₃, 270 MHz): 0.79 (s, 3H, CH₃), 1.15 (t, J=7.4 Hz, 2H, CH₃), 1.22-1.48 (m, 6H), 1.60-1.66 (m, 1H), 1.72-2.01 (m, 5H), 2.12-2.34 (m, 2H), 2.62 (q, J=7.4 Hz, 2H, CH₂), 2.78 (m, 2H, H6), 2.84 (dd, J=14.9 and 5.4 Hz, 1H, H1′), 3.10 (dd, J=14.9 and 5.7 Hz, 1H, H1′), 5.07 (s, 2H, CH₂Ph), 6.70 (s, 1H, ArH), 7.06 (s, 1H, ArH), 7.28-7.50 (m, 5H, 5H). ¹³C NMR (CD₃COCD₃, 100 MHz): 11.8, 14.3, 23.3, 23.9, 24.0, 26.3, 27.8, 28.1, 29.5, 37.4, 39.0, 44.1, 49.4, 54.5, 69.4, 111.9, 126.1, 127.2, 127.6, 128.4, 129.6, 132.1, 134.8, 138.1 and 154.4, 164.7. LRMS: 456.18 (expected 456.29)

### 2-Ethyl-17β-(1H-tetrazol-5-ylmethyl)-17-deoxyestrone 40

To a solution of 39 (228 mg, 0.5 mmol) in THF (5 ml) and ethanol (15 ml) was added 50 mg of 5% Pd/C and the mixture was stirred under hydrogen for 48 hours. After filtration through celite/sand, the organics were concentrated under reduced pressure and the residual solid was purified by flash chromatography (hexane/ethyl acetate 4:1 to 1:1) and recrystallisation. The product 40 a white powder (155 mg, 85%), mp=248-249° C. showed ¹H NMR (CD₃COCD₃, 270 MHz): 0.81 (s, 3H, CH₃), 1.15 (t, J=7.4 Hz, 2H, CH₃), 1.20-1.47 (m, 6H), 1.60-1.66 (m, 1H), 1.72-2.01 (m, 5H), 2.12-2.34 (m, 2H), 2.60 (q, J=7.4, 2H, CH₂), 2.78 (m, 2H, H6), 2.94 (dd, J=14.5 and 5.5, 1H, H1′), 3.10 (dd, J=14.9 and 5.8, 1H, H1′), 6.54 (s, 1H, ArH), 7.02 (s, 1H, ArH). ¹³C NMR (CD₃COCD₃, 100 MHz): 11.2, 13.6, 22.5, 23.3, 25.4, 25.8, 27.2, 27.6, 29.0, 37.0, 38.5, 41.8, 43.5, 49.2, 53.9, 114.1, 125.4, 126.8, 130.3, 133.9, 151.9 and 164.9.

### 2-Ethyl-17β-(1-methyl-1H-tetrazol-5-ylmethyl)-17-deoxyestrone 42 and 2-Ethyl-17β-(2-methyl-2H-tetrazol-5-ylmethyl)-17-deoxyestrone 45

A solution of 39 (456 mg, 1 mmol), methyl iodide (0.12 ml, 2 mmol) and triethylamine (0.28 ml, 2 mmol) in 10 ml acetone was stirred at room temperature for 5 hours. After addition of 20 ml water, the organics were extracted with ethyl acetate (2×50 ml) and the organic layer washed with water, brine, dried over magnesium sulfate. The solvents were evaporated under reduced pressure and the residual solid purified by flash chromatography (hexane/ethyl acetate 10:1 to 3:1) to give 2-Ethyl-17β-(1-methyl-1H-tetrazol-5-ylmethyl)-17-deoxyestrone 42 as a white powder, 195 mg (42%), mp=144-145° C.; ¹H NMR (CDCl₃, 270 MHz): 0.74 (s, 3H, CH₃), 1.19 (t, J=7.4 Hz, 2H, CH₃), 1.21-1.51 (m, 7H), 1.60-2.03 (m, 6H), 2.15-2.34 (m, 2H), 2.62 (q, J=7.4 Hz, 2H, CH₂), 2.66 (dd, J=14.4 and 9.7 Hz, 1H, H1′), 2.81 (m, 2H, H6), 2.99 (dd, J=14.9 and 5.0 Hz, 1H, H1′), 4.23 (s, 3H, CH₃N), 5.03 (s, 2H, CH₂Ph), 6.61 (s, 1H, ArH), 7.09 (s, 1H, ArH), 7.26-7.45 (m, 5H, 5H). ¹³C NMR (CDCl₃, 100 MHz): 12.5, 14.7, 23.5, 24.2, 26.2, 26.5, 27.9, 28.4, 29.8, 37.6, 39.0, 39.2, 42.7, 44.1, 49.6, 54.5, 69.9, 111.9, 126.2, 127.1, 127.6, 128.5, 130.2, 132.5, 135.1, 137.8 and 154.5, 167.0. LRMS: 470.89 (expected 470.30) and 2-Ethyl-17β-(2-methyl-2H-tetrazol-5-ylmethyl)-17-deoxyestrone 45 as a white powder, 155 mg (33%), mp=125-126° C.; ¹H NMR (CDCl₃, 270 MHz): 0.69 (s, 3H, CH₃), 1.13 (t, J=7.3 Hz, 2H, CH₃), 1.15-1.52 (m, 7H), 1.59-1.97 (m, 5H), 2.09-2.27 (m, 2H), 2.58 (q, J=7.3 Hz, 2H, CH₂,), 2.60 (m, 1H, H1′), 2.74 (m, 2H, H6), 2.87 (dd, J=14.9 and 4.6 Hz, 1H, H1′), 3.91 (s, 3H, CH₃N), 4.95 (s, 2H, CH₂Ph), 6.55 (s, 1H, ArH), 7.00 (s, 1H, Arm), 7.26-7.40 (m, 5H, 5H). ¹³C NMR (CDCl₃, 100 MHz): 12.6, 14.7, 23.5, 23.9, 24.2, 26.4, 27.8, 28.4, 29.7, 33.4, 37.4, 38.9, 42.8, 44.0, 48.6, 54.3, 69.9, 111.9, 126.2, 127.1, 127.7, 128.5, 130.3, 132.2, 135.0, 137.8 and 154.5, 155.1. LRMS: 470.89 (expected 470.30).

### 2-Ethyl-3-O-sulfamoyl-17β-((1H-tetrazol-5-yl)methyl)-7estra-[1,3,5]-triene 41

To a ice cold solution of sulfamoyl chloride (0.87 mmol) in DMA (1 mL) was added 40 (80 mg, 0.22 mmol). After 16 hours stirring at room temperature water (5 mL) was added and the mixture was then extracted with ethyl acetate (2×50 mL). The combined organic layers washed with water, brine, dried and evaporated. The residual solid was purified by flash chromatography (hexane/ethyl acetate 1:1) to give 41 as a white powder. White powder, 50 mg (52%), mp=223-224° C.; ¹H NMR (CD₃COCD₃, 400 MHz): 0.68 (s, 3H, CH₃), 1.04 (t, J=7.3 Hz, 2H, CH₃), 1.08-1.40 (m, 7H), 1.49-1.55 (m, 1H), 1.62-1.80 (m, 3H), 1.82-1.94 (m, 2H), 2.07-2.14 (m, 1H), 2.18-2.24 (m, 1H), 2.56 (q, J=7.3 Hz, 2H, CH₂), 2.66-2.76 (m, 3H, H6+H1′), 2.98 (dd, J=14.8 and 5.9 Hz, 1H, H1′), 6.95 (s, 1H, ArH), 7.01 (s, 1H, ArH); ¹³C NMR (CD₃COCD₃, 100 MHz): 11.8, 14.2, 22.8, 23.9, 26.1, 27.5, 28.0, 28.9, 29.2, 37.3, 38.6, 42.5, 44.2, 49.4, 54.4, 121.8, 126.7, 133.9, 135.4, 138.6, 146.6 and 164.9. Microanalysis: C, 59.00 (expected 59.30); H, 7.05 (expected 7.01); N: 15.50 (expected 15.72).

### 2-Ethyl-3-hydroxy-17β-((1-methyl-1H-tetrazol-5-yl)methyl)-estra-[1,3,5]-triene 43

To a solution of 42 (110 mg, 0.23 mmol) in THF (5 ml) and ethanol (15 ml) was added 5% Pd/C (30 mg) and the mixture was then stirred under an atmosphere of hydrogen for 24 h. After filtration through celite/sand, the organics were concentrated under reduced pressure and the residual solid was purified by flash chromatography (hexane/ethyl acetate 8:1) and recrystallisation (hexane/diethyl ether 2:1) to give 43 as pale yellow needles, 80 mg (90%), mp=206-207° C.; ¹H NMR (270 MHz, CDCl₃): 0.76 (s, 3H, CH₃), 1.22 (t, J=7.3 Hz, 2H, CH₃), 1.24-1.55 (m, 7H), 1.65-2.03 (m, 5H), 2.14-2.32 (m, 2H), 2.60 (q, J=7.3 Hz, 2H, CH₂), 2.67 (dd, J=14.8 and 5.2 Hz, 1H, H1′), 2.73-2.82 (m, 2H, H6), 2.96 (dd, J=14.8 and 4.5 Hz, 1H, H1′), 4.02 (s, 3H, CH₃N), 5.18 (s, 1H, OH), 6.53 (s, 1H, ArH), 7.03 (s, 1H, ArH); ¹³C NMR (CDCl₃, 100 MHz): 12.5, 14.4, 23.0, 23.8, 24.1, 26.3, 27.7, 28.3, 29.2, 33.4, 37.3, 38.8, 42.7, 43.9, 48.5, 54.2, 115.2, 126.2, 127.3, 132.2, 135.3, 151.3 and 155.0. LRMS (FAB+): 381.47 (expected 381.27) HRMS(FAB+): calculated for calcd. C₂₃H₃₂ON₄ 380.257612, found 380.256607.

### 2-Ethyl-3-O-sulfamoyl-17β-((1-methyl-1H-tetrazol-5-yl)methyl)-estra-[1,3,5]-triene 44

An ice cold solution of sulfamoyl chloride (0.26 mmol) in DMA (1 mL) was treated with 43 (50 mg, 0.13 mmol). After 16 h at room temperature water (5 mL) was added and the mixture was then extracted with ethyl acetate (2×50 mL). The combined organic layers were washed with water, brine, dried (MgSO₄) and evaporated. The residual solid was purified by flash chromatography (hexane/ethyl acetate 1:1 to 1:3) to give 44 as a white powder. White solid, 30 mg (51%), mp=218-219° C.; ¹H NMR (270 MHz, CDCl₃): 0.76 (s, 3H, CH₃), 1.19 (t, J=7.3 Hz, 2H, CH₃), 1.20-1.51 (m, 7H), 1.54-2.01 (m, 8H), 2.12-2.30 (m, 2H), 2.67 (q, J=7.3 Hz, 2H, CH₂), 2.70 (dd, J=14.9 and 5.3 Hz, 1H, H1′), 2.81 (m, 2H, H6), 2.95 (dd, J=14.9 and 4.6 Hz, 1H, H1′), 4.00 (s, 3H, CH₃N), 5.05 (s, 2H, NH₂), 7.06 (s, 1H, ArH), 7.15 (s, 1H, ArH); ¹³C NMR (CDCl₃, 100 MHz): 12.5, 14.7, 23.1, 23.9, 24.2, 26.2, 27.5, 28.4, 29.2, 33.5, 37.3, 38.4, 42.7, 44.2, 48.6, 54.3, 121.5, 126.9, 133.7, 136.0, 139.3, 146.2 and 155.0.

Microanalysis: C, 59.60 (expected 60.11); H, 7.22 (expected 7.24); N, 15.20 (expected 15.24).

### 2-Ethyl-3-hydroxy-17β-((2-methyl-2H-tetrazol-5-yl)methyl)-estra-[1,3,5]-triene 46

To a solution of 45 (165 mg, 0.35 mmol) in THF (5 ml) and ethanol (15 ml) was added 5% Pd/C (30 mg) and the mixture was stirred under hydrogen for 24 h. After filtration through celite/sand, the organics were concentrated under reduced pressure and the residual solid was purified by flash chromatography (hexane/ethyl acetate 8:1) and recrystallisation (hexane/diethyl ether 2:1) to give 46 as a white powder, 155 mg (85%), mp=117-118° C.; ¹H NMR (CD₃COCD₃, 270 MHz): 0.73 (s, 3H, CH₃), 1.19 (t, J=7.3 Hz, 2H, CH₃), 1.21-1.53 (m, 7H), 1.58-2.00 (m, 5H), 2.11-2.29 (m, 2H), 2.58 (q, J=7.3 Hz, 2H, CH₂), 2.71 (dd, J=14.6 and 9.9 Hz, 1H, H1′), 2.76 (m, 2H, H6), 2.97 (dd, J=14.6 and 5.0 Hz, 1H, H1′), 4.29 (s, 3H, CH₃N), 4.73 (s, 1H, OH), 6.48 (s, 1H, ArH), 7.02 (s, 1H, ArH); ¹³C NMR (CD₃COCD₃, 100 MHz): 12.5, 14.4, 23.1, 24.2, 26.2, 26.5, 27.8, 28.3, 29.3, 37.6, 38.9, 39.3, 42.7, 44.1, 49.6, 54.5, 115.2, 126.3, 127.2, 132.7, 135.5, 151.2 and 167.0. LRMS (FAB+): 381.31 (expected 381.27)

### 2-Ethyl-3-O-sulfamoyl-17β-((2-methyl-2H-tetrazol-5-yl)methyl)-estra-[1,3,5]-triene 47

An ice cold solution of sulfamoyl chloride (0.26 μmmol) in DMA (1 mL) was treated with 46 (50 mg, 0.13 mmol). After 16 h at room temperature water (5 mL) was added and the mixture was then extracted with ethyl acetate (2×50 mL). The combined organic layers were washed with water, brine, dried (MgSO₄) and evaporated. The residual solid was purified by flash chromatography (hexane/ethyl acetate 1:1 to 1:3) to give 47 as a white powder. White solid, 55 mg (90%), mp=86-87° C.; ¹H NMR (270 MHz, CDCl₃): 0.67 (s, 3H, CH₃), 1.13 (t, J=7.3 Hz, 2H, CH₃), 1.19-1.44 (m, 7H), 1.60-1.96 (m, 8H), 2.09-2.24 (m, 2H), 2.61 (q, J=7.3 Hz, 2H, CH₂), 2.65 (dd, J=14.6 and 9.7 Hz, 1H, H1′), 2.75 (m, 2H, H6), 2.92 (dd, J=14.6 and 5.0 Hz, 1H, H1′), 4.23 (s, 3H, CH₃N), 5.01 (br, 2H, NH₂), 7.00 (s, 1H, ArH), 7.10 (s, 1H, ArH). ¹³C NMR (CDCl₃, 100 MHz): 12.5, 14.6, 23.1, 24.2, 26.2, 26.3, 27.6, 28.3, 29.2, 37.5, 38.4, 39.3, 42.6, 44.3, 49.5, 54.5, 121.4, 127.0, 133.6, 136.0, 139.7, 146.1 and 166.9.

LRMS (FAB+): (M+1) 460.27 (expected 460.24)

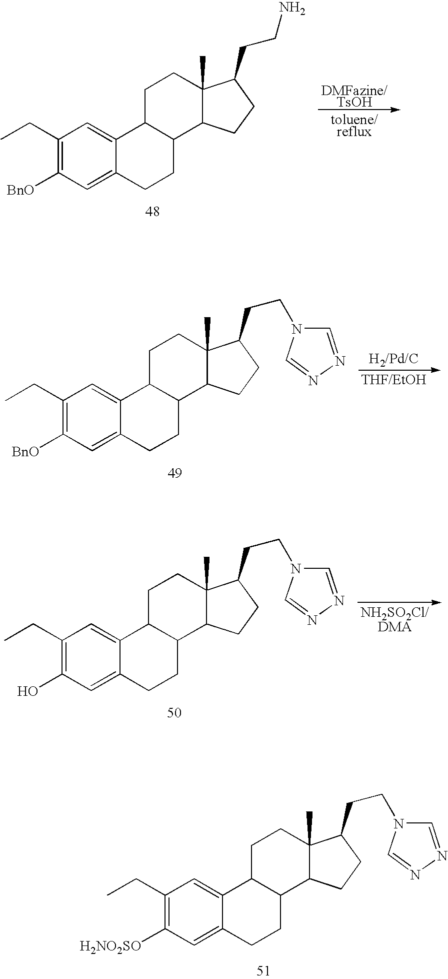

### 2-Ethyl 3-O-benzyl 17β-([1,2,4]triazol-4-yl-ethyl)-17-deoxy estrone 49

A solution of 2-ethyl 3-O-benzyl 17β-(2-aminoethyl)-17-deoxy estrone 48 (413 mg, 1 mmol) and p-TsOH.H₂O (19 mg, 0.1 mmol) in 30 ml toluene was refluxed for 24 hours and the solvent was removed under reduced pressure. The residual solid was extracted with ethyl acetate and the organic layer washed with water, brine, dried over magnesium sulfate before the solvent was removed under reduced pressure. The residual solid was purified by flash chromatography (ethyl acetate/methanol 1:0 to 30:2) to give the desired triazole derivative 49 as a white solid, 340 mg (73%), mp=203-204° C.; ¹H NMR (CDCl₃, 270 MHz): 0.63 (s, 3H, CH₃), 1.20 (t, J=7.3 Hz, 2H, CH₃), 1.24-1.70 (m, 9H), 1.75-1.99 (m, 5H), 2.15-2.35 (m, 2H), 2.6 (q, J=7.3 Hz, 2H, CH₂), 2.80 (m, 2H, H6), 3.90-4.10 (m, 2H, H2′), 5.02 (s, 2H, CH₂Ph), 6.62 (s, 1H, ArH), 7.08 (s, 1H, ArH), 7.26-7.45 (m, 5H, 5H), 8.16 (s, 2H, triazole). ¹³C NMR (CDCl₃, 100 MHz): LRMS (M+1)⁺: 470.38 (expected 470.32).

### 2-Ethyl-3-hydroxy-17β-(2-[1,2,4]triazol-4-yl-ethyl)-estra-[1,3,5]-triene 50

A solution of 49 (234 mg, 0.50 mmol) in THF (5 ml) and ethanol (15 ml) was treated with 5% Pd/C (50 mg) and then placed under an atmosphere of hydrogen for 24 h. After filtration through celite/sand, the organics were concentrated under reduced pressure and the residual solid was recrystallized in ethanol/water 10:1. White powder, 150 mg (79%), mp=257-258° C.; ¹H NMR (CD₃OD, 270 MHz): 0.67 (s, 3H, CH₃), 1.13 (t, J=7.3 Hz, 2H, CH₃), 1.15-1.49 (m, 8H), 1.60-2.16 (m, 7H), 2.25-2.32 (m, 1H), 2.53 (q, J=7.3 Hz, 2H, CH₂), 2.71 (m, 2H, H6), 4.03-4.22 (m, 2H, H2′), 6.41 (s, 1H, ArH), 6.93 (s, 1H, ArH), 8.57 (s, 2H, triazole); ¹³C NMR (CD₃OD, 100 MHz): 11.6, 13.7, 22.9, 24.0, 26.3, 27.6, 27.8, 29.0, 31.4, 37.5, 39.1, 42.4, 44.1, 44.7, 47.8, 54.5, 114.4, 125.6, 127.5, 131.0, 134.4, 137.8 and 152.2.

Microanalysis: C, 75.70 (expected 75.95); H, 8.79 (expected 8.76); N, 10.70 (expected 11.07).

### 2-Ethyl-3-O-sulfamoyl-17β-(2-[1,2,4]triazol-4-yl-ethyl)-estra-[1,3,5]-triene 51

An ice cold solution of sulfamoyl chloride (0.8 mmol) in DMA (1 mL) was treated with 50 (80 mg, 0.21 mmol) then stirred 16 h at room temperature. After addition of water (5 mL) the mixture was extracted with ethyl acetate (2×50 mL), the combined organic layers were then washed with water, brine, dried and evaporated. The resultant solid was purified by flash chromatography (hexane/ethyl acetate 1:1 to 1:3) to a white powder. White powder, 65 mg (68%), mp=245-246° C.; ¹H NMR (CD₃OD/DMSO-d6 10:1, 270 MHz): 0.70 (s, 3H, CH₃), 1.18 (t, J=7.3 Hz, 2H, CH₃), 1.20-1.58 (m, 8H), 1.65-2.10 (m, 6H), 2.21-2.32 (m, 1H), 2.32-2.43 (m, 1H), 2.72 (q, J=7.3 Hz, 2H, CH₂), 2.84 (m, 2H, H6), 4.09-4.23 (m, 2H, H2′), 7.06 (s, 1H, ArH), 7.21 (s, 1H, ArH), 8.61 (s, 2H, triazole). ¹³C NMR (CD₃OD/DMSO-d6 10:1, 100 MHz): 11.7, 14.0, 22.7, 24.1, 26.1, 27.5, 27.6, 29.0, 31.5, 37.4, 39.1, 42.3, 44.3, 44.6, 47.9, 54.5, 121.6, 126.4, 133.9, 135.4, 138.7 and 146.6.

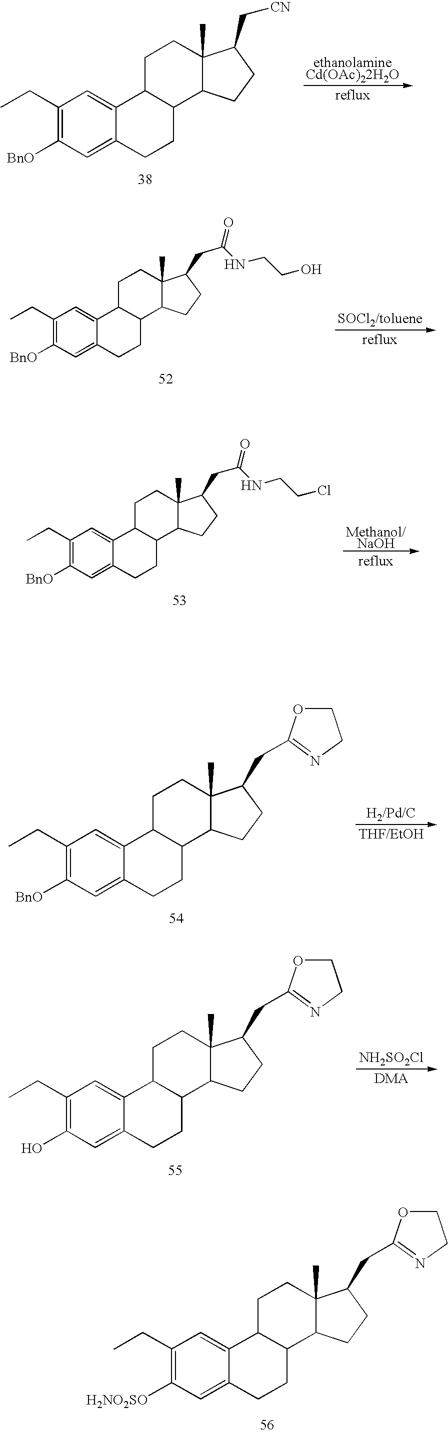

### 2-Ethyl-3-O-benzyl-17β-(N-(2-hydroxyethyl))-acetamido) 17-deoxyestrone 52

A solution of 38 (620 mg, 1.5 mmol), Cd(OAc)₂.2H₂O (20 mg, 0.075 mmol) in ethanolamine (3 ml) was refluxed for 24 hours. After cooling the mixture to room temperature, 50 ml water was added and the organics were extracted with ethyl acetate (2×50 ml). The organic layer washed with water, brine, dried over magnesium sulfate and the solvent removed under reduced pressure. The residual oil was purified by flash chromatography (Hexane/ethyl acetate 3:1 to 0:1) to give 52 as a white solid, 520 mg (73%), mp=189-190° C.; ¹H NMR (CDCl₃, 270 MHz): 0.62 (s, 3H, CH₃), 1.19 (t, J=7.4 Hz, 2H, CH₃), 1.21-1.54 (m, 7H), 1.73-2.04 (m, 5H), 2.17-2.37 (m, 3H), 2.65 (q, J=7.4 Hz, 2H, CH₂), 2.80 (m, 3H, H6+H1′), 3.41 (m, 2H, CH₂N), 3.71 (m, 2H, CH₂O), 5.02 (s, 2H, CH₂Ph), 5.99(m, 1H, NH), 6.61 (s, 1H, ArH), 7.08 (s, 1H, ArH), 7.26-7.45 (m, 5H, 5H). ¹³C NMR (CDCl₃, 100 MHz): 12.7, 14.7, 23.5, 24.3, 26.5, 27.9, 28.3, 29.8, 37.5, 37.7, 39.0, 42.6, 44.1, 47.6, 54.3, 62.7, 69.8, 111.9, 126.3, 127.1, 127.7, 128.5, 130.2, 132.4, 135.1, 137.8 and 154.5 and 174.6. LRMS(M+1)⁺: 476.33 (expected 476.32).

### 2-Ethyl-3-O-benzyl-17β-(N-(2-chloro-ethyl))-acetamido) 17-deoxyestrone 53

A solution of 52 (475 mg, 1 mmol) and thionyl chloride (0.28 ml, 4 mmol) in 25 ml toluene was refluxed for 1 hour and after cooling the solution to room temperature the solvent was evaporated under reduced pressure. The residual oil was dissolved in ethyl acetate (80 ml), the organic layer washed with water, brine, dried over magnesium sulfate and the solvent was removed under reduced pressure. The residual oil was purified by flash chromatography (hexane/ethyl acetate 5:1 to 2:1) to give 53 as a beige solid, 430 mg (87%), mp=112-113° C.; ¹H NMR (CDCl₃, 270 MHz): 0.63 (s, 3H, CH₃), 1.20 (t, J=7.4 Hz, 2H, CH₃), 1.23-1.54 (m, 7H), 1.60-2.06 (m, 6H), 2.18-2.37 (m, 3H), 2.62 (q, J=7.4 Hz, 2H, CH₂), 2.80 (m, 2H, H6), 3.55-3.64 (m, 4H, NCH₂CH₂Cl), 5.02 (s, 2H, CH₂Ph), 5.90(m, 1H, NH), 6.62 (s, 1H, ArH), 7.09 (s, 1H, ArH), 7.26-7.45 (m, 5H, 5H). ¹³C NMR (CDCl₃, 100 MHz): 12.7, 14.7, 23.5, 24.3, 26.5, 27.9, 28.3, 29.8, 37.5, 37.7, 39.0, 41.2, 42.6, 44.1, 44.3, 47.5, 54.3, 62.7, 69.9, 111.9, 126.3, 127.1, 127.7, 128.5, 130.2, 132.5, 135.1, 137.8 and 154.5 and 173.4. LRMS(M+1)⁺: 476.33 (expected 476.32)

### 2-Ethyl-3-O-benzyl-17β-(oxazolin-2-yl-methyl) 17-deoxyestrone 54

A solution of 53 (395 mg, 0.8 mmol) and sodium hydroxide (80 mg, 2 mmol) in 20 ml methanol was refluxed for 3 hours. The solvent as evaporated under reduce pressure and the residual solid was poured in water (50 ml) and ethyl acetate (80 ml). The organic layer washed with water, brine, dried over magnesium sulfate and the solvent removed under reduced pressure. The residual oil was purified by flash chromatography (hexane/ethyl acetate 5:1 to 3:1) to give 54 as a white solid, 270 mg (73%), mp=152-153° C.; ¹H NMR (CDCl₃, 270 MHz): 0.65 (s, 3H, CH₃), 1.20 (t, J=7.3 Hz, 2H, CH₃), 1.23-1.56 (m, 7H), 1.67-2.02 (m, 5H), 2.10-2.43 (m, 5H), 2.65 (q, J=7.3 Hz, 2H, CH₂), 2.80 (m, 2H, H6), 3.80 (t, J=9.4 Hz, 2H, CH₂N), 4.20 (t, J=9.4 Hz, 2H, CH₂O), 5.02 (s, 2H, CH₂Ph), 5.90(m, 1H, NH), 6.62 (s, 1H, ArH), 7.09 (s, 1H, ArH), 7.28-7.45 (m, 5H, 5H).

### 2-Methoxy-3-O-benzyl-17-O-(N-trichloroacetyl)-carbamoyl-estradiol 55

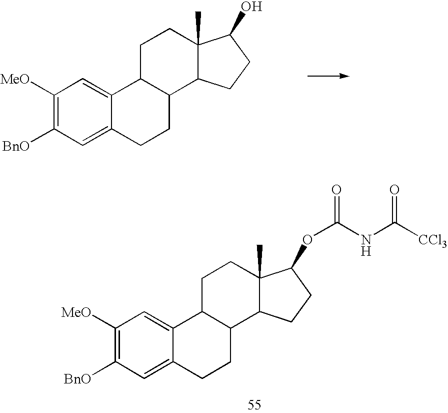

Trichloroacetylisocyanate (0.20 ml, 316 mg, 1.68 mmol) was added to a solution of 2-methoxy-3-O-benzyl-estradiol (393 mg, 1.00 mmol) in THF (20 ml). The solution was stirred for 15 min at r.t. and water (0.5 ml) was added to destroy the excess of trichloroacetylisocyanat. Then EtOAc (50 ml) and more water (30 ml) were added, the organic layer was separated, dried over Na₂SO₄ and concentrated under reduced pressure. The residue was purified by column chromatography (EtOAc/hexane 1:5, R_{f}: 0.34) to give the product as a white solid. Yield: 534 mg (92%). m.p. 193-195° C.; IR (CH₂Cl₂-solution): ν=3520, 3390, 2985, 1805 (C═O), 1746 (C═O), 1490 cm⁻¹; ¹H NMR (400 MHz, CDCl₃) δ 0.91 (s, 3H, 18−H), 1.26-1.28 (m, 6H), 1.65-1.91 (m, 3H), 1.95-2.02 (m, 1H), 2.20-2.40 (m, 3H), 2.68-2.84 (m, 2H), 3.88 (s, 3H, —OCH₃), 4.82 (dd, J=9.0, 7.8 Hz, 1H, H-17), 5.11 (s, 2H, —CH₂Ph), 6.63 (s, 1H), 6.84 (s, 1H), 7.27-7.47 (m, 5H), 8.31 (s, 1H, —NH); ¹³C NMR (100.5 MHz, CDCl₃) δ 12.32, 23.24, 26.37, 27.31, 27.53, 29.17, 36.83, 38.49, 43.18, 44.06, 49.53, 56.32, 71.04, 86.27, 91.79, 109.58, 114.42, 127.12, 127.56, 128.33, 128.50, 132.30, 137.20, 146.16, 147.38, 149.64, 157.38; MS (FAB+): m/z 73 (58%), 375.2 (66%), 579.1 (100%, [C₂₉H₃₂Cl₃NO₅]⁺); HRMS (FAB+) calcd for C₂₉H₃₂Cl₃NO₅: 579.1346; Found, 579.1323. Anal. calcd (%) for C₂₉H₃₂Cl₃NO₅ (580.9): C, 59.96; H, 5.55; N, 2.41; Found: C, 59.7; H, 5.54; N, 2.40.

### 2-Methyoxy-3-O-benzyl-estradiol-17-carbamate 56

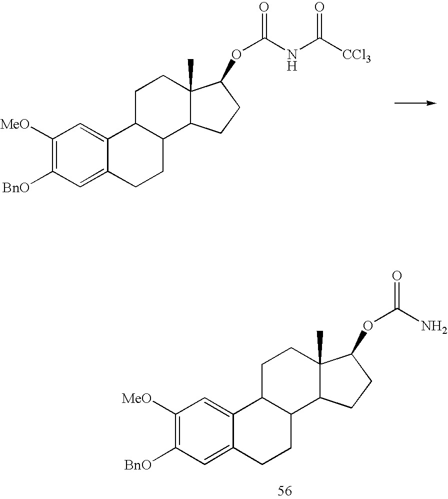

A solution of K₂CO₃ (414 mg, 3.0 mmol) in water (10 ml) was added to a solution of 55 (1.102 g, 2.0 mmol) in THF (20 ml) and MeOH (20 ml). The mixture was stirred for 3 h at r.t. (TLC-control), EtOAc (60 ml) and water (60 ml) were added, the organic layer was separated, dried over Na₂SO₄ and concentrated under reduced pressure. The residue was crystallised from DCM/hexane. Yield: 745 mg (92%) colourless needles. m.p. 180-181° C.; IR (CH₂Cl₂-solution): ν=3536, 3423, 2935, 1726 (C═O), 1584, 1513 cm⁻¹; ¹H-NMR (400 MHz, CDCl₃) δ 0.82 (s, 3H, H-18), 1.25-1.64 (m, 7H), 1.68-1.78 (m, 1H), 1.82-1.96 (m, 2H) 2.16-2.32 (m, 3H), 2.64-2.81 (m, 2H), 3.87 (s, 3H, —OCH₃), 4.58 (bs, 2H, —NH₂) 4.63 (dd, J=9.4, 8.2 Hz, 1H, H-17), 5.11 (s, 2H, —CH₂Ph), 6.62 (s, 1H), 6.84 (s, 1H), 7.27-7.46 (m, 5H); MS (FAB+): m/z 90.9 (100% [C₇H₇]⁺), 435.0 (90%, [C₂₇H₃₃NO₄]⁺); HRMS (FAB+) calcd for C₂₇H₃₃NO₄: 435.2410; Found 435.2404.

### 2-Methoxy-estradiol-17-carbamate 57

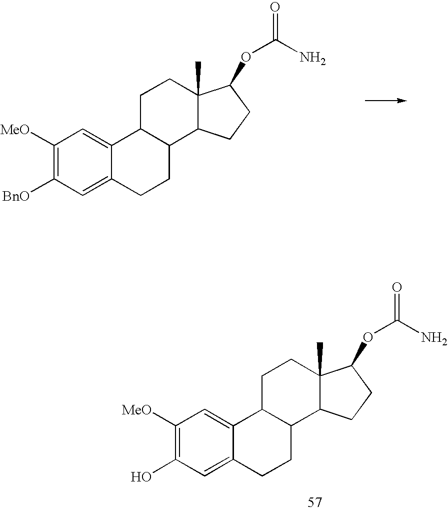

Palladium on charcoal (50 mg, 10%) was added to a solution of 3-O-benzyl-estradiol-17-carbamate (405 mg, 1.0 mmol) in MeoH (10 ml) and THF 10 ml). The mixture was stirred under H₂-atmosphere for 18 h (balloon), filtered through a layer of celite (ca. 3 cm) and concentrated under reduced pressure. The residue was crystallised from EtOAc/hexane. Yield: 271 mg (86%) fine white needles.

m.p. 235-238° C.; IR (CH₂Cl₂-solution): ν=3685, 3537, 3424, 3049, 2936, 1726 (C═O), 1584, 1506, 1344, 1068 cm⁻¹; ¹H-NMR (400 MHz, DMSO-d₆) δ 0.77 (s, 3H, H-18), 1.28-1.50 (m, 7H), 1.59-1.70 (m, 1H), 1.72-1.81 (m, 2H), 1.99-2.16 (m, 2H), 2.22-2.31 (m, 1H), 2.55-2.68 (m, 2H), 3.70 (s, 3H, —OCH₃), 4.45 (dd, J=9.0, 7.8 Hz, 1H, H-17), 6.40 (bs, 2H, —NH), 6.43 (s, 1H), 6.75 (s, 1H), 8.60 (s, 1H, —OH); MS (FAB+): m/z 345.2 (100%, [C₂₀H₂₇NO₄]⁺); HRMS (FAB+) calcd for C₂₀H₂₇NO₄: 345.1940; Found 345.1943. Elemental analysis calcd (%) for C₂₀H₂₇NO₄ (345.4): C, 69.54; H, 7.88; N, 4.05; found: C 69.4, H 7.81, N 3.95.

### 2-Methoxy-3-O-sulfamoyl estradiol-17-carbamate 58

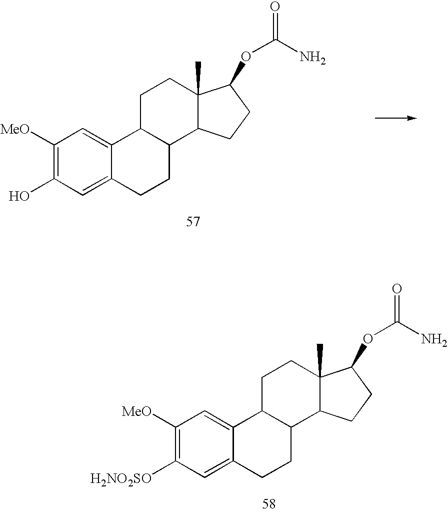

Sulfamoyl chloride solution in toluene (3 ml, 0.7 M, 2.1 mmol) was concentrated under reduced pressure to ca. 0.5 ml volume. The residue was cooled to 0° C. (icebath) and DMA (5 ml) was added slowly. 13 (120 mg, 0.35 mmol) was added to the colourless solution and the mixture was stirred for 18 h at r.t. EtOAc (50 ml) and water (50 ml) were added, the organic layer was separated, washed with water (2×30 ml) and brine (20 ml), dried over Na₂SO₄ and concentrated under reduced pressure. The residue was crystallised from acetone/cyclohexane to give monoclinic crystals. Yield 130 mg (88%). m.p. 201-204° C.; IR (CH₂Cl₂-solution): ν=3686, 3536, 3423, 3326, 3063-2880, 1727 (C═O), 1584, 1506, 1398, 1190, 1112, 1070 cm⁻¹; ¹H-NMR (400 MHz, DMSO-d₆) δ 0.78 (s, 3H, H-18), 1.30-1.52 (m, 7H), 1.61-1.70 (m, 1H), 1.76-1.84 (m, 2H), 2.00-2.12 (m, 1H), 2.16-2.24 (m, 1H), 2.33-2.40 (m, 1H), 2.70-2.76 (m, 2H), 3.76 (s, 3H, —OCH₃), 4.47 (dd, J=9.0, 7.8 Hz, 1H, H-17), 6.40 (bs, 2H, —NH₂), 6.98 (s, 2H, 2×Ar—H), 7.83 (s, 2H, —NH₂); MS (FAB+): m/z 424.1 (100%, [C₂₀H₂₈N₂O₆S]⁺); HRMS (FAB+) calcd for C₂₀H₂₈N₂O₆S: 424.166809; found, 424.166595.

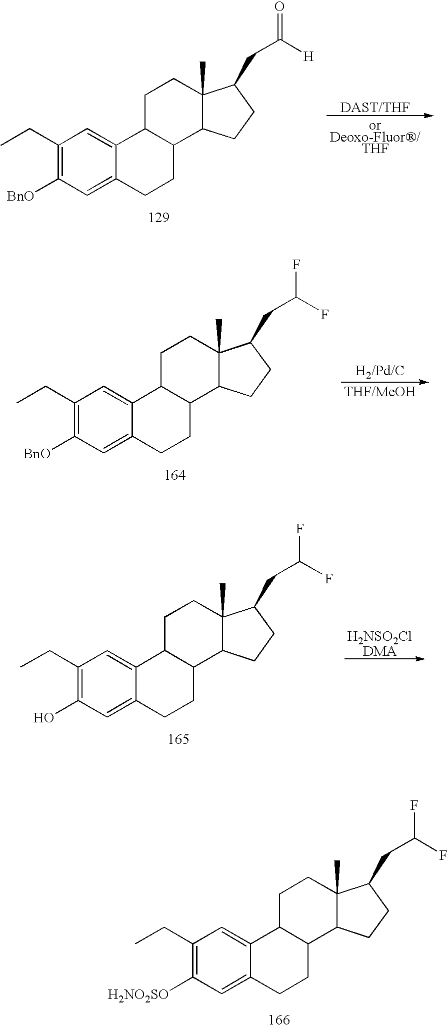

### 2-Ethyl-3-O-benzyl-17β-(2,2-difluoro-ethyl)-estra-[1,3,5]-triene 164

A solution of 129 (430 mg, 1 mmol) in dry THF (10 mL) was cooled to 0° C. before DAST (0.37 mL, 3 mmol) or Deoxo-Fluor® (0.55 mL, 3 mmol) was added dropwise. The mixture was stirred at 0° C. under nitrogen for 2 hours (very slow reaction) then 1 day at room temperature. After drop wise addition of a saturated solution of NaHCO₃ (5 mL), the organics were extracted with ethyl acetate (80 mL) and the organic layer washed successively with water and brine, dried (MgSO₄) and the solvents removed under reduced pressure. The crude oil was purified by flash chromatography (hexane/ethyl acetate 100:1) to afford 210 mg of 2-ethyl-3-O-benzyl-17β-(2,2-difluoro-ethyl)-estra-[1,3,5]-triene 164 (48%) m.p. 114-115° C. R_{f}: 0.51 (ethyl acetate/hexane, 1:20). ¹H NMR (270 MHz, CDCl₃) δ 0.64 (3H, s, CH₃), 1.22 (3H, t, J=7.3 Hz, CH₂CH₃), 1.26-2.09 (14H, m), 2.20-2.29 (1H, m), 2.31-2.40 (1H, m), 2.68 (2H, q, J=7.3 Hz, CH₂CH₃), 2.83 (2H, m, H6), 5.05 (2H, s, OCH₂Ph), 5.84 (1H, tdd, J_{HF}=57.3, J_{HH}=5.0 and 3.7 Hz, CHF₂), 6.64 (1H, s, ArH), 7.12 (1H, s, ArH), 7.29-7.46 (5H, m, Ph); ¹³C NMR (100 MHz, CDCl₃) δ 12.6 (CH₃), 14.7 (CH₃), 23.5, 24.5, 26.5, 28.0, 28.3, 29.8, 35.1 (t, ²J_{CF}=20 Hz, CH₂^{1′}), 37.5, 38.9, 42.6, 44.2, 44.6 (t, ³J_{CF}=4.6 Hz, C17), 54.3, 69.8 (OCH₂Ph), 111.9, 117.8(t, ¹J_{CF}=239 Hz, CHF₂) 126.3, 127.1, 127.7, 128.5, 130.3, 132.4, 135.1, 137.8 and 154.5.

### 2-Ethyl-3-hydroxy-17β-(2,2-difluoro-ethyl)-estra-[1,3,5]-triene 165

A solution of 164 (230 mg, 0.54 mmol) in THF (3 mL) and methanol (15 mL) was stirred with 40 mg of 5% Pd/C under hydrogen for 16 hours. After filtration over celite and washing with ethyl acetate, the solvents were evaporated under reduced pressure. The crude oil was purified by flash chromatography (Hexane/ethyl acetate 50:1 to 40:1) to give a light orange coloured solid (160 mg, 86%) which slowly crystallizes under vacuum. R_{f}: 0.15 (ethyl acetate/hexane 1:20) and 0.48 (ethyl acetate/hexane 1:3). ¹H NMR (270 MHz, CDCl₃) δ 0.62 (3H, s, CH₃), 1.21 (3H, t, J=7.3 Hz, CH₂CH₃), 1.23-2.07 (14H, m), 2.16-2.24 (1H, m), 2.27-2.36 (1H, m), 2.59 (2H, q, J=7.3 Hz, CH₂CH₃), 2.78 (2H, m, H6), 4.58 (1H, s, OH), 5.84 (1H, tdd, J_{HF}=57.2, J_{HH}=9.2 and 5.2 Hz, CHF₂), 6.49 (1H, s, ArH), 7.05 (1H, s, ArH), ¹³C NMR (100 MHz, CD₃COCD₃) δ 12.6 (CH₃), 14.5 (CH₃), 23.1, 24.5, 26.5, 27.9, 28.3, 29.3, 35.1 (t, ²J_{CF}=20 Hz, CH₂^{1′}), 37.4, 38.9, 42.6, 44.1, 44.5 (t, ³J_{CF}=4.6 Hz, C17), 54.3, 115.2, 117.8 (t, ³J_{CF}=239 Hz, CHF₂) 120.2, 126.3, 127.2, 132.7, 135.6 and 151.2. LC/MS (APCI−) tᵣ=1.22 min m/z 347.41 (M⁺+H). (MeOH/H₂O 95/5). HPLC tᵣ=5.12 min (99.6) (MeOH/H₂P 90/10)

### 2-Ethyl-3-O-sulfamoyl-17β-(2,2-difluoro-ethyl)-estra-[1,3,5]-triene 166

A solution of 165 (92 mg, 0.26 mmol) and sulfamoyl chloride (0.52 mmol) in DMA (1 mL) was stirred at room temperature under nitrogen for 24 hours. After addition of water (5 mL), the organics were extracted with ethyl acetate and the organic layer washed with water, brine, dried (MgSO₄) and evaporated. The product was purified by flash chromatography (hexane/ethyl acetate 10:1) to give 2-Ethyl-3-O-sulfamoyl-17β-(2,2-difluoro-ethyl)-estra-[1,3,5]-triene 166 (95 mg, 86%) as a white solid that was recrystallized in ethyl acetate and hexane 1:20 (80 mg, 72%). mp 164-165° C. R_{f}: 0.30 (hexane/ethyl acetate 3:1). ¹H NMR (270 MHz, CDCl₃) δ 0.62 (3H, s, CH₃), 1.20 (3H, t, J=7.3 Hz, CH₂CH₃), 1.23-2.08 (17H, m), 2.18-2.35 (2H, m), 2.68 (2H, q, J=7.3 Hz, CH₂CH₃), 2.82 (2H, m, H6), 4.93 (2H, br, NH₂), 5.82 (1H, tt, J_{HF}=57 Hz, J_{HH}=4.7 Hz, CHF₂), 7.06 (1H, s, ArH), 7.18 (1H, s, ArH). ¹³C NMR (100 MHz, CDCl₃) δ 12.6 (CH₃), 14.7 (CH₃), 23.1, 24.5, 26.2, 27.6, 28.3, 29.2, 35.1 (t, ²J_{CF}=20 Hz, CH₂^{1′}), 37.3, 38.4, 42.5, 44.3, 44.5 (t, ³J_{CF}=3.8 Hz, C17), 54.3, 111.9, 117.7 (t, ¹J_{CF}=239 Hz, CHF₂) 121.4, 127.0, 133.6, 136.1, 139.6 and 146.1; LC/MS (APCI−) tᵣ=1.53 min m/z 426.19 (M⁺−H). (MeOH/H₂O 95/5); HPLC tᵣ=3.77 min (MeOH/H₂O 90/10).

### 2-Ethyl-3-O-sulfamoyl-17β-(methanesulfonyl)amino estra-[1,3,5]-triene 43b and 2-methoxy-3-O-sulfamoyl-17β-(methanesulfonyl)amino estra-[1,3,5]-triene 43c

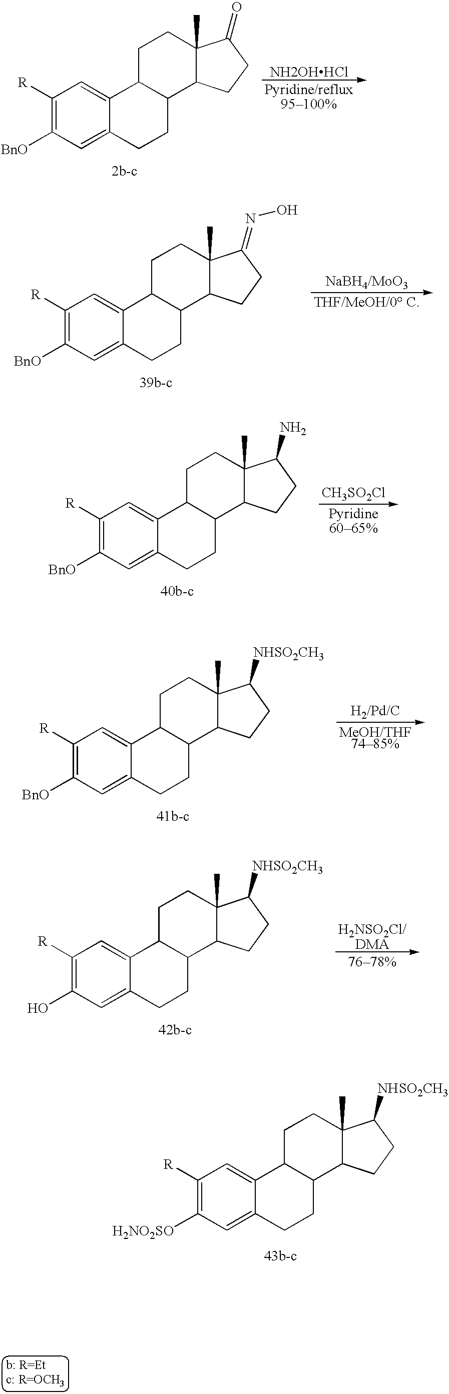

### Synthesis of 2-ethyl-3-O-benzyl-estrone oxime 39b and 2-methoxy-3-O-benzyl-estrone oxime 39c

A solution of the appropriate benzyl protected estrone (4 mmol) and hydroxylamine hydrochloride (8 mmol) in pyridine (5 mL) was refluxed for 2-4 hours then cooled to room temperature. After addition of water (50 mL) the reaction was extracted with ethyl acetate (2×50 mL) and the combined organic layers were washed with water, brine, dried (MgSO4), filtered and the solvents evaporated under reduced pressure. The desired product were then recrystallised from hexane/ethyl acetate 5:1 (39b) 3:1 (39c). 2-ethyl-3-O-benzyl-estrone oxime 39b. White solid, Mp=96-98° C., 1.58 g (97%). Rf=0.21 (hexane/ethyl acetate 3:1) ¹H NMR (270 MHz, CDCl₃): 0.96 (3H, s, CH₃), 1.21 (3H, t, J=7.3 Hz, CH₃), 1.34-1.72 (6H, m), 1.92-1.98 (2H, m), 2.05-2.09 (1H, m), 2.25-2.47 (2H, m), 2.54-2.60 (2H, m), 2.66 (2H, q, J=7.3 Hz, CH₂), 2.77 (2H, m, H6), 5.04 (2H, s, CH₂Ph), 6.64 (1H, s, ArH), 7.11 (1H, s, ArH), 7.29-7.47 (5H, m, Ph), 8.21 (1H, s, OH). ¹³C NMR (100 MHz, CDCl₃): 14.6(CH₃), 17.2, 22.9, 23.4, 25.1, 26.2, 27.3, 29.6, 34.1, 38.2, 44.1, 44.3, 52.8, 69.8, 111.9, 126.2, 127.1, 127.6, 128.4, 130.4, 131.9, 134.8, 137.7, 154.5 and 171.2 (C17).

2-methoxy-3-O-benzyl-estrone oxime 39c. White solid, Mp=157-158° C., 1.56 g (96%). Rf=0.12 (hexane/ethyl acetate 3:1) ¹H NMR (270 MHz, CDCl₃): 0.95 (3H, s, CH₃), 1.29-1.70 (6H, m), 1.90 (2H, m), 2.01-2.08 (1H, m), 2.23-2.37 (2H, m), 2.52-2.61 (2H, m), 2.70-2.85 (2H, m, H6), 3.86 (3H, s, CH₃O), 5.10 (2H, s, CH₂Ph), 6.62 (1H, s, ArH), 6.83 (1H, s, ArH), 7.25-7.45 (m, 5H, Ph), 8.10 (1H, s, OH). ¹³C NMR (270 MHz, CDCl₃): 17.3 (CH₃), 22.9, 25.1, 26.4, 27.3, 29.1, 34.1, 38.1, 44.3, 44.4, 52.9, 56.3(CH₃O), 71.1 (CH₂Ph), 109.6, 114.6, 127.3, 127.8, 128.5 128.7, 132.6, 137.4, 146.4, 147.6 and 171.3 (C17).

### 2-ethyl-3-O-benzyl-17β-amino estra-[1,3,5]-triene 40b and 2-methoxy-3-O-benzyl-17β-3-amino estra-[1,3,5]-triene 40c

A solution of the appropriate oxime (39b or 39c) (2 mmol) in THF (5 mL) and methanol (20 mL) was cooled to 0° C. before adding MoO₃ (4.4 mmol, 0.63 g) then NaBH₄ (4.4 mmol, 0.17 g) in a portion wise manner. The suspension was stirred at 0° C. for 8-10 h then treated with 1M aqueous potassium hydroxide (5 mL). The suspension was then stirred at room temperature for 16 h, then cooled to 0° C. and filtered through celite and the salts washed with methanol. The filtrate was concentrated under reduced pressure and the residual oil dissolved in ethyl acetate then washed with water, brine, dried (MgSO₄) and evaporated. The crude oil was purified by flash chromatography (ethyl acetate/methanol/TEA 20:1:0.2).

2-ethyl-3-O-benzyl-17β-amino estra-[1,3,5]-triene 40b White solid, Mp=103-105° C., 570 mg (73%). R_{f}: 0.25 (ethyl acetate/methanol/Et₃N, 10:1:0.2), ¹H NMR (270 MHz, CDCl₃) δ 0.66 (3H, s, CH₃), 1.20 (3H, t, J=7.3 Hz, CH₂CH₃), 1.22-1.56 (8H, m), 1.65-1.74 (1H, m), 1.81-1.92 (2H, m), 1.97-2.25 (2H, m), 2.30-2.40 (1H, m), 2.66 (2H, q, J=7.3 Hz, CH₂CH₃), 2.70-2.86 (4H, m, H6+NH₂), 3.34 (1H, m, H17), 5.03 (2H, s, CH₂Ph), 6.62 (1H, s, ArH), 7.10 (1H, s, ArH), 7.28-7.45 (5H, m, Ph). ¹³C NMR (100 MHz, CDCl₃) δ 11.2 (CH₃), 18.7, 23.4, 24.3, 26.4, 27.6, 29.7, 31.0, 37.7, 44.1, 45.3, 52.7, 54.7 (CH₃O), 63.1(C17), 70.4 (CH₂Ph), 111.9, 126.3, 127.1, 127.6, 128.5 130.2, 132.5, 135.1, 137.8, and 154.5 (C17).

2-methoxy-3-O-benzyl-17β-amino estra-[1,3,5]-triene 40c, white solid, Mp=90-92° C., 1.45 g (85%), R_{f}: 0.18 (ethyl acetate/methanol/Et₃N, 10:1:0.2) ¹H NMR (270 MHz, CDCl₃): 0.67 (3H, s, CH₃), 1.20-1.58 (9H, m), 1.65-1.74 (1H, m), 1.82-1.89 (2H, m), 2.00-2.32 (2H, m), 2.67-2.82 (3H, m, H6+H1′), 3.80 (3H, s, CH₃O), 5.09 (2H, s, CH₂Ph), 6.61 (1H, s, ArH), 6.84 (1H, s, ArH), 7.26-7.44 (m, 5H, Ph). ¹³C NMR (270 MHz, CDCl₃): 11.2 (CH₃), 23.4, 26.6, 27.6, 29.2, 31.5, 36.8, 39.1, 43.0, 44.4, 52.1, 56.3 (CH₃O), 63.0 (C17), 71.1 (CH₂Ph), 109.7, 114.6, 127.3, 127.7, 128.5 128.9, 133.1, 137.5, 146.3 and 147.5.

### 2-Ethyl-3-O-benzyl-17β-(methanesulfonyl)amino estra-[1,3,5]-triene 41b and 2-methoxy-3-O-benzyl-17β-(methanesulfonyl)amino estra-[1,3,5]-triene 41c

A solution of 40b-c (1.5 mmol) in dry pyridine (5 mL) was cooled to 0° C. and then treated with methane sulfanyl chloride (1.8 mmol, 0.14 mL) in a dropwise manner. The solution was stirred at 0° C. for 4 h then at room temperature for 6 h before adding water (10 mL). The reaction was then extracted with ethyl acetate (2×50 mL), the combined organic layers washed with water (3×50 mL), brine (100 mL, 4×50 mL), dried (MgSO₄), and evaporated to give a light yellow oil which was purified by flash chromatography (silica: eluent:ethyl acetate/hexane, 1:3 to 1:1).

2-Ethyl-3-O-benzyl-17β-(methanesulfonyl)amino estra-[1,3,5]-triene 41b white solid, mp 188-189° C., (420 mg, 60%). R_{f}: 0.15 (ethyl acetate/hexane, 1:3), 0.30 (ethyl acetate/hexane 1:2). ¹H NMR (270 MHz, CDCl₃) δ 0.73 (3H, s, CH₃), 1.20 (3H, t, J=7.3 Hz, CH₂CH₃), 1.24-1.56 (7H, m), 1.73-1.98 (3H, m), 2.17-2.28 (2H, m), 2.33-2.41 (1H, m), 2.66 (2H, q, J=7.3 Hz, CH₂CH₃), 2.82 (2H, m, H6), 2.98 (3H, s, CH₃SO₂), 3.34 (1H, m, H17), 4.20 (1H, d, J=9.6 Hz, NH), 5.04 (2H, s, CH₂Ph), 6.63 (1H, s, ArH), 7.10 (1H, s, ArH), 7.28-7.46 (5H, m, Ph); ¹³C NMR (100 MHz, CDCl₃) 111.9 (CH₃), 14.7 (CH₃), 23.2, 23.5, 26.2, 27.4, 29.7, 30.2, 36.7, 39.0, 41.7(CH₃SO₂), 42.9, 43.9, 51.2, 63.6 (C17), 69.8 (CH₂PH), 111.9, 126.3, 127.1, 127.7, 128.5, 130.4, 131.9, 134.9, 137.7 and 154.6. LRMS (FAB+) m/z 467.3 (M⁺), 466.3 (100), 376.3 (M⁺−PhCH₂).

2-Methoxy-3-O-benzyl-17β-(methanesulfonyl)amino estra-[1,3,5]-triene 41c pale yellow solid, mp 199-200° C., (450 mg, 64%). R_{f}: 0.58 (ethyl acetate/hexane, 1:1). ¹H NMR (270 MHz, CDCl₃) δ 0.73 (3H, s, CH₃), 1.22-1.58 (8H, m), 1.72-1.87 (2H, m), 1.93-1.99 (1H, m), 2.16-2.33 (3H, m), 2.72 (2H, m, H6), 2.97 (3H, s, CH₃SO₂), 3.34 (1H, q, =9.1 Hz, H17), 3.84 (3H, s, CH₃O), 4.27 (1H, d, J=9.1 Hz, NH), 5.09 (2H, s, CH₂Ph), 6.61 (1H, s, ArH), 6.82 (1H, s, ArH), 7.26-7.45 (5H, m, Ph); ¹³C NMR (100 MHz, CDCl₃) δ 11.9 (CH₃), 23.2, 26.3, 27.3, 29.1, 30.1, 36.7, 38.8, 41.7, 42.9, 44.2, 51.2, 56.3 (CH₃O), 63.5, 71.1 (CH₂Ph), 109.7, 114.6, 127.3, 127.8, 128.5 128.7, 132.6, 137.4, 146.4 and 147.6. LRMS (FAB−) m/z 467.3 (M⁺,), 469.3 (M⁺), 468.3 (M⁺−1, 100%), 376.3 (M⁺−NSO₂CH₃).

### 2-Ethyl-3-hydroxy-17β-(methanesulfonyl)amino estra-[1,3,5]-triene 42b and 2-methoxy-3-hydroxy-17β-(methanesulfonyl)amino estra-[1,3,5]-triene 42c

A mixture of 41b-c (1 mmol) and 5% Pd/C (50 mg) in THF (5 mL) and methanol (20 mL) was stirred at room temperature under an atmosphere of hydrogen for 24 h. After filtration over ceilte/sand the solvents were evaporated and the residual solid was purified by flash chromatography (hexane/ethyl acetate 3:1 to 3:2) and the solid obtained after evaporation of the solvents under reduced pressure was recrystallized (hexane/ethyl acetate 3:2).

2-Ethyl-3-hydroxy-17β-(methanesulfonyl)amino estra-[1,3,5]-triene 42b, white solid, mp 267-268° C., 280 mg (74%). mp 267-268° C. R_{f}: 0.20 (ethyl acetate/hexane, 1:2). ¹H NMR (270 MHz, CD₃COCD₃) δ 0.71 (3H, s, CH₃), 1.07 (3H, t, J=7.3 Hz, CH₂CH₃), 1.17-1.41 (7H, m), 1.52-1.80 (3H, m), 1.85-1.91 (1H, m), 2.06-2.20 (2H, m), 2.23-2.31 (1H, m), 2.50 (2H, q, J=7.3 Hz, CH₂CH₃), 2.62-2.68 (2H, m, H6), 2.84 (3H, s, CH₃SO₂), 3.25 (1H, m, H17), 5.73 (1H, d, J=9.2 Hz, NH), 6.42 (1H, s, ArH), 6.93 (1H, s, ArH), 7.70 (1H, s, OH); ¹³C NMR (100 MHz, CD₃COCD₃) δ 11.4 (CH₃), 14.2 (CH₃), 23.0, 23.1, 26.2, 27.4, 28.4, 29.1, 36.8, 39.3, 40.4 (CH₃SO₂), 42.8, 44.1, 51.2, 63.6 (C17), 114.9, 126.2, 127.5, 130.9, 134.6, and 152.6. LRMS (FAB+) m/z 377.3 (M⁺), 376.4 (M⁺−1, 100)

2-Methoxy-3-hydroxy-17β-(methanesulfonyl)amino estra-[1,3,5]-triene 42c white solid, mp 199-200° C., 320 mg, (85%). R_{f}: 0.33 (ethyl acetate/hexane, 1:1). ¹H NMR (270 MHz, CDCl₃) δ 0.74 (3H, s, CH₃), 1.18-1.55 (7H, m), 1.72-1.87 (2H, m), 1.92-1.97 (1H, m), 2.16-2.32 (3H, m), 2.75 (2H, m, H6), 2.97 (3H, s, CH₃SO₂), 3.33 (1H, q, =9.2 Hz, H17), 3.85 (3H, s, CH₃O), 4.34 (1H, d, J=9.2 Hz, NH), 5.45 (1H, s, OH), 6.63 (1H, s, ArH), 6.76 (1H, s, ArH); ¹³C NMR (100 MHz, CDCl₃) δ 11.9 (CH₃), 23.3, 26.4, 27.3, 29.0, 30.3, 36.7, 38.8, 41.8, 42.9, 44.3, 51.3, 56.2 (CH₃O), 63.7, 108.1, 114.6, 129.5, 131.6, 143.7 and 144.7; LRMS (FAB+) m/z 379.3 (M⁺), 378.3 (M⁺−1, 100).

### 2-Ethyl-3-O-sulfamoyl-17β-(methanesulfonyl)amino estra-[1,3,5]-triene 43b and 2-methoxy-3-O-sulfamoyl-17β-(methanesulfonyl)amino estra-[1,3,5]-triene 43c

A solution of 42b-c (1 mmol) and sulfamoyl chloride (2 mmol) was stirred in DMA (1 mL) at room temperature under nitrogen for 14 hours. After addition of 5 mL of water, the organics were extracted with ethyl acetate (2×50 mL) and the organic layer was subsequently washed with water and brine, dried (MgSO₄) and the solvent evaporated under reduced pressure. The residual solid was purified by flash chromatography (hexane/ethyl acetate 3:1 to 1:1) and the solid obtained after evaporation of the solvent sunder reduced pressure was recrystallized (hexane/ethyl acetate 1:1).

2-Ethyl-3-O-sulfamoyl-17β-(methanesulfonyl)amino estra-[1,3,5]-triene 43b White needles, mp 240-241° C., 345 mg (76%). R_{f}: 0.64 (ethyl acetate/hexane 2:1). ¹H NMR (270 MHz, CDCl₃/CD₃OD 10:1) δ 0.71 (3H, s, CH₃), 1.19 (3H, t, J=7.3 Hz, CH₂CH₃), 1.22-1.51 (7H, m), 1.72-1.79 (1H, m), 1.83-1.88 (1H, m), 1.91-1.96 (1H, m), 2.12-2.22 (2H, m), 2.30-2.36 (1H, m), 2.67 (2H, q, J=7.3 Hz, CH₂CH₃), 2.80 (2H, m, H6), 2.95 (3H, s, CH₃SO₂), 3.30 (1H, t, J=9.2 Hz, H17), 7.07 (1H, s, ArH), 7.15 (1H, s, ArH). ¹³C NMR (100 MHz, CDCl₃/CD₃OD 10:1) δ 11.5 (CH₃), 14.4 (CH₃), 22.8, 23.0, 25.8, 26.9, 28.9, 29.4, 36.4, 38.4, 41.2 (CH₃SO₂), 42.6, 43.9, 51.0, 63.2 (C17), 121.4, 126.7, 133.7, 135.5, 138.6 and 146.2. LRMS (FAB+) m/z 377.3 (M⁺), 376.4 (M⁺−1, 100). Anal. Calcd. for C₂₁H₃₂N₂O₅S₂: C, 55.24; H, 7.06; N, 6.13. Found: C, 55.30; H, 7.10; N, 6.27%

2-Methoxy-3-O-sulfamoyl-17β-(methanesulfonyl)amino estra-[1,3,5]-triene 43c White needles, mp 198-199° C., 360 mg (78%). R_{f}: 0.40 (ethyl acetate/hexane, 2:1). ¹H NMR (270 MHz, CDCl₃/CD₃OD 10:1) δ 0.69 (3H, s, CH₃), 1.22-1.56 (9H, m), 1.69-2.04 (5H, m), 2.14-2.29 (3H, m), 2.76 (2H, m, H6), 2.94 (3H, S, CH₃SO₂), 3.30 (1H, q, =9.4 Hz, H17), 3.83 (3H, s, CH₃O), 4.72 (1H, d, J=9.1 Hz, NH), 6.87 (1H, s, ArH), 7.00 (1H, s, ArH). ¹³C NMR (100 MHz, CDCl₃) δ 13.1 (CH₃), 24.5, 27.4, 28.3, 30.1, 30.6, 31.1, 38.0, 39.8, 42.6, 43.2, 45.6, 52.7, 57.6 (CH₃O), 64.8, 11.7, 125.2, 131.0, 138.3, 141.2 and 150.8.

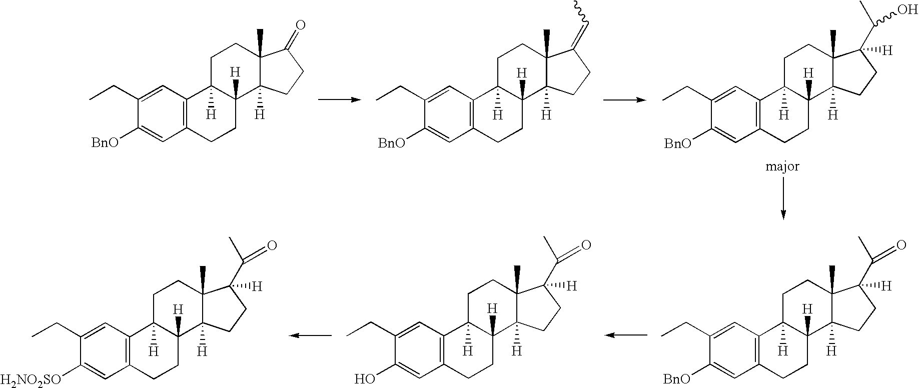

### 2-Ethyl-3-O-benzyl-17-(1-ethenyl) estra-[1,3,5]-triene

A solution of ethyl triphenylphosphonium iodide (2.5 g, 6.45 mmol) in DMSO (25 mL) was treated with sodium hydride (280 mg, 60% dispersion in mineral oil) and then brought to 100° C. for 0.25 h. 2-Ethyl-3-O-benzylestrone (1.23 g, 3.20 mmol) in DMSO was then added to the orange reaction mixture and heating was continued for a further 16 h. The cooled reaction mixture was then poured onto ice water (100 mL), extracted with ether (3×100 mL), the organics layers washed with water (3×100 mL), brine (10 mL) dried and evaporated. The crude product was purified by column chromatography (hexane/ethyl acetate gradient 100% to 97%) to give the desired alkene, 2-ethyl-3-O-benzyl-17-(1-ethenyl) estra-[1,3,5]-triene (760 mg, 59%), as a mixture of geometric isomers which showed significant resonances at δ_{H} 0.91 & 0.89 (3H, 2×s, 18-CH₃), 1.22 (3H, t, J 7.4, CH₂Me), 1.70 (app dt, J 7.2 & 1.7, :CHME major isomer), 2.68 (2H, q, J 7.4, CH₂Me), 2.74-2.90 (2H, m, 6-CH₂), 4.98-5.25 (1H, m, :CH both isomers), 5.40 (2H, s, OCH₂), 6.64 (1H, s, ArH), 7.12 (1H, s, ArH) and 7.27-7.48 (5H, m). C₂₉H₃₆O

### 2-Ethyl-3-O-benzyl-17β-(1-hydroxyethyl) estra-[1,3,5]-triene

To a room temperature solution of 2-ethyl-3-O-benzyl-17-(1-ethenyl) estra-[1,3,5]-triene (700 mg, 1.4 mmol) as a mixture of geometric isomers) was added borane THF (16 mL, 1M). The reaction was stirred for 14 h at rt then treated with sodium hydroxide (20 mL, 10% aq) (causing vigorous gas evolution) and then hydrogen peroxide (60 mL, 27.5% aq). After 2 h further stirring the THF was removed on a rotary evaporator and the resultant mixture was extracted into ether (2×100 mL). The combined organic layers were then washed with water (2×100 mL) and brine (75 mL), dried and evaporated to give a colourless oil. The crude product was purified by column chromatography to give two fractions f1 (150 mg, R_{f} 0.3 in 15% ethyl acetate/hexane) and f2 (350 mg, R_{f} 0.22 in 15% ethyl acetate/hexane) (85% combined yield) which is assigned as a single diastereoisomer of the 17β-(1-hydroxyethyl) derivative of 2-ethyl-3-O-benzyl-17β-(1-hydroxyethyl) estra-[1,3,5]-triene (likely (S)-configuration at C-20) as a colourless oil which shows δ_{H} 0.65 (3H, s, 18-CH₃), 1.21 (3H, t, J 7.4, CH₂Me), 1.18-2.34 (21H, m, including 1.26 (3H, d, J 6.2, CH₃CH(OH)) and 1.20 (3H, t, J 7.4, CH₂Me)), 2.66 (2H, q, J 7.4, CH₂Me), 2.76-2.92 (2H, m, 6-CH₂), 3.69-3.79 (1H, m, CHOH), 5.04 (2H, s, OCH₂), 6.63 (1H, s, ArH), 7.09 (1H, s, ArH) and 7.29-7.44 (5H, m). C₂₉H₃₈O₂.

### 2-Ethyl-3-O-benzyl-17β-(acyl) estra-[1,3,5]-triene

To a stirred, 0° C., solution of 2-ethyl-3-O-benzyl-17β-(1-hydroxyethyl) estra-[1,3,5]-triene MPL06031f2 (330 mg, 0.77 mmol) in dichloromethane (20 mL) was added Dess Martin periodinane (392 mg, 1.2 eq, 0.92 mmol) in one portion. The reaction was stirred overnight and then diluted with ether (100 mL) and sodium hydroxide (2 mL, 1M aq) then stirred for a further 0.5 h prior to washing with water (100 mL) and brine (100 mL), drying and evaporating. The product, 2-Ethyl-3-O-benzyl-17β-(acyl) estra-[1,3,5]-triene, was obtained by adding hexane to the resultant oil as white needles (280 mg, 87%) m.p. 134-135° C. (R_{f} 0.45 in 4:1 hexane/ethyl acetate). δ_{H} 0.65 (3H, s, 18-CH₃), 1.21 (3H, t, J 7.4, CH₂Me), 1.25-2.40 (13H, m), 2.15 (3H, s, COCH₃), 2.66 (2H, q, J 7.4, CH₂Me), 2.59-2.71 (1H, m, 17α-H), 2.76-2.92 (2H, m, 6-CH₂), 5.04 (2H, s, OCH₂), 6.63 (1H, s, ArH), 7.10 (1H, s, ArH) and 7.28-7.45 (5H, m); δ_{C} 13.4, 14.6, 22.8, 23.5, 24.1, 26.7, 27.8, 29.7, 31.5, 38.8, 39.0, 43.7, 44.4, 55.6, 63.9, 69.8, 111.9, 126.1, 127.0, 127.6, 128.5, 130.3, 132.0, 134.9, 137.7, 154.5, 209.6. C₂₉H₃₆O₂.

### 2-Ethyl-3-hydroxy-17β-(acyl) estra-[1,3,5]-triene

A solution of 2-ethyl-3-O-benzyl-17β-(acyl) estra-[1,3,5]-triene (260 mg, 0.63 mmol) in THF (3 mL) and methanol (20 mL) was treated with Pd/C (10%, 50 mg) and stirred under H2 for 16 h. The reaction was then filtered through a pad of celite and evaporated to give the desired product, 2-ethyl 17β-acyl estrone, as a white solid (180 mg, 92%) which was then crystallised from ethyl acetate/hexane to give 2-ethyl-3-hydroxy-17β-(acyl) estra-[1,3,5]-triene as white needles m.p. 197-200° C. which showed δ_{H} 0.64 (3H, s, 18-CH₃), 1.21 (3H, t, J 7.4, CH₂Me), 1.24-1.90 (9H, m), 2.15 (3H, s, COCH₃), 2.12-2.40 (4H, m), 2.58 (2H, q, J 7.4, CH₂Me), 2.60 (1H, app t, J 9.4, 17α-H), 2.74-2.86 (2H, m, 6-CH₂), 4.72 (1H, s, OH), 6.49 (1H, s, ArH) and 7.03 (1H, s, ArH), δ_{C} 13.4, 14.4, 22.8, 23.0, 24.1, 36.7, 27.7, 29.2, 31.5, 38.8, 39.0, 43.7, 44.4, 55.6, 63.9, 115.2, 126.2, 127.2, 132.3, 135.4, 151.2 and 209.8. C₂₂H₃₀O₂.

### 2-Ethyl-3-O-sulfamoyl-17β-(acyl) estra-[1,3,5]-triene

A solution of 2-ethyl-3-hydroxy-17β-(acyl) estra-[1,3,5]-triene (80 mg, 0.26 mmol) in DMA (2 mL) was added to solid, ice bath cooled, sulfamoyl chloride (0.6 mmol). The reaction was stirred for 16 h then diluted with water and ethyl acetate (50 mL each). The organic layer was separated and washed with water (5×50 mL) and brine then dried and evaporated to give a white solid. The desired product 2-ethyl-3-O-sulfamoyl 17β-acyl estrone was purified by column chromatography (10% acetone in chloroform) to give 2-Ethyl-3-O-sulfamoyl-17β-(acyl) estra-[1,3,5]-triene as a white solid (95 mg, 91%). This material was crystallised from ethyl acetate/hexane to give fine white needles (73 mg first crop) m.p. 192-194° C. which showed δ_{H} 0.65 (3H, s, 18-CH₃), 1.21 (3H, t, J 7.4, CH₂Me), 1.24-1.93 (9H, m), 2.15 (3H, s, COCH₃), 2.15-2.40 (4H, m), 2.60 (1H, dd, J 9.4 and 9.0), 2.69 (2H, q, J 7.4, CH₂Me), 2.81-2.87 (2H, m, 6-CH₂), 4.93 (2H, s, NH₂), 7.07 (1H, s, ArH) and 7.17 (1H, s, ArH); δ_{C} 13.4, 14.6, 22.9, 23.1, 24.1, 26.5, 27.4, 29.1, 31.5, 38.3, 38.9, 43.9, 44.3, 55.6, 63.8, 121.4, 126.9, 133.6, 135.9, 139.2, 146.1 and 209.4. C₂₂H₃₁SO₄N.

### 2-Ethyl-3-O-sulfamate 17-O-mesyl estrone

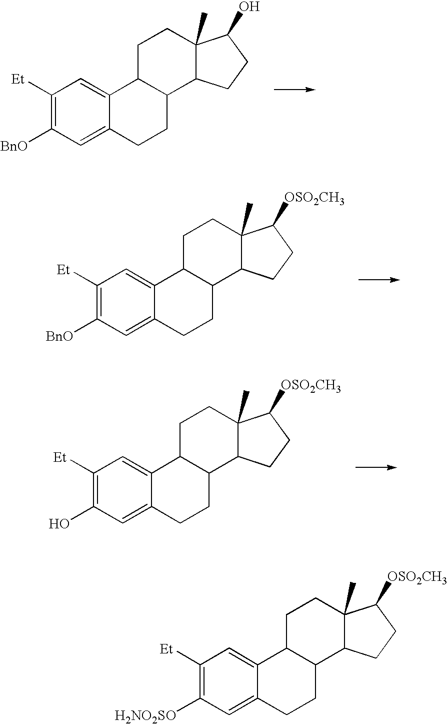

### 2-Ethyl-3-benzyloxy-17-O-mesyl estradiol

To a stirred 0° C. solution of 2-ethyl-3-O-benzyl estradiol (1 mmol) in dry pyridine (5 mL) was added methylsulfonyl chloride (0.09 mL, 1.2 mmol). The solution was stirred at 0° C. for 2 h before addition of water (20 mL). The organics were extracted into ethyl acetate (2×60 mL) and the combined organic layers were washed successively with water and brine then dried and evaporated. Column chromatography (hexane/ethyl acetate 5:1) afforded 2-ethyl-3-benzyloxy-17-O-mesyl estradiol as a white solid. 0.36 g (77%), mp=133° C. ¹H NMR (CDCl₃, 270 MHz): 0.87 (s, 3H, CH₃), 1.22 (t, J 7.4, 3H), 1.25-1.60 (m, 6H), 1.70-1.95 (m, 3H), 2.05 (m, 1H), 2.15-2.45 (m, 3H), 2.68 (q, J 7.4, 2H), 2.85 (m, 2H, H6), 3.02 (s, 3H, CH₃SO₂), 4.57 (m, 1H, H17), 5.05 (s, 2H, CH₂Ph), 6.64 (s, 1H, ArH, 7.10 (s, 1H, ArH), 7.36-7.44 (m, 5H, Ph). ¹³C NMR (CDCl₃): 11.7(CH₃), 14.6(CH₃), 23.0, 23.4, 26.0, 27.1, 27.9, 29.5 36.4, 38.2, 38.6, 43.3, 43.7, 49.0, 69.8 (CH₂Ph), 89.5(C17), 111.8, 126.2, 127.0, 127.6, 128.4 130.3, 131.7, 134.7, 137.6 and 154.5

### 2-Ethyl-17-O-mesyl estradiol

To a solution of 2-ethyl-3-benzyloxy-17-O-mesyl estradiol (0.5 mmol) in THF (10 mL) and ethanol (40 mL) was added 10% Pd/C (30 mg) and the mixture was then stirred at room temperature under hydrogen for 14 hours. The suspension was then filtered through celite and evaporated. After column chromatography (hexane/ethyl acetate 1:0 to 2:1) 2-ethyl-17-O-mesyl estradiol was isolated as a white solid. 145 mg (77%), mp=195° C. ¹H NMR (CDCl₃, 270 MHz): 0.86 (s, 3H, CH₃), 1.21 (t, J_{H-H}=7.7 Hz, 3H, CH₃), 1.25-1.60 (m, 6H), 1.71-1.91 (m, 3H), 2.03 (m, 1H), 2.13-2.38 (m, 3H), 2.58 (q, J_{H-H}=7.7 Hz, 2H, CH₂), 2.79 (m, 2H, H6), 3.01 (s, 3H, CH₃SO₂), 4.53 (s, 1H, OH), 4.56 (dd, 1 J_{H-H}=9.1 and 7.9 Hz, 1H, H17), 6.49 (s, 1H, ArH, 7.03 (s, 1H, ArH). ¹³C NMR (CDCl₃): 11.7(CH₃), 14.6(CH₃), 23.0, 23.4, 26.0, 27.1, 27.9, 29.5 36.4, 38.2, 38.6, 43.3, 43.7, 49.0, 89.5(C17), 115.2, 126.3, 127.3, 132.1, 135.2 and 151.2 MS m/z: 350.16 (M⁺) HPLC 100%. Microanalysis: C, 66.30 (expected 66.63); H, 7.80 (expected 7.99)

### 2-Ethyl-3-O-sulfamoyl-17-O-mesyl estradiol

Sulfamoyl chloride (0.6 mmol) was dissolved in DMA (1 mL), cooled to 0° C., and then treated with 2-ethyl-17-O-mesyl estradiol (0.2 mmol) under nitrogen. The solution was stirred for 15 hours at room temperature before addition of water (5 mL) and extraction into ethyl acetate (2×50 mL). The organic layer washed successively with water and brine, dried and evaporated to give the crude product. After column chromatography (hexane/ethyl acetate 5:2) 2-ethyl-3-O-sulfamoyl-17-O-mesyl estradiol was obtained as a white solid. 60 mg (66%) mp=179° C. ¹H NMR (CDCl₃, 270 MHz): 0.85 (s, 3H, CH₃), 1.20 (t, J_{H-H}=7.4 Hz, 3H, CH₃), 1.30-1.55 (m, 6H), 1.73-187 (m, 3H), 2.04 (m, 1H), 2.16-2.36 (m, 3H), 2.68 (q, J_{H-H}=7.4 Hz, 2H, CH₂), 2.82 (m, 2H, H6), 3.01 (s, 3H, CH₃SO₂), 4.57 (dd, 1 J_{H-H}=8.7 and 8.1 Hz, 1H, H17), 5.08 (s, 2H, NH₂), 6.49 (s, 1H, ArH, 7.03 (s, 1H, ArH). ¹³C NMR (CDCl₃): 14.1(CH₃), 17.0(CH₃), 25.4, 23.4, 28.2, 29.2, 30.3, 31.4 38.6, 40.5, 40.6, 45.6, 46.3, 51.4, 91.5(C17), 123.6, 129.2, 135.9, 137.9, 141.1 and 148.3. LRMS m/z: 457.32 (M⁺); HPLC 100%; Microanalysis: C, 53.40 (expected 55.12); H, 6.38 (expected 6.34); N, 3.09 (expected 3.06).

### 2-Substituted-3-O-sulfamoyl-17-N-sulfamoyl estradiol derivatives

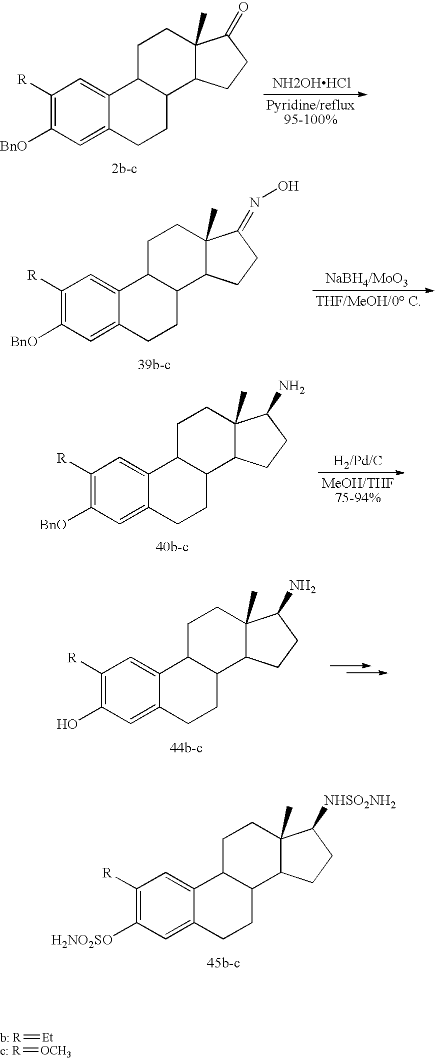

### 2-Substituted 17β-amino-estradiol 44b and 44c

A solution of 40b-c (1 mmol) in THF (5 mL) and methanol (20 mL) was stirred with 5% Pd/C (50 mg) under hydrogen for 24 hours. The suspension was filtered through celite and the solvents evaporated under reduced pressure. The crude oil was then purified by flash chromatography (ethyl acetate/methanol/TEA 20/1/0.2).

17β-amino-2-ethylestradiol 44b White solid, Mp=203-204° C., 285 mg (95%). R_{f}: 0.18 (ethyl acetate/methanol/Et3N, 10:1:0.2), ¹H NMR (270 MHz, DMSO-d₆) δ 0.73 (3H, s, CH₃), 1.07 (3H, t, J=7.4 Hz, CH₂CH₃), 1.13-1.36 (6H, m), 1.56-1.76 (3H, m), 1.98-2.08 (3H, m), 2.27 (1H, m), 2.45 (2H, q, J=7.4 Hz, CH₂CH₃), 2.66 (2H, m, H6), 2.96 (1H, t, J=8.9 Hz, H17), 6.44 (1H, s, ArH), 6.91 (1H, s, ArH), 7.76 (1H, br, OH), 8.88 (2H, br, NH₂). LC/MS (APCI−) tᵣ=2.37 min m/z 298.36 (M⁺−H) (MeOH/Water 95/5)

17β-amino-2-methoxyestradiol 44c White powder, mp=220-221° C., 270 mg (90%). mp 220-221° C. R_{f}: 0.16 (ethyl acetate/methanol/TEA 10:1:0.2). ¹H NMR (270 MHz, DMSO-d6) δ 0.59 (3H, s, CH₃), 1.05-1.39 (8H, m), 1.55-1.63 (1H, m), 1.72-1.95 (3H, m), 2.02-2.13 (1H, m), 2.22-2.30 (1H, m), 2.62 (2H, m, H6), 3.70 (3H, s, CH₃O), 6.43 (1H, s, ArH), 6.76 (1H, s, ArH). ¹³C NMR (100 MHz, DMSO-d6) δ 11.6 (CH₃), 23.5, 26.3, 26.7, 27.8, 28.9, 31.6, 37.0, 43.1, 44.4, 52.0, 56.2 (CH₃O), 63.2, 110.1, 116.0, 128.8, 130.8, 144.8 and 146.0; LC/MS (APCI−) tᵣ=1.96 min m/z 300.38 (M⁺−H) (MeOH/Water 95/5).

### 2-Substituted 17β-(N-sulfamoyl)-estradiol

A solution of the appropriate 2-substituted 17β-amino estradiol (44b or 44c) (0.5 mmol) and sulfamide (149 mg, 2.5 mmol) in 1,4-dioxane (5 mL) was refluxed for 5 h and the solvent was evaporated under reduced pressure. After addition of ethyl acetate and water, the organic layer was separated washed with water, brine, dried (MgSO₄) and concentrated under reduced pressure. The crude solid was purified by flash chromatography (hexane/ethyl acetate 4:1 to 2:1) to give a white powder which was recrystallized from ethyl acetate/hexane 1:1.

2-Ethyl 17β-(N-sulfamoyl)-estradiol. White solid, mp 236-237° C., 115 mg 60%); R_{f}: 0.32 (ethyl acetate/hexane 1:1). ¹H NMR (300 MHz, CDCl₃/DMSO-d₆ 20:1) δ 0.66 (3H, s, CH₃), 1.12 (3H, t, J=7.3 Hz, CH₂CH₃), 1.15-1.52 (7H, m), 1.61-1.82 (2H, m), 1.90-1.96 (1H, m), 2.06-2.28 (3H, m), 2.52 (2H, q, J=7.3 Hz, CH₂CH₃), 2.68 (2H, m, 116), 3.26 (1H, q, J=9.0 Hz, H17), 4.90 (1H, d, J=9 Hz, NH), 5.30 (2H, s, NH₂), 6.47 (1H, s, ArH), 6.93 (1H, s, ArH), 7.73 (1H, br, OH). LC/MS (APCI−) tᵣ=4.12 min m/z 377.39 (M⁺+H). (gradient MeOH/H₂O from 50/50 to 95/5 in 5 min).

2-Methoxy 17β-(N-sulfamoyl)-estradiol. White solid, mp 196-197° C., 117 mg (60%) R_{f}: 0.20 (ethyl acetate/hexane 1:1). ¹H NMR (300 MHz, CDCl₃/DMSO-d₆ 20:1) δ 0.65 (3H, s, CH₃), 1.10-1.52 (7H, m), 1.61-1.82 (2H, m), 1.89-1.94 (1H, m), 2.08-2.25 (3H, m), 2.63-2.71 (2H, m, H6), 3.25 (1H, q, J=9.0 Hz, H17), 4.89 (1H, d, J=9 Hz, NH), 5.27 (2H, s, NH₂), 6.50 (1H, s, ArH), 6.52 (1H, s, ArH), 6.69 (1H, br, OH). ¹³C NMR (100 MHz, CDCl₃/DMSO-d₆ 10:1) δ 12.0 (CH₃), 23.2, 26.5, 27.4, 28.9, 29.3, 36.7, 38.9, 42.8, 44.2, 51.3, 56.2, 63.7, 112.2, 125.7, 131.5, 138.8, 141.7 and 151.2. LC/MS (APCI−) tᵣ=3.85 min m/z 379.41 (M⁺+H). (gradient MeOH/H₂O from 50/50 to 95/5 in 5 min); HRMS(FAB+): found 380.177246 for calcd. C₁₉H₂₈N₂O₄S 380.176979

### 2-Methoxyestradiol-3,17-O,N-bis-sulfamate

A solution of 17β-(N-sulfamoyl)-2-methoxy estradiol (90 mg, 0.24 mmol) and sulfamoyl chloride (0.48 mmol) in DMA (1 mL) was stirred for 24 h at rt. After removal of DMA under vacuum, the crude mixture was subjected to flash chromatography (Hexane/EtOAc 1:1 to 1:2) to give the desired bis-sulfamate 25 mg (23%) as a white powder mp 128-129° C. R_{f}: 0.14 (Hexane/EtOAc 1:1). ¹H NMR (270 MHz, CD₃COCD₃) δ 0.79 (3H, s, CH₃), 1.21.-1.54 (7H, m), 1.56-1.80 (2H, m), 1.85-1.93 (1H, m), 2.20-2.42 (3H, m), 2.77 (2H, m, H6), 3.32 (1H, m, H17), 3.83 (3H, s, CH₃O), 5.53 (1H, d, J=8.9 Hz, NH), 5.82 (2H, s, NH₂), 6.91 (2H, br, NH2), 6.99 (1H, s, ArH), 7.02 (1H, s, ArH). ¹³C NMR (67.5 MHz, CD₃COCD₃) δ 11.5 (CH₃), 23.1, 26.2, 27.2, 36.8, 38.8, 42.7, 44.7, 46.0, 51.5, 55.5, 63.6, 63.7, 110.4, 123.8, 128.8, 137.2, 139.6 and 150.0; LC/MS (APCI−) tᵣ=1.29 min m/z 458.04 (M⁺−H) (MeOH/Water 50/50); HPLC tᵣ=1.79 min (100%). (MeOH/H₂O 70/30)

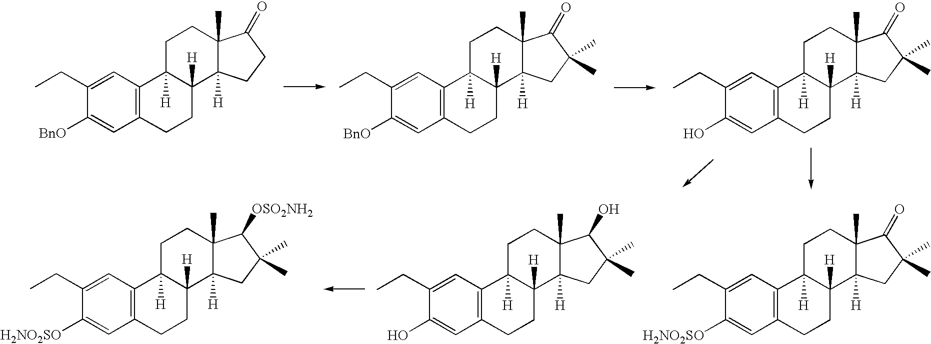

### 2-Ethyl-3-O-benzyl-16-dimethyl estrone

A solution of 2-ethyl-3-O-benzyl estrone (776 mg, 2 mmol) in THF (30 mL) was treated with sodium hydride (240 mg, 6 mmol) and then methyl iodide (1.25 mL, 20 mmol). The reaction was brought to reflux for 14 h and then treated with further aliquots of sodium hydride (240 mg) and methyl iodide (1.25 mL). After refluxing for a further 24 h the reaction was cooled to rt, quenched with ammonium chloride and diluted in ethyl acetate (70 mL). The organic layers were then separated, washed with water (2×50 mL), brine (50 mL), dried and evaporated. A pure fraction of the desired product, 2-ethyl-3-O-benzyl-16-dimethyl estrone, was purified by column chromatography (5% ethyl acetate in hexane) to give a white foam (400 mg, 48%) which showed δ_{H} 0.93 (3H, s, 18-CH₃), 1.08 (3H, s, 16-CH₃), 1.20 (3H, s, 16-CH₃), 1.21 (3H, t, J 7.4, CH₂Me) 1.34-2.48 (11H, m), 2.66 (2H, q, J 7.4, CH₂Me), 2.83-2.92 (2H, m, 6-CH₂), 5.05 (2H, s, OCH₂), 6.65 (1H, s, ArH), 7.11 (1H, s, ArH) and 7.28-7.47 (5H, m); δ_{C} 14.5, 14.6, 23.4, 25.9, 26.0, 26.8, 27.3, 29.6, 32.4, 37.6, 37.9, 44.2, 45.3, 47.2, 49.1, 69.8, 111.8, 126.1, 127.0, 127.6, 128.5, 130.4, 131.8, 134.7, 137.6, 154.6 and 225.3. C₂₉H₃₆O₂

### 2-Ethyl-16-dimethyl estrone

A degassed solution of 2-ethyl-3-O-benzyl-16-dimethyl estrone (360 mg, 0.86 mmol) in THF (3 mL) and methanol (25 mL) was treated with 10% Pd/C (50 mg) and placed under an hydrogen atmosphere for 16 h. The reaction mixture was then filtered through celite and evaporated to give 2-ethyl-16-dimethyl estrone as a white solid (270 mg, 96%) m.p. 196-198° C. which showed δ_{H} 0.92 (3H, s, 18-CH₃), 1.06 (3H, s, 16-CH₃), 1.20 (3H, s, 16-CH₃), 1.21 (3H, t, J 7.4, CH₂Me) 1.30-2.44 (11H, m), 2.58 (2H, q, J 7.4, CH₂Me), 2.78-2.86 (2H, m, 6-CH₂), 4.63 (1H, s, OH), 6.50 (1H, s, ArH), and 7.04 (1H, s, ArH); δ_{C} 14.3, 14.5, 23.0, 25.9, 26.0, 26.7, 27.3, 29.1, 32.3, 37.6, 37.9, 44.2, 45.3, 47.2, 126.3, 127.3, 132.1, 135.2, 135.7, 144.7, 151.2 and 203.1. C₂₂H₃₀O₂

### 2-Ethyl-3-O-sulfamoyl-16-dimethyl estrone

To an ice bath cooled solution of sulfamoyl chloride (0.6 mmol) in DMA (1.5 mL) was added 2-ethyl-16-dimethyl estrone (84 mg, 0.26 mmol). After 3 h the reaction was diluted with ethyl acetate (20 mL) and water (20 mL). The organic layer was separated and washed with water (5×20 mL) and brine (20 mL) then dried and evaporated to give a colourless oil. The desired product, 2-ethyl-3-O-sulfamoyl-16-dimethyl estrone, was purified by column chromatography (eluant 9% acetone in chloroform) as a colourless oil (64 mg, 61%) and then precipitated from ethyl acetate/hexane as a white powder m.p. 93-95° C. which showed δ_{H} 0.92 (3H, s, 18-CH₃), 1.07 (3H, s, 16-CH₃), 1.20 (3H, s, 16-CH₃), 1.21 (3H, t, J 7.4, CH₂Me), 1.34-2.45 (11H, m), 2.69 (2H, q, J 7.4, CH₂Me), 2.83-2.93 (2H, m, 6-CH₂), 4.97 (2H, s, NH₂), 7.09 (1H, s, ArH) and 7.18 (1H, s, NH₂). C₂₂H₃₁NSO₄

### 2-Ethyl-16-dimethyl estradiol

A solution of 2-ethyl-16-dimethyl estrone (185 mg, 0.56 mmol) in THF (15 mL) was treated with lithium aluminium hydride (95 mg, 2.5 mmol) at room temperature. After 1 h the reaction was quenched by adding sodium hydroxide (5 mL) and stirring for 0.5 h. After standard work-up the desired product 2-ethyl-16-dimethyl estradiol was obtained as a white powder (180 mg, 98%) m.p. 176-178° C. which showed δ_{H} 0.78 (3H, s, 18-CH₃), 1.01 (3H, s, 16-CH₃), 1.08 (3H, s, 16-CH₃), 1.14-1.60 (1H, m including 1.18 (3H, t, J 7.4, CH₂Me), 1.76-1.96 (2H, m), 2.14-2.36 (2H, m), 2.55 (2H, q, J 7.4, CH₂Me), 2.70-2.82 (2H, m, 6-CH₂), 3.27 (1H, s, 17αH), 4.50-4.60 (1H, br, OH), 6.48 (1H, s, ArH), and 7.05 (1H, s, ArH). C₂₂H₃₂O₂

### 2-Ethyl-16-dimethyl-3,17-O,O-bis-sulfamoyl estradiol

To an ice bath cooled solution of sulfamoyl chloride (1.2 mmol) in DMA (3 mL) was added 2-ethyl-16-dimethyl estradiol (95 mg, 0.29 mmol). After 3 h the reaction was diluted with ethyl acetate (20 mL) and water (20 mL). The organic layer was separated and washed with water (5×20 mL) and brine (20 mL) then dried and evaporated to give a white solid. The desired product 2-ethyl-16-dimethyl-3,17-O,O-bis-sulfamoyl estradiol was purified by chromatography to give a white powder (102 mg, 72%) which was then crystallised from ethyl acetate/hexane to give white needles m.p. 193-195° C. which showed δ_{H} (d₆-DMSO) 0.80 (3H, s, 18-CH₃), 1.03 (3H, s, 16-CH₃), 1.12 (3H, t, J 7.4, CH₂Me), 1.14 (3H, s, 16-CH₃), 1.21-1.58 (7H, m), 1.72-2.14 (2H, m), 2.14-2.37 (2H, m), 2.62 (2H, q, J 7.4, CH₂Me), 2.74-2.84 (2H, m, 6-CH₂), 4.00 (1H, s, 17-αH), 6.99 (1H, s,), 7.21 (1H, s), 7.47 (2H, s, NH₂), 7.96 (2H, s, NH₂). C₂₂H₃₄N₂O₆S₂

Biological Data

The following biological data were obtained using the Protocols described herein.

Inhibition of MCF-7 Cell Proliferation

| Compound No. | Structure | % Inhibition of MCF-7 proliferation at 10 μM, |
|---|---|---|
| 8 | 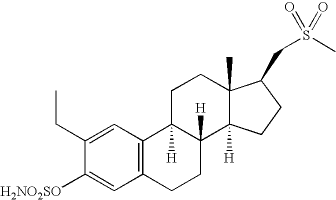 | 79 |
| 11 | 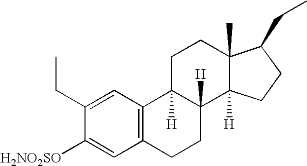 | 83 |
| 14 | 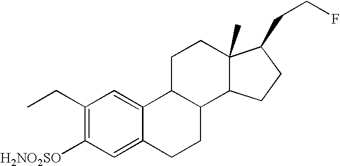 | 89 |
| 23 | 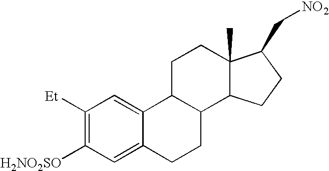 | <50 |
| 29 | 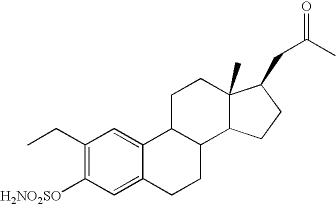 | 69 |

| Compound No. | Structure | Concentration for 50% inhibition of MCF-7 proliferation (μM) |
|---|---|---|
| 57 | 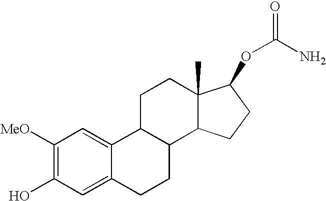 | 44 |
| 58 | 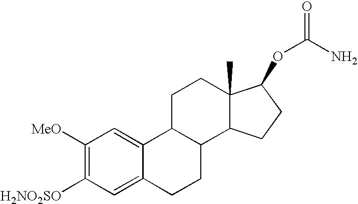 | 1.8 |

Effects on Tubule Formation

The effects of drugs on tubule formation (measured as a marker of their anti-angiogenic potential) was assessed using an Angiogenesis kit (TCS-Cellworks Ltd (Bucks, UK). For this, human umbilical vein endothelial cells (HUVECs) were cultured in a 24-well plate within a matrix of human diploid fibroblasts of dermal origin. The co-cultured cells were incubated throughout the experiment at 37° C. under 5% CO₂ in a humidified incubator. On day 1, the culture medium was removed and replaced with medium containing the drugs under investigation. On days 4, 7 and 9, the medium was replaced with fresh medium containing the drugs under investigation. On day 11, the cells were washed with PBS and 70% ethanol (1 ml) added to each well for 30 min to fix the cells. After fixation, the cells were washed with blocking buffer (1 ml PBS+1% bovine serum albumin, Sigma, UK) and stained with either von Willebrand's factor or CD31. The extent of tubule formation was quantified by manual scoring or by computer analysis. Images were captured using a Kodak DC120 digital camera. In addition, details of changes in tubule formation induced by drugs were also recorded by high definition scanning of plates with some of the scans being presented as Photoshop processed images.

Most solid tumours can only grow beyond 1-2 mm in size if they develop a blood vessel network so that they can obtain essential nutrients to support their growth (a process known as angiogenesis). Drugs that block this angiogenic process should therefore inhibit the growth of a wide range of solid tumours.

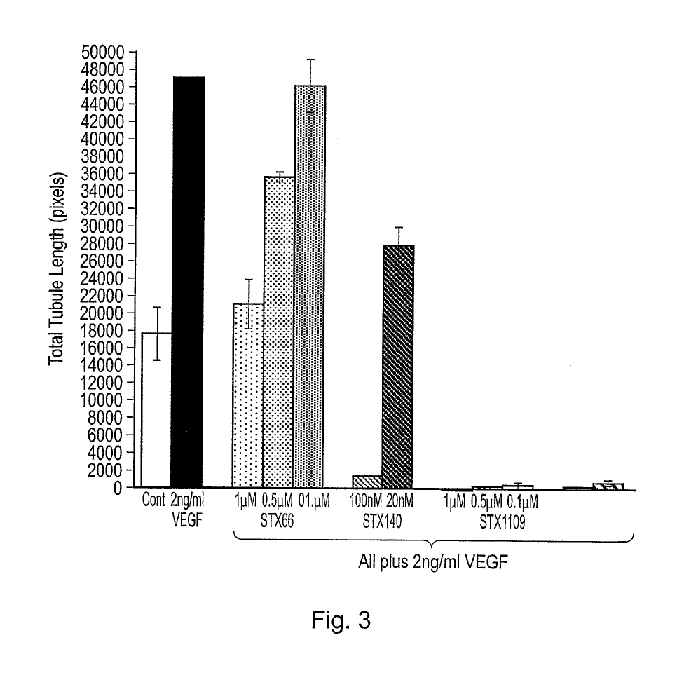

In this assay, the ability of STX1109 (and related compounds) to act as an inhibitor of angiogenesis was examined using a co-culture of HUVECs and dermal fibroblasts. In this system, the endothelial cells initially form small islands within the fibroblast matrix. They subsequently proliferate and enter a migratory phase during which they move through the matrix to form thread-like tubule structures. These coalesce to form a network of anastomosing tubules. The extent of inhibition of tubule formation can be quantified by computer analysis (FIG. 3). As shown STX1109, at 1 μM, 0.5 μM and 0.1 μM completely inhibited tubule formation confirming the anti-angiogenic potential of this compound.

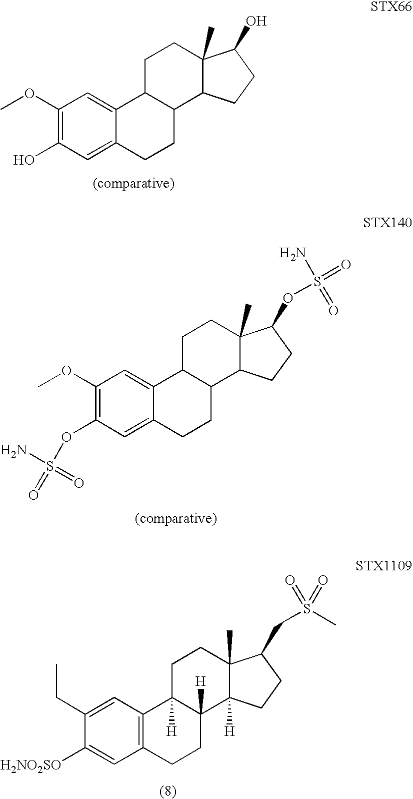

All publications and patents mentioned in the above specification are herein incorporated by reference.

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

The invention will now be further described by the following numbered paragraphs:

1. A compound comprising a steroidal ring system and an optional group R¹ selected from any one of —OH, a sulphamate group, a phosphonate group, a thiophosphonate group, a sulphonate group or a sulphonamide group;

wherein the A ring of the steroidal ring system is optionally substituted at position 2 or 4 with a group R⁴ which may be a suitable subtituent

wherein the D ring of the steroidal ring system is substituted by a group R² of the formula -L-R³, wherein L is an optional linker group and R³ is selected from groups which are or which comprise one of

(i) —SO₂R⁵, wherein R⁵ is H, a hydrocarbyl group or a bond or group attached to the D ring

(ii) —NO₂

(iii) —SOR⁶, wherein R⁶ is H or a hydrocarbyl group

(iv) —R⁷, wherein R⁷ is a halogen

(v) -alkyl

(vi) —C(═O)R³, wherein R³ is H or hydrocarbyl

(vii) —C≡CR⁹, wherein R⁹ is H or hydrocarbyl

(viii) —OC(═O)NR¹⁰R¹¹ wherein R¹⁰ and R¹¹ are independently selected from H and hydrocarbyl 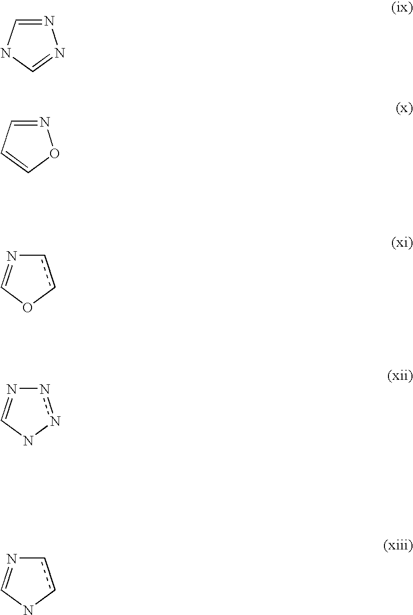
wherein when R³ is -alkyl, R⁴ is present as a hydrocarbon group, when R³ is —NO₂R⁴ is present and/or R¹ is present as a sulphamate group, and when R³ is —C(═O)R³R⁴ is present and R¹ is present as a sulphamate group.
2. A compound according to paragraph 1 of Formula I 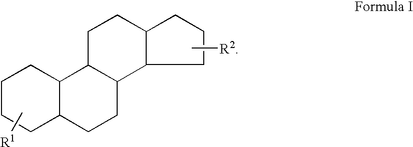
3. A compound according to paragraph 1 of Formula II 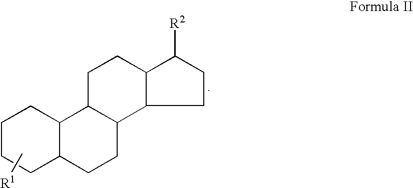
4. A compound according to paragraph 1 of Formula III 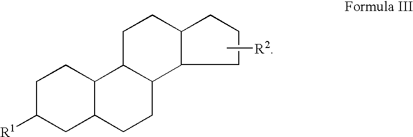
5. A compound according to paragraph 1 of Formula IVa or Formula IVb 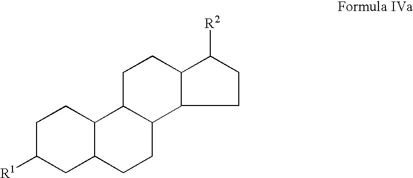 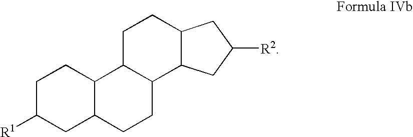
6. A compound according to paragraph 1 of Formula IVc 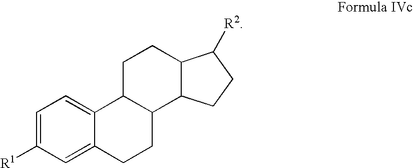
7. A compound according to any one of the preceding paragraphs wherein R⁴ is a hydrocarbyl group or an oxyhydrocarbyl group.
8. A compound according to paragraph 7 wherein R⁴ is an alkoxy group.
9. A compound according to paragraph 8 wherein R⁴ is methoxy.
10. A compound according to any one of paragraphs 1 to 6 wherein R⁴ is an hydrocarbon group.
11. A compound according to paragraph 10 wherein R⁴ is an alkyl group.
12. A compound according to paragraph 11 wherein R⁴ is ethyl.
13. A compound according to any one of the preceding paragraphs wherein R⁴ is at position 2 of the A ring.
14. A compound according to any one of the preceding paragraphs wherein when the A ring is substituted with R¹ and R⁴, R⁴ is ortho substituted with respect to R¹.
15. A compound according to any one of the preceding paragraphs wherein R¹ is present.
16. A compound according to any one of the preceding paragraphs wherein R¹ is —OH or a sulphamate group.
17. A compound according to any one of the preceding paragraphs wherein R¹ is —OH.
18. A compound according to any one of paragraphs 1 to 16 wherein R¹ is a sulphamate group.
19. A compound according to paragraph 18 wherein R¹ is a sulphamate group of the formula 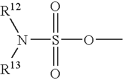
wherein R¹² and R¹³ are independently selected from H, alkyl, cycloalkyl, alkenyl and aryl, or combinations thereof, or together represent alkylene, wherein the or each alkyl or cycloalkyl or alkenyl or aryl optionally contains one or more hetero atoms or groups.
20. A compound according to paragraph 19 wherein at least one of R¹² and R¹³ is H.
21. A compound according to paragraph 20 wherein each of R¹² and R¹³ is H.
22. A compound according to any one of the preceding paragraphs wherein L is selected from a hydrocarbyl group, —NR¹⁴— and —O—, wherein R¹⁴ is H, a hydrocarbyl group or a bond.
23. A compound according to paragraph 22 wherein L is selected from a hydrocarbon group, —NR¹⁴— and —O—.
24. A compound according to paragraph 22 wherein L is selected from an alkylene group, —NR¹⁴— and —O—.
25. A compound according to paragraph 22 wherein L is selected from a C₁₋₁₀ alkylene group, —NR¹⁴— and —O—.
26. A compound according to paragraph 22 wherein L is selected from a C₁ or C₂ alkylene group, —NR¹⁴— and —O—.
27. A compound according to any one of the preceding paragraphs wherein groups (ix) to (xiii) are selected from optionally substituted groups of the formulae 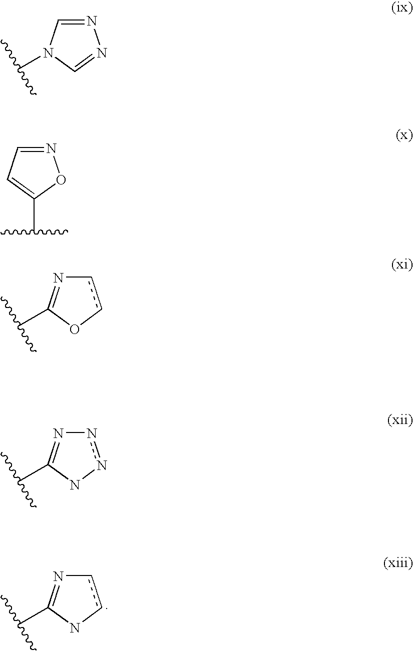
28. A compound according to any one of the preceding paragraphs wherein R³ is —SO₂R⁵, wherein R⁵ is H, a hydrocarbyl group or a bond or group attached to the D ring.
29. A compound according to paragraph 28 wherein R⁵ is selected from H and C₁₋₁₀ alkyl.
30. A compound according to paragraph 28 wherein R⁵ is selected from H and C₁₋₅ alkyl.
31. A compound according to paragraph 28 wherein R⁵ is selected from H and C₁₋₃ alkyl.
32. A compound according to paragraph 28 wherein R⁵ is —CH₃.
33. A compound according to paragraph 28 wherein R⁵ is —O—R¹⁵-D, wherein R¹⁵ is a linker and D is a member of the D ring.
34. A compound according to paragraph 33 wherein R⁵ is —O—R¹⁵-D, wherein R¹⁵ is selected from —O—CH₂— and —N═CH—, and wherein D is a member of the D ring.
35. A compound according to paragraph 27 wherein R² is —CH₂—R³ or —NH—R³.
36. A compound according to any one of paragraphs 1 to 26 wherein R³ is —NO₂.
37. A compound according to paragraph 36 wherein R² is —CH₂—R³.
38. A compound according to any one of paragraphs 1 to 26 wherein R³ is —SOR⁶, wherein R⁶ is H or a hydrocarbyl group.
39. A compound according to paragraph 38 wherein R⁶ is selected from H and C₁₋₁₀ alkyl.
40. A compound according to paragraph 39 wherein R⁶ is —CH₃.
41. A compound according to paragraph 38 wherein R² is —CH₂—R³.
42. A compound according to any one of paragraphs 1 to 26 wherein R³ is —R⁷, wherein R⁷ is a halogen.
43. A compound according to paragraph 42 wherein R⁷ is fluorine.
44. A compound according to paragraph 42 wherein R² is —CH₂CH₂—R³.
45. A compound according to any one of paragraphs 1 to 26 wherein R³ is -alkyl
46. A compound according to paragraph 45 wherein R³ is C₁₋₁₀ alkyl.
47. A compound according to paragraph 45 wherein R³ is C₁₋₅ alkyl.
48. A compound according to paragraph 45 wherein R³ is —CH₃ or —CH₂CH₃.
49. A compound according to paragraph 45 wherein R² is R³.
50. A compound according to any one of paragraphs 1 to 26 wherein R³ is —C(═O)R³, wherein R³ is H or hydrocarbyl.
51. A compound according to paragraph 50 wherein R³ is selected from H and C₁₋₁₀ alkyl.
52. A compound according to paragraph 50 wherein R⁸ is —CH₃.
53. A compound according to paragraph 50 wherein R² is —CH₂—R³.
54. A compound according to any one of paragraphs 1 to 26 wherein R³ is —C≡CR⁹, wherein R⁹ is H or hydrocarbyl.
55. A compound according to paragraph 54 wherein R⁹ is selected from H and C₁₋₁₀ alkyl.
56. A compound according to paragraph 54 wherein R⁹ is —CH₃.
57. A compound according to paragraph 54 wherein R² is —CH₂—R³.
58. A compound according to any one of paragraphs 1 to 26 wherein R³ is —OC(═O)NR¹⁰R¹¹, wherein R¹⁰ and R¹¹ are independently selected from H and hydrocarbyl.
59. A compound according to paragraph 58 wherein R¹⁰ and R¹¹ are independently selected from H and C₁₋₁₀ alkyl.
60. A compound according to paragraph 58 wherein R¹⁰ and R¹¹ are both H.
61. A compound according to paragraph 58 wherein R² is R³.
62. A compound according to any one of paragraphs 1 to 26 wherein R³ is 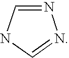
63. A compound according to 62 wherein R³ is 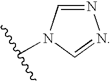
64. A compound according to paragraph 62 wherein R² is selected from —CH₂CH₂—R³, ═N—R³ and —NH—R³.
65. A compound according to any one of paragraphs 1 to 26 paragraphs wherein R³ is 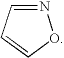
66. A compound according to 65 wherein R³ is 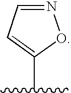
67. A compound according to 65 wherein R³ is 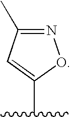
68. A compound according to paragraph 65 wherein R² is selected from ═CH—R³ and —CH₂CH₂—R³. 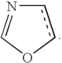
69. A compound according to any one of paragraphs 1 to 26 wherein R³ is
70. A compound according to 68 wherein R³ is 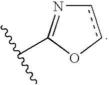
71. A compound according to paragraph 68 wherein R² is selected from ═CH—R³ and —CH₂CH₂—R³.
72. A compound according to any one of paragraphs 1 to 26 wherein R³ is 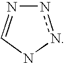
73. A compound according to 72 wherein R³ is 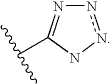
74. A compound according to 73 wherein R³ is selected from 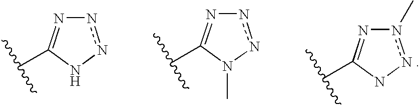
75. A compound according to paragraph 72 wherein R² is selected from ═CH—R³ and —CH₂CH₂—R³.
76. A compound according to any one of paragraphs 1 to 26 wherein R³ is 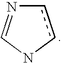
77. A compound according to 76 wherein R³ is
78. A compound according to paragraph 76 wherein R² is selected from ═CH—R³ and —CH₂CH₂—R³.
79. A compound according to any one of the preceding paragraphs wherein group R² is in an α configuration.
80. A compound according to any one of the preceding paragraphs wherein group R² is in an α configuration on the 17 position of the D ring.
81. A compound according to any one of paragraphs 1 to 79 wherein group R² is in an α configuration on the 16 position of the D ring.
82. A compound according to any one of the preceding paragraphs wherein R¹ is a sulphamate group and the compound is suitable for use as an inhibitor of oestrone sulphatase (E.C. 3.1.6.2).
83. A compound according to paragraph 82 wherein if the sulphamate group on the sulphamate compound were to be replaced with a sulphate group to form a sulphate compound then the sulphate compound would be hydrolysable by a steroid sulphatase enzyme (E.C.3.1.6.2).
84. A compound according to paragraph 83 wherein if the sulphamate group on the sulphamate compound were to be replaced with a sulphate group to form a sulphate compound and incubated with a steroid sulphatase enzyme (E.C.3.1.6.2) at a pH 7.4 and 37° C. it would provide a K, value of less than 50 μM.
85. A compound according to paragraph 83 wherein if the sulphamate group on the sulphamate compound were to be replaced with a sulphate group to form a sulphate compound and incubated with a steroid sulphatase enzyme (E.C.3.1.6.2) at a pH 7.4 and 37° C. it would provide a K, value of less than 50 μM.
86. A pharmaceutical composition comprising:
(a) a compound as defined in any one of paragraphs 1 to 85, and
(b) a pharmaceutically acceptable carrier, diluent, excipient or adjuvant.
87. A compound as defined in any one of paragraphs 1 to 85 for use in medicine.
88. Use of a compound as defined in any one of paragraphs 1 to 85 in the manufacture of a medicament to prevent and/or inhibit tumour growth.
89. Use of a compound as defined in any one of paragraphs 1 to 85 in the manufacture of a medicament for use in the therapy of a condition or disease associated with one or more of steroid sulphatase (STS) activity; cell cycling; apoptosis; cell growth; glucose uptake by a tumour; tumour angiogenesis; microtubules formation; and apoptosis.
90. Use of a compound as defined in any one of paragraphs 1 to 85 in the manufacture of a medicament for use in the therapy of a condition or disease associated with one or more of adverse steroid sulphatase (STS) activity; cell cycling; apoptosis; cell growth; glucose uptake by a tumour; tumour angiogenesis; microtubules formation; and apoptosis.
91. Use of a compound as defined in any one of paragraphs 1 to 85 in the manufacture of a medicament for one or more of inhibiting steroid sulphatase (STS) activity; modulating cell cycling; modulating apoptosis;-;modulating cell growth; preventing and/or suppressing glucose uptake by a tumour; preventing and/or inhibiting tumour angiogenesis; disrupting microtubules; and inducing apoptosis.
92. Use of a compound as defined in any one of paragraphs 1 to 85 in the manufacture of lo a medicament for inhibiting steroid sulphatase (STS) activity.
93. Use of a compound as defined in any one of paragraphs 1 to 85 in the manufacture of a medicament for modulating cell growth.
94. A method of treatment comprising administering to a subject in need of treatment a compound as defined in any one of paragraphs 1 to 85.
95. A method of treatment comprising administering to a subject in need of treatment a compound as defined in any one of paragraphs 1 to 85 in order to inhibit steroid sulphatase (STS) activity; modulate cell cycling; modulate apoptosis; modulator cell growth; prevent and/or suppress glucose uptake by a tumour; prevent and/or inhibit tumour angiogenesis; disrupt microtubules; and/or induce apoptosis.
96. A compound as substantially hereinbefore described with reference to the Examples.
97. A composition as substantially hereinbefore described with reference to the Examples.
98. A use as substantially hereinbefore described with reference to the Examples.
99. A method as substantially hereinbefore described with reference to the Examples.

1. A compound comprising a steroidal ring system and an optional group R¹ selected from any one of —OH, a sulphamate group, a phosphonate group, a thiophosphonate group, a sulphonate group or a sulphonamide group;
wherein the A ring of the steroidal ring system is optionally substituted at position 2 or 4 with a group R⁴ which may be a suitable subtituent
wherein the D ring of the steroidal ring system is substituted by a group R² of the formula -L-R³, wherein L is an optional linker group and R³ is selected from groups which are or which comprise one of
(i) —SO₂R⁵, wherein R⁵ is H, a hydrocarbyl group or a bond or group attached to the D ring
(ii) —NO₂
(iii) —SOR⁶, wherein R⁶ is H or a hydrocarbyl group
(iv) —R⁷, wherein R⁷ is a halogen
(v) -alkyl
(vi) —C(═O)R⁸, wherein R⁸ is H or hydrocarbyl
(vii) —C≡CR⁹, wherein R⁹ is H or hydrocarbyl
(viii) —OC(═O)NR¹⁰R¹¹ wherein R¹⁰ and R¹¹ are independently selected from H and hydrocarbyl 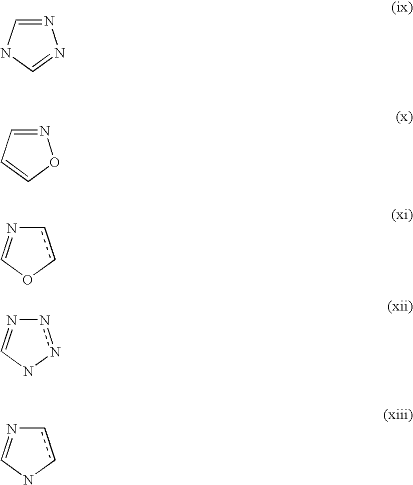
wherein when R³ is -alkyl, R⁴ is present as a hydrocarbon group, when R³ is —NO₂R⁴ is present and/or R¹ is present as a sulphamate group, and when R³ is —C(═O)R⁸, R⁴ is present and R¹ is present as a sulphamate group.

2. A compound according to claim 1 of Formula I

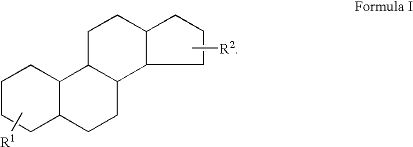

3. A compound according to claim 1 of Formula II

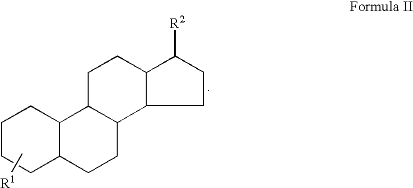

4. A compound according to claim 1 of Formula III

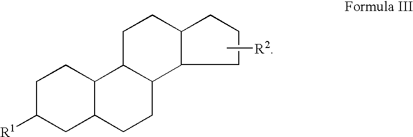

5. A compound according to claim 1 of Formula IVa or Formula IVb

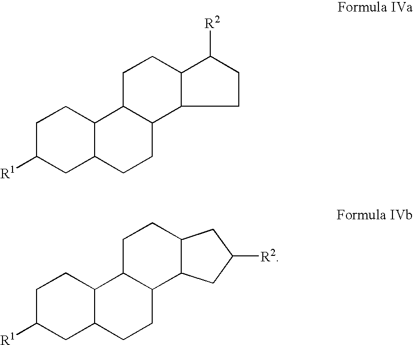

6. A compound according to claim 1 of Formula IVc

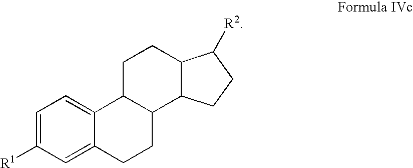

7. A compound according to claim 1 wherein R⁴ is a hydrocarbyl group or an oxyhydrocarbyl group.

8. A compound according to claim 7 wherein R⁴ is an alkoxy group.

9. A compound according to claim 8 wherein R⁴ is methoxy.

10. A compound according to claim 1 wherein R⁴ is an hydrocarbon group.

11. A compound according to claim 10 wherein R⁴ is an alkyl group.

12. A compound according to claim 11 wherein R⁴ is ethyl.

13. A compound according to claim 1 wherein R⁴ is at position 2 of the A ring.

14. A compound according to claim 1 wherein when the A ring is substituted with R¹ and R⁴, R⁴ is ortho substituted with respect to R¹.

15. A compound according to claim 1 wherein R¹ is present.

16. A compound according to claim 1 wherein R¹ is —OH or a sulphamate group.

17. A compound according to claim 1 wherein R¹ is —OH.

18. A compound according to claim 1 wherein R¹ is a sulphamate group.

19. A compound according to claim 18 wherein R¹ is a sulphamate group of the formula 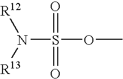
wherein R¹² and R¹³ are independently selected from H, alkyl, cycloalkyl, alkenyl and aryl, or combinations thereof, or together represent alkylene, wherein the or each alkyl or cycloalkyl or alkenyl or aryl optionally contains one or more hetero atoms or groups.

20. A compound according to claim 19 wherein at least one of R¹² and R¹³ is H.

21. A compound according to claim 20 wherein each of R¹² and R¹³ is H.

22. A compound according to claim 1 wherein L is selected from a hydrocarbyl group, —NR¹⁴— and —O—, wherein R¹⁴ is H, a hydrocarbyl group or a bond.

23. A compound according to claim 22 wherein L is selected from a hydrocarbon group, —NR¹⁴— and —O—.

24. A compound according to claim 22 wherein L is selected from an alkylene group, —NR¹⁴— and —O—.

25. A compound according to claim 22 wherein L is selected from a C₁₋₁₀ alkylene group, —NR¹⁴— and —O—.

26. A compound according to claim 22 wherein L is selected from a C₁ or C₂ alkylene group, —NR¹⁴— and —O—.

27. A compound according to claim 1 wherein groups (ix) to (xiii) are selected from optionally substituted groups of the formulae

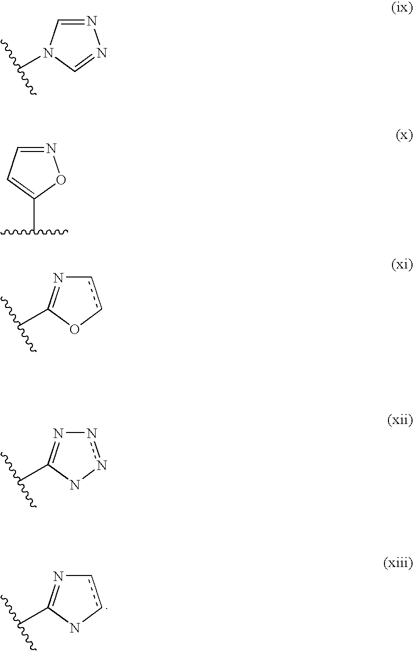

28. A compound according to claim 1 wherein R³ is —SO₂R⁵, wherein R5 is H, a hydrocarbyl group or a bond or group attached to the D ring.

29. A compound according to claim 28 wherein R⁵ is selected from H and C₁₋₁₀ alkyl.

30. A compound according to claim 28 wherein R⁵ is selected from H and C₁₋₅ alkyl.

31. A compound according to claim 28 wherein R⁵ is selected from H and C₁₋₃ alkyl.

32. A compound according to claim 28 wherein R⁵ is —CH₃.

33. A compound according to claim 28 wherein R⁵ is —O—R¹⁵-D, wherein R¹⁵ is a linker and D is a member of the D ring.

34. A compound according to claim 33 wherein R⁵ is —O—R¹⁵-D, wherein R¹⁵ is selected from —O—CH₂— and —N═CH—, and wherein D is a member of the D ring.

35. A compound according to claim 27 wherein R² is —CH₂—R³ or —NH—R³.

36. A compound according to claim 1 wherein R³ is —NO₂.

37. A compound according to claim 36 wherein R² is —CH₂—R³.

38. A compound according to claim 1 wherein R³ is —SOR⁶, wherein R⁶ is H or a hydrocarbyl group.

39. A compound according to claim 38 wherein R⁶ is selected from H and C₁₋₁₀ alkyl.

40. A compound according to claim 39 wherein R⁶ is —CH₃.

41. A compound according to claim 38 wherein R² is —CH₂—R³.

42. A compound according to claim 1 wherein R³ is —R⁷, wherein R⁷ is a halogen.

43. A compound according to claim 42 wherein R⁷ is fluorine.

44. A compound according to claim 42 wherein R² is —CH₂CH₂—R³.

45. A compound according to claim 1 wherein R³ is -alkyl.

46. A compound according to claim 45 wherein R³ is C₁₋₁₀ alkyl.

47. A compound according to claim 45 wherein R³ is C₁₋₅ alkyl.

48. A compound according to claim 45 wherein R³ is —CH₃ or —CH₂CH₃.

49. A compound according to claim 45 wherein R² is R³.

50. A compound according to claim 1 wherein R³ is —C(═O)R⁸, wherein R⁸ is H or hydrocarbyl.

51. A compound according to claim 50 wherein R⁸ is selected from H and C₁₋₁₀ alkyl.

52. A compound according to claim 50 wherein R⁸ is —CH₃.

53. A compound according to claim 50 wherein R² is —CH₂—R³.

54. A compound according to claim 1 wherein R³ is —C≡CR⁹, wherein R⁹ is H or hydrocarbyl.

55. A compound according to claim 54 wherein R⁹ is selected from H and C₁₋₁₀ alkyl.

56. A compound according to claim 54 wherein R⁹ is —CH₃.

57. A compound according to claim 54 wherein R² is —CH₂—R³.

58. A compound according to claim 1 wherein R³ is —OC(═O)NR¹⁰R¹¹, wherein R¹⁰ and R¹¹ are independently selected from H and hydrocarbyl.

59. A compound according to claim 58 wherein R¹⁰ and R¹¹ are independently selected from H and C₁₋₁₀ alkyl.

60. A compound according to claim 58 wherein R¹⁰ and R¹¹ are both H.

61. A compound according to claim 58 wherein R² is R³.

62. A compound according to claim 1 wherein R³ is

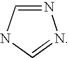

63. A compound according to claim 62 wherein R³ is

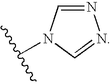

64. A compound according to claim 62 wherein R² is selected from —CH₂CH₂—R³, ═N—R³ and —NH—R³.

65. A compound according to claim 1 wherein R3 is

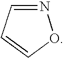

66. A compound according to claim 65 wherein R3 is

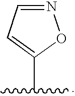

67. A compound according to claim 65 wherein R3 is

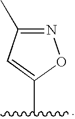

68. A compound according to claim 65 wherein R³ is selected from ═CH—R³ and —CH₂CH₂—R³.

69. A compound according to claim 1 wherein R³ is

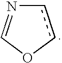

70. A compound according to claim 68 wherein R³ is

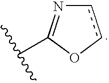

71. A compound according to claim 68 wherein R² is selected from ═CH—R³ and —CH₂CH₂—R³.

72. A compound according to claim 1 wherein R³ is

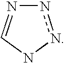

73. A compound according to claim 72 wherein R³ is

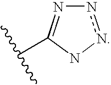

74. A compound according to claim 73 wherein R³ is selected from

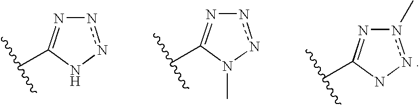

75. A compound according to claim 72 wherein R² is selected from ═CH—R³ and —CH₂CH₂—R³.

76. A compound according to claim 1 wherein R³ is

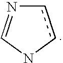

77. A compound according to claim 76 wherein R³ is

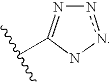

78. A compound according to claim 76 wherein R² is selected from ═CH—R³ and —CH₂CH₂—R.

79. A compound according to claim 1 wherein group R² is in an α configuration.

80. A compound according to claim 1 wherein group R² is in an α configuration on the 17 position of the D ring.

81. A compound according to claim 1 wherein group R² is in an α configuration on the 16 position of the D ring.

82. A compound according to claim 1 wherein R¹ is a sulphamate group and the compound is suitable for use as an inhibitor of oestrone sulphatase (E.C. 3.1.6.2).

83. A compound according to claim 82 wherein if the sulphamate group on the sulphamate compound were to be replaced with a sulphate group to form a sulphate compound then the sulphate compound would be hydrolysable by a steroid sulphatase enzyme (E.C.3.1.6.2).

84. A compound according to claim 83 wherein if the sulphamate group on the sulphamate compound were to be replaced with a sulphate group to form a sulphate compound and incubated with a steroid sulphatase enzyme (E.C.3.1.6.2) at a pH 7.4 and 37° C. it would provide a K, value of less than 50 □M.

85. A compound according to claim 83 wherein if the sulphamate group on the sulphamate compound were to be replaced with a sulphate group to form a sulphate compound and incubated with a steroid sulphatase enzyme (E.C.3.1.6.2) at a pH 7.4 and 37° C. it would provide a K, value of less than 50 □M.

86. A pharmaceutical composition comprising:
(a) a compound as defined in claim 1, and
(b) a pharmaceutically acceptable carrier, diluent, excipient or adjuvant.

87. A compound as defined in claim 1 for use in medicine.

88. A method of preventing and/or inhibiting tumour growth comprising administration of a medicament comprising a compound as defined in claim 1.

89. A method of treating a condition or disease associated with one or more of steroid sulphatase (STS) activity; cell cycling; apoptosis; cell growth; glucose uptake by a tumour; tumour angiogenesis; microtubules formation; and apoptosis comprising administration of a medicament comprising a compound as defined in claim 1.

90. A method of treating a condition or disease associated with one or more of adverse steroid sulphatase (STS) activity; cell cycling; apoptosis; cell growth; glucose uptake by a tumour; tumour angiogenesis; microtubules formation; and apoptosis comprising administration of a compound as defined in claim 1.

91. A method of inhibiting steroid sulphatase (STS) activity; modulating cell cycling; modulating apoptosis; modulating cell growth; preventing and/or suppressing glucose uptake by a tumour; preventing and/or inhibiting tumour angiogenesis; disrupting microtubules; or inducing apoptosis comprising administration of a compound as defined in claim 1.

92. A method of preparing a medicament for inhibiting steroid sulphatase (STS) comprising a compound as defined in claim 1.

93. A method of preparing a medicament for modulating cell growth comprising a compound as defined in claim 1.

94. A method of treatment comprising administering to a subject in need of treatment a compound as defined in claim 1.

95. A method of treatment comprising administering to a subject in need of treatment a compound as defined in claim 1 in order to inhibit steroid sulphatase (STS) activity; modulate cell cycling; modulate apoptosis; modulator cell growth; prevent and/or suppress glucose uptake by a tumour; prevent and/or inhibit tumour angiogenesis; disrupt microtubules; and/or induce apoptosis.
